(19) United States
(12) Patent Application Publication
(10) Pub. No.: US 2007/0128186 A1
(43) Pub. Date: Jun. 7, 2007

(54) Proliferative glomerulonephritis-related gene

(75) Inventors: Kyoko Takeuchi, Machida-shi (JP); Susumu Sekine, Machida-shi (JP); Yasuhiro Kikuchi, Chiyoda-ku (JP); Kazuhiro Sakurada, Machida-shi (JP)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., Chiyoda-ku (JP)

(21) Appl. No.: 11/606,133

(22) Filed: Nov. 30, 2006

Related U.S. Application Data
(62) Division of application No. 10/240,240, filed on Jun. 4, 2003, now abandoned.

(30) Foreign Application Priority Data
Mar. 29, 2000 (JP) 2000-90137

(51) Int. Cl. A61K 39/395; C12Q 1/68; G01N 33/567; A61K 38/17; C07K 14/705; C07K 16/28

(52) U.S. Cl. 424/143.1; 435/6; 435/7.2; 435/69.1; 435/320.1; 435/325; 530/350; 530/388.22; 514/12; 536/23.5

(57) ABSTRACT

A gene which is useful in searching for a therapeutic agent which restores tissue that suffered damage in a renal disease, a polypeptide encoded by the gene and an antibody which recognizes the polypeptide, are provided. A gene whose expression level changes depending on progression and recovery of the pathology in a proliferative glomerulonephritis model animal is obtained, and a polypeptide encoded by the gene and an antibody which recognizes the polypeptide are produced. These gene, polypeptide and antibody can be used in the search for an agent for restoring kidney tissue that suffered damage and an agent for diagnosing kidney disorder.

This application is a division of application Ser. No. 10/240,240 filed Jun. 4, 2003, which in turn is the national stage of PCT application No. PCT/JP01/02623 filed Mar. 29, 2001.

### TECHNICAL FIELD

The present invention relates to complementary DNA (cDNA) for mRNA obtained by using a subtraction method and differential hybridization method based on mRNA whose expression level increases at a recovery period of proliferative glomerulonephritis, and a polypeptide encoded by the cDNA. The present invention further relates to an antibody against the polypeptide, a method of detecting the polypeptide and the DNA, and a diagnostic agent and therapeutic agent for renal disease which comprise the DNA, polypeptide, antibody or the like.

### BACKGROUND ART

The kidney has a high reserve function, and in many cases even when the remaining functions are half the normal functions, symptoms due to functional disorder are not observed. Damage of nephron composed of highly differentiated cell groups is irreversible, and degeneration of tissue structure beginning in glomerulosclerosis is accompanied by tubular disorder and stromal fibrosis and ultimately results in a serious condition of renal failure which requires kidney dialysis. It is generally thought that this process is not related to the type of the primary disease, and is roughly common among the diseases. In clinical practice, administration of steroid agents, oral absorbents, antihypertensive agents, ACE inhibitors and the like, low protein diet treatment method and the like are employed for the main purpose of lightening the burden on remaining nephron and extending the period until the introduction of dialysis. However, there are many unknown points regarding the mechanism of onset and progress of renal failure, and a method for basic remedy has not been established.

In proliferative glomerulonephritis occurring in children and some animal models, it is known that natural healing occurs without progressing towards continuous decreasing of renal functions after the glomerulus or renal tubule is damaged, however, the mechanism of this natural healing is also not clarified. Analysis at the molecular level of the pathologic progression of proliferative glomerulonephritis or of the mechanism of natural healing is considered to be important for the diagnosis of renal disease and the development of therapeutic agents. An effective means for this purpose is, for example, comprehensively obtaining and analyzing a group of genes whose expression level changes in accordance with the progress of a renal disease and the recovery therefrom. It is not practical to conduct such an analysis by using tissue of an actual renal disease patient in respect of the obtainment of tissue and the non-uniformity of symptoms among patients. It is considered that by using a suitable model animal of proliferative glomerulonephritis, obtainment of comprehensive group of genes and analysis at the molecular level can be relatively easily conducted. It is considered that, in principle, genes obtained in this manner include factors which are markers of progression or recovery of pathology, as well as factors which actively promote recovery.

Several experimental models are known as a model animal of nephritis, which are mainly rats. Among these model, as for Thy-1 nephritis model [Laboratory Investigation, 55, 680 (1986)] obtained by intravenous injection into rat of an antibody (anti-Thy-1 antibody) against Thy-1.1 antigen which is present as a membrane protein of a mesangial cell (hereinafter, this nephritis model rat is referred to as “Thy-1 nephritis rat”), a considerable amount of analysis in the progression of pathology and the pathological findings has been conducted. In the Thy-1 nephritis rat, after mesangiolysis, renal tubular damage accompanying inflammatory cell infiltration to the stromata is observed, and then proliferation of mesangial cells and production of extracellular matrix occurs. It is known that reconstitution of damaged tissue takes place thereafter, and spontaneous recovery is got in approximately days beyond 2 weeks. Therefore, the Thy-1 nephritis rat is considered to be suitable as a model of progression of symptoms of proliferative glomerulonephritis and spontaneous recovery therefrom.

There have already been several reports of analysis at the molecular level of the kidney condition of the Thy-1 nephritis rat, for example, analyses regarding genes whose expression level changes depending on the kidney condition. Examples include reports regarding genes of growth factors such as TGF-β, which is considered to be involved in proliferation of mesangial cells [J. Clin. Invest., 86 453 (1990)], heparin binding EGF-like growth factor [Experimental Nephrology, 4, 271 (1996)], PDGF [Proc. Natl. Acad. Sci. USA, 88, 6560 (1991)], and FGF [J. Clin. Invest., 90, 2362 (1992)], or regarding genes such as polypeptides relating to extracellular matrix, such as type IV collagen [Kidney International, 86, 453 (1990)], laminin [Kidney International, 86, 453 (1990)], tenascin [Experimental Nephrology, 5, 423 (1997)], profilin, and CD44 [J. American Society of Nephrology, 7, 1006 (1996)].

Since growth factors such as TGF-β and PDGF in glomerulonephritis show high level of expression even in human glomerulonephritis such as lupus nephritis or IgA nephropathy as in experimental animals, these factors are considered to work as mediators of kidney failure progression through proliferation response of mesangial cells and the like, extracellular matrix production stimulation and the like [Pediatric Nephrology, 9, 495 (1995)]. It is reported that administration of PDGF neutralizing antibody [J. Exp. Med., 175, 1413 (1992)] or an inhibitor of TGF-β, decholin [Nat. Med., 2, 418 (1996)] is effective in Thy-1 nephritis rat, but, the effect of these factors has not been verified at the clinical level.

Development of a kidney protective agent by using factors involved in kidney development has been studied by Creative BioMolecules Inc. (U.S.A.), and OP-1 (BMP7) polypeptide has been provided. This is a factor belonging to BMP subfamily within TGF-β superfamily, which factor was first discovered as a factor which induces ectopic ossification [EMBO J., 9, 2085 (1990)]. This factor is expressed in mesenchymal cells surrounding the ureteric bud at a time of nephrogenesis in the fetal period, and is an important factor for interaction between the epithelium and mesenchyme. Further, it has recently been reported that this factor enables proliferation and differentiation of nephrogenic tissue by suppressing apoptosis in mesenchymal cells [Genes & Dev., 13, 1601 (1999)].

Results of a test in which the recombinant OP-1 protein of Creative BioMolecules Inc. was administered to model animals of chronic renal failure was reported at the annual convention of the American Society of Nephrology [Nikkei Biotech, Nov. 10, 1999].

In that preclinical test which was conducted by the Washington University School of Medicine (U.S.), chronic renal failure model rats in which unilateral ureter occlusion was induced were used. This model shows progressive fibrillation and renal damage which are very similar to the cicatrisation as observed in chronic renal failure patients. The results of the test indicate that OP-1 suppresses the formation of cicatricial tissue in the kidney and decreases the tubular damage. Further, in the group administered with OP-1, approximately 30% of the filtration function of a normal kidney and 65% of the blood flow amount of a kidney at normal times were maintained, showing that OP-1 also has a kidney function protection effect. In the groups administered with placebo or ACE inhibitor, filtration function and blood flow could not be determined. Tubulointerstitial lesion is more closely correlated with a decrease in kidney function than glomerular lesion, and is also a representative tissue lesion from which prognosis of a disease is predictable. Preservation of renal tubular structure was observed in only the OP-1-administered group. While the mechanism of tissue protection in the kidney is currently still not clear, the foregoing results suggest that cell groups that are the target of OP-1 are present even in the adult kidney, as in the nascent mesenchymal cells. However, various sites of action other than the kidney also exist for OP-1, and in particular, chondrogenic activity is thought to be one of the serious side effects of OP-1, and thus its clinical application is difficult to achieve. Accordingly, there is a need to find a factor, which is expressed specifically in the kidney, and capable of inducing OP-1 or controling the regenerative function of the kidney downstream of the OP-1.

Autotaxin is known as a molecule related to cell differentiation and migration, which is induced in mesenchymal cells by factors belonging to BMP or TGF-β families such as OP-1. Autotaxin has been separated and cloned from culture supernatant of human melanoma cell strain as a cytokine having cancer cell migration activity [J. Biol. Chem., 267, 683 (1996)].

Thereafter, it has been showed that autotaxin is, in the nascent period, expressed in mesenchymal cells which are being under differentiation to bone cells and cartilage cells [Mechanisms of Dev., 84, 121 (1999)], and also that the expression is increased by stimulating the precursor cells of the bone and cartilage with BMP2 [Dev. Dynam., 213, 398 (1998)]. Autotaxin is classified as PC-1 family because of the structural homology. PC-1 family is composed of 3 kinds of type II membrane proteins; PC-1, PD-1α (autotaxin) and PD-1β (B10), each having activity of phosphodiesterase I; EC 3.1.4.1/nucleotide pyrophosphatase; EC 3.6.1.9 extracellularly, and also having autophosphorylation ability. Such findings suggest that PC-1 family is expressed by stimulation of a factor belonging to BMP family, and plays an important role in cell migration, differentiation or intercellular interaction.

### DISCLOSURE OF THE INVENTION

The object of the present invention is to obtain a gene (proliferative glomerulonephritis-related gene) whose expression level changes depending on the pathologic recovery in proliferative glomerulonephritis animals, and to provide a polypeptide which is useful in searching for a therapeutic agent which restores tissue that suffered damage in a renal disease, DNA encoding the polypeptide, and an antibody which recognizes the polypeptide, as well as a method for using the same.

(A means for Solving the Object)

As a result of thorough studies to address the above-mentioned problems, the present inventors have completed the present invention.

Specifically, the present invention provides the following items (1) to (50).

(1) DNA encoding a polypeptide having an amino acid sequence selected from the group consisting of the amino acid sequences shown by SEQ ID NOS: 2, 4 and 6.

(2) DNA having a nucleotide sequence selected from the group consisting of the nucleotide sequences shown by SEQ ID NOS: 1, 3 and 5.

(3) DNA which hybridizes under stringent conditions with DNA having a nucleotide sequence selected from the group consisting of the nucleotide sequences shown by SEQ ID NOS: 1, 3 and 5, and is capable of detecting a gene whose expression level increases in tissue affected by onset of proliferative glomerulonephritis

(4) DNA having a sequence which is the same as consecutive 5 to 60 nucleotides within a nucleotide sequence selected from the group consisting of the nucleotide sequences shown by SEQ ID NOS: 1, 3 and 5.

(5) DNA having a sequence complementary with the DNA having a nucleotide sequence selected from the group consisting of the nucleotide sequences shown by SEQ ID NOs: 1, 3 and 5.

(6) A method of detecting mRNA of a gene whose expression level increases in tissue affected by onset of proliferative glomerulonephritis by using the DNA according to any one of (1) to (5) above

(7) A diagnostic agent for renal disease, which comprises the DNA according to any one of (1) to (5) above.

(8) A method of detecting a causative gene of renal disease by using the DNA according to any one of (1) to (5) above.

(9) A method of screening a substance which suppresses or promotes transcription or translation of a gene whose expression level increases in tissue affected by onset of proliferative glomerulonephritis, by using the DNA according to any one of (1) to (5) above.

(10) A method of screening a therapeutic agent for renal disease by using the DNA according to any one of (1) to (5) above.

(11) A therapeutic agent for renal disease, which comprises the DNA according to any one of (1) to (5) above.

(12) A recombinant vector which comprises the DNA according to any one of (1) to (5) above.

(13) A recombinant vector which comprises RNA of a sequence which is homologous to a sense strand of the DNA according to any one of (1) to (5) above.

(14) The vector according to (12) or (13) above, wherein the recombinant vector is a virus vector.

(15) A therapeutic agent for renal disease, which comprises the recombinant vectors according to any one of (12) to (14) above.

(16) A method of detecting mRNA of a gene whose expression level increases in tissue affected by onset of proliferative glomerulonephritis by using DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(17) A diagnostic agent for renal disease, which comprises DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(18) A method of detecting a causative gene of renal disease by using DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(19) A method of screening a substance which suppresses or promotes transcription or translation of a gene whose expression level increases in tissue affected by onset of proliferative glomerulonephritis, by using DNA having a nucleotide sequence selected from the group consisting of the nucleotide sequences shown by SEQ ID NOs: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(20) A method of screening a therapeutic agent for renal disease by using DNA having a nucleotide sequence selected from the group consisting of the nucleotide sequences shown by SEQ ID NO: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(21) A therapeutic agent for renal disease, which comprises DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(22) A recombinant vector which comprises DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(23) A recombinant vector which comprises RNA having a sequence which is homologous to a sense strand of DNA having a nucleotide sequence selected from the group consisting of the nucleotide sequences shown by SEQ ID NOS: 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 and 159.

(24) The vector according to (22) or (23) above, wherein the recombinant vector is a virus vector.

(25) A therapeutic agent for renal disease which comprises the recombinant vector according to any one of (22) to (24) above.

(26) A polypeptide encoded by the DNA according to (1) or (2) above.

(27) A polypeptide having an amino acid sequence selected from the group consisting of the amino acid sequences shown by SEQ ID NOS: 2, 4, and 6.

(28) A polypeptide having an amino acid sequence wherein one or more amino acids are deleted, substituted or added in the amino acid sequence of the polypeptide according to (26) or (27) above, and having activity involved in restoration of a kidney which suffered damage.

(29) A recombinant DNA obtained by incorporating DNA encoding the polypeptides according to any of (26) to (28) above into a vector.

(30) A transformant which is obtained by introducing the recombinant DNA according to (29) above into a host cell.

(31) A method of preparing a polypeptide wherein the transformant according to (30) above is cultured in a medium, the polypeptide according to any one of (26) to (28) above is produced and accumulated in the culture, and the polypeptide is collected from the culture.

(32) A method of screening a therapeutic agent for renal disease by using the culture which is obtained by culturing the transformant according to (30) above in a medium.

(33) A method of screening a therapeutic agent for renal disease by using the polypeptide according to any one of (26) to (28) above.

(34) A therapeutic agent for renal disease, which comprises the polypeptide according to any one of (26) to (28) above.

(35) An antibody which recognizes the polypeptide according to any one of (26) to (28) above.

(36) A method of immunologically detecting the polypeptides according to any one of (26) to (28) above by using the antibody according to (35) above.

(37) A method of screening a therapeutic agent for renal disease by using the antibody according to (35) above.

(38) A method of screening a substance which suppresses or promotes transcription or translation of a gene whose expression level increases in tissue affected by onset of proliferative glomerulonephritis by using the antibody according to (35) above.

(39) A diagnostic agent for renal disease, which comprises the antibody according to (35) above.

(40) A therapeutic agent for renal disease, which comprises the antibody according to (35) above.

(41) A method of drug delivery wherein a fusion antibody which is obtained by binding the antibody according to (35) above and an agent selected from a radioisotope, a polypeptide and a low molecular weight compound is led to a site of kidney damage.

(42) A recombinant DNA which is obtained by incorporating into a vector, DNA encoding a polypeptide having an amino acid sequence selected from the group consisting of the amino acid sequences shown by SEQ ID NOS: 8, 10, 12, 14, 16, 158 and 160.

(43) A method of screening a therapeutic agent for renal disease by using a culture which is obtained by culturing in a medium a transformant obtained by introducing the recombinant DNA according to (42) above into a host cell.

(44) A method of screening a therapeutic agent for renal disease by using a polypeptide having an amino acid sequence selected from the group consisting of the amino acid sequences shown by SEQ ID NOS: 8, 10, 12, 14, 16, 158 and 160.

(45) A therapeutic agent for renal disease, which comprises a polypeptide having an amino acid sequence selected from the group consisting of the amino acid sequences shown by SEQ ID NOS: 8, 10, 12, 14, 16, 158 and 160.

(46) A method of screening a therapeutic agent for renal disease by using an antibody which recognizes a polypeptide having an amino acid sequence selected from the group consisting of the amino acid sequences shown by SEQ ID NOS: 8, 10, 12, 14, 16, 158 and 160.

(47) A method for screening a substance which suppresses or promotes transcription or translation of a gene whose expression level increases in tissue affected by onset of proliferative glomerulonephritis, by using an antibody which recognizes a polypeptide having an amino acid sequence selected from the group consisting of the amino acid sequences shown by SEQ ID NOS: 8, 10, 12, 14, 16, 158 and 160.

(48) A diagnostic agent for renal disease, which comprises an antibody which recognizes a polypeptide having an amino acid sequence selected from the group consisting of the amino acid sequences shown by SEQ ID NOS: 8, 10, 12, 14, 16, 158 and 160.

(49) A therapeutic agent for renal disease, which comprises an antibody which recognizes a polypeptide having an amino acid sequence selected from the group consisting of the amino acid sequences shown by SEQ ID NOS: 8, 10, 12, 14, 16, 158 and 160.

(50) A method of drug delivery wherein a fusion antibody obtained by binding the antibody which recognizes a polypeptide having an amino acid sequence selected from the group consisting of the amino acid sequences shown by SEQ ID NOS: 8, 10, 12, 14, 16, 158 and 160 and an agent selected from a radioisotope, a polypeptide or a low molecular weight compound, is led to a site of kidney damage.

The DNA of the present invention is DNA of a gene whose expression level increases in tissue affected by onset of proliferative glomerulonephritis. Examples thereof include DNA encoding a polypeptide having an amino acid sequence selected from the group consisting of the amino acid sequences shown by SEQ ID NO: 2, 4 and 6, DNA having a nucleotide sequence selected from the group consisting of the nucleotide sequences shown by SEQ ID NO: 1, 3 and 5, and DNA which can hybridize under stringent conditions with said DNA and can detect a gene whose expression level changes in tissue affected by onset of proliferative glomerulonephritis.

The above-described DNA which hybridizes under stringent conditions with a nucleotide sequence selected from the group consisting of the nucleotide sequences shown by SEQ ID NO: 1, 3 and 5 means DNA obtained by using a colony hybridization method, a plaque hybridization method, a southern blotting hybridization method or the like where DNA having the nucleotide sequence shown by SEQ ID NO: 1, 3 or 5 is employed as a probe. Specifically, it means DNA which can be identified by using a filter on which colony-derived or plaque-derived DNA is immobilized, performing hybridization at 65° C. in the presence of 0.7 to 1.0 mol/l of sodium chloride and then washing the filter under a condition of 65° C. using 0.1-2-fold SSC solution (1-fold SSC solution comprises 150 mmol/l of sodium chloride and 15 mmol/l of sodium citrate). Hybridization can be performed in accordance with methods described in Molecular Cloning, A Laboratory Manual, Second Edition, Cold Spring Harbor Laboratory Press (1989) (hereinafter abbreviated to “Molecular Cloning Second Edition”); Current Protocols in Molecular Biology, John Wiley & Sons (1987-1997) (hereinafter abbreviated to “Current Protocols in Molecular Biology”); DNA Cloning 1: Core Techniques, A Practical Approach, Second Edition, Oxford University (1995); and the like. Examples of DNA which can hybridize include DNA having at least 60% homology with a nucleotide sequence shown by SEQ ID NO: 1, 3 or 5, preferably DNA having at least 80% homology, and more preferably DNA having at least 95% homology, when calculated using BLAST (J. Mol. Biol., 215, 403 (1990)), FASTA (Methods in Enzymology, 183, 63-98 (1990) and the like.

Further, DNA of the present invention also includes an oligonucleotide having partial sequence of the above-described DNA of the present invention, and an anti-sense oligonucleotide. Examples of the oligonucleotide include an oligonucleotide having the same sequence, for example as consecutive 5 to 60 nucleotides, preferably consecutive 10 to 40 nucleotides, within the nucleotide sequence selected from the nucleotide sequences shown by SEQ ID NOS: 1, 3 and 5. Examples of an anti-sense oligonucleotide include an anti-sense oligonucleotide of the above oligonucleotide.

Examples of the polypeptide of the present invention include a polypeptide (renal restoration factor) having an activity related with restoration of a kidney which suffered damage (renal restoration activity). Specific examples thereof include a polypeptide having an amino acid sequence selected from the amino acid sequences shown by SEQ ID NOS: 2, 4 and 6, or a polypeptide having an amino acid sequence derived from the amino acid sequence of the aforementioned polypeptide by deletion, substitution, or addition of one or more amino acids, and having renal restoration activity.

A protein having an amino acid sequence wherein one or more amino acids are deleted, substituted or added in the amino acid sequence shown by SEQ ID NO: 2, 4 or 6, and having activity as a growth factor of the protein, can be obtained, for example, by introducing site-directed mutation into DNA encoding a polypeptide having an amino acid sequence shown by SEQ ID NO: 2, 4 or 6 using a method of site-directed mutagenesis as described in Molecular Cloning Second Edition, Current Protocols in Molecular Biology, Nucleic Acids Research, 10, 6487 (1982), Proc. Natl. Acad. Sci. USA, 79, 6409 (1982), Gene, 34, 315 (1985), Nucleic Acids Research, 13, 4431 (1985), Proc. Natl. Acad. Sci. USA, 82, 488 (1985), or the like.

The number of the amino acids which are deleted, substituted or added is not particularly limited, but it may be the number which is deleted, substituted or added according to a known method such as site-directed mutagenesis as described above, and may be from 1 to several dozen amino acids, preferably 1 to 20, more preferably 1 to 10, and further preferably 1 to 5.

Further, in order for the polypeptide of the present invention to have an activity related with restoration of a kidney which suffered damage, the polypeptide preferably has at least 60% or more, normally 80% or more, and particularly preferably 95% or more of homology with an amino acid sequence shown by SEQ ID NO: 2, 4 or 6, when calculated by using BLAST or FASTA or the like.

The present invention is described in detail below.

1. Preparation of Proliferative Glomerulonephritis-Related Gene

(1) Production of Thy-1 Nephritis Rat

Thy-1 nephritis rat which is a model of mesangial proliferative glomerulonephritis is produced as follows in accordance with literature [Laboratory Investigation, 55, 680 (1986)]. By intravenously injecting an anti-Thy-1.1 antibody which is present as a membrane protein of mesangial cell of rat, into experimental rats such as Wistar rats at a dose of 1 mg/kg, mesangilysis occurs, and hyperplasia of mesangial stromata and mesangial cell proliferation are induced, thus enabling production of Thy-1 nephritis rat. Mesangiolysis can be detected by means of urinary protein and albumin.

(2) Preparation of Thy-1 Nephritis Rat Kidney Subtracted cDNA Library and Selection of cDNA from Library by Differential Hybridization

As proliferative glomerulonephritis-related DNA, cDNA of a gene whose expression level increases in Thy-1 nephritis rat kidney as compared with normal rat kidney is prepared as follows. First, a Thy-1 nephritis rat kidney cDNA library which was subjected to subtraction using normal rat kidney mRNA is prepared, and thereby a glomerulonephritis-related cDNA clone is concentrated. This cDNA can be obtained by performing again differential hybridization using Thy-1 nephritis rat kidney RNA and normal rat kidney RNA as probes respectively with regard to the cDNA clone in the obtained subtracted cDNA library, and selecting cDNA clone whose expression level increases in Thy-1 nephritis rat kidney.

(2)-1 Preparation of Thy-1 Nephritis Rat Kidney Subtracted cDNA Library

“Subtraction” means a method of selecting cDNA of a gene whose expression level is increased as compared with a control rat by preparing single strand cDNA from mRNA extracted from tissue or cells of a certain condition and hybridizing it with mRNA of cells of a control rat, and removing only the cDNA which hybridized to the mRNA.

There are several methods of preparing a subtracted cDNA library. The method used in the present invention is one in which subtraction is performed after preparing a Thy-1 nephritis rat kidney cDNA library by a conventional method and converting it into single strand DNA using helper phage [Proc. Natl. Acad. Sci. USA, 88, 825 (1991)]. Subtraction is performed by a method where the cDNA is hybridized with biotinylated mRNA of normal rat kidney, and streptavidin is further bonded to the hybridized biotinylated mRNA-cDNA complex, and then separation is carried out by phenol extraction.

(2)-1-A Preparation of Thy-1 Nephritis Rat Kidney cDNA Library

It is considered that the nephritic symptoms of Thy-1 nephritis rat differ depending on the number of days after intravenous injection, and that the expressing genes differ depending on the phases from deterioration of nephritis symptoms to natural healing. Therefore, kidney is extirpated from rats on each of days 2, 4, 6, 8, and 10 after injection, and RNA is individually extracted from each kidney. Extraction of RNA can be performed by the guanidine thiocyanate-cesium trifluoroacetate method [Methods in Enzymol., 154, 3 (1987)] or the acid guanidine thiocyanate/phenol/chloroform method [Analytical Biochemistry, 162, 156 (1987)] or by a method using a kit such as Fast Track mRNA Isolation Kit (Invitrogen). Since polyA is generally added to the 3′ end of mRNA, mRNA can be purified from RNA by a method using oligo(dT) Sepharose (Molecular Cloning Second Edition).

Preparation of a cDNA library from mRNA can be preformed with reference to the method described in the manual of the ZAP-c DNA preparation kit manufactured by Stratagene by preparing double strand cDNA using oligo(dT) primer and reverse transcriptase and inserting it into a cloning vector.

A cloning vector should have properties for a general cloning vector, i.e., it should be able to be replicated at a high copy number in Escherichia coli, and have a marker gene for transformation such as ampicillin resistance gene or kanamycin resistance gene, and have a multicloning site capable of cDNA insertion, and also should be such that the conversion into single strand DNA can be easily performed. Accordingly, as a cloning vector, there is used a phagemid vector which contains a replication signal IG (intergenic space) of M13 phage, and can be converted into a single strand DNA phage by means of infection with a helper phage, such as pBluescriptSK(−), pBluescriptII KS(+), pBS(−), pBC(+) [all of the foregoing are manufactured by Stratagene] or pUC118 [manufactured by Takara Shuzo]. Alternatively, there is used λ phage vector which can be converted into phagemid by in vivo excision utilizing a helper phage, such as λ ZAPII or ZAP Express (both manufactured by Stratagene). In vivo excision, the method for conversion into a single strand DNA phage and the method for purifying single strand DNA from phages in culture supernatant, can be conducted according to the manuals attached with the respective commercially available vectors.

As Escherichia coli to which a vector incorporating cDNA is introduced, any Escherichia coli which can express the introduced gene can be used. Examples thereof include Escherichia coli XL1-Blue MRF′ [manufactured by Stratagene, Strategies, 5, 81 (1992)], Escherichia coli C600 [Genetics, 39, 440 (1954)], Escherichia coli Y1088 [Science, 222, 778 (1983)], Escherichia coli Y1090 [Science, 222, 778 (1983)], Escherichia coli NM522 [J. Mol. Biol., 166, 1 (1983)], Escherichia coli K802 [J. Mol. Biol., 16, 118 (1966)], and Escherichia coli JM105 [Gene, 38, 275 (1985)].

Hybridization of cDNA with mRNA of a normal rat is used in subtraction. When single strand DNA is made from a phagemid, which of the two strands can be made is determined by the type of phagemid. Therefore, in preparing cDNA library, preparation of cDNA and the insertion direction into the vector are arranged in such a way that an anti-sense strand (strand having a complementary nucleotide sequence to the actual mRNA) can be made as single strand DNA from any cDNA clone. For example, as described in the manual attached with Stratagene's ZAPcDNA synthesis kit, cDNA synthesis can be performed by reverse transcriptase using an oligo(dT) primer having an Xho I site at the 5′ end and dNTP, as a substrate, which contains 5-methyl dCTP (which makes Xho I cleavage within cDNA after synthesis impossible) instead of dCTP. EcoR I adaptor is added to both ends of the synthesized cDNA, and the cDNA is cleaved at Xho I and inserted into EcoR I/Xho I site of vector λ ZAPII. Thereby, the EcoR I site side is always the 5′ side of the cDNA and the Xho I site side is always the 3′ side of the cDNA, and thus the insertion direction to the vector is constant. After this cDNA library is converted into a cDNA library using phagemid vector pBluescript SK(−) as a vector by in vivo excision, helper phage is infected to produce single strand DNA in which the cDNA part is an anti-sense strand.

(2)-1-B Subtraction Using Control Group Rat Kidney mRNA

By infecting the cDNA library of the phagemid vector prepared in (2)-1-A with a helper phage, single strand DNA phage is released in the culture, and cDNA which has become single strand DNA is purified and collected from the culture medium. In the case of λ phage vector, the same procedure is performed after the vector is converted into phagemid by in vivo excision (Molecular Cloning Second Edition).

The specific procedure for subtraction, the compositions of the agents and the reaction conditions can be selected according to a method described in Genes to Cells, 3 459 (1998). After biotinylating control group rat kidney mRNA prepared in (2)-1-A using Photoprobe biotin [manufactured by Vector Laboratories] or the like, the mRNA is hybridized with the above-described single strand Thy-1 nephritis rat kidney cDNA. After the hydrophobicity is increased by binding streptavidin to the cDNA hybridized with biotinylated mRNA by reacting the solution after hybridization with streptavidin which tightly binds with biotin, phenol is added to perform extraction procedure. cDNA which was not hybridized can be fractionated from the aqueous layer. cDNA hybridized with biotinylated mRNA is extracted into the phenol layer.

(2)-1-C Reverse Subtraction

In the subtraction operation described in (2)-1-B, not only cDNA of a gene whose expression level is high specifically in Thy-1 nephritis rat kidney, but also cDNA whose expression level is extremely low in both Thy-1 nephritis rat kidney and control rat kidney and whose number of clones is also small, as well as clones of only a vector where cDNA is not inserted, tend to be concentrated. However, such types of cDNA are not suitable for the object of the present invention. Accordingly, in order to select cDNA which present in certain level of clone number in Thy-1 nephritis rat kidney, hybridization and separation are performed on cDNA after subtraction and biotinylated mRNA of Thy-1 nephritis rat kidney in the same manner as in subtraction. In a reverse manner to normal subtraction, cDNA which hybridized with biotinylated mRNA is isolated from cDNA which did not hybridize and fractionated as a phenol layer. After heating the fractionated-hybridized biotinylated mRNA-cDNA after fractionation at 95° C., the cDNA and biotinylated mRNA are dissociated by quenching, and then extracted by adding water, to thereby isolate cDNA hybridized with mRNA in the aqueous layer.

(2)-1-D Preparation of cDNA Library after Subtraction

After the subtraction and reverse subtraction described in (2)-1-B and (2)-1-C, the cDNA is converted into double strand by using a suitable primer having a nucleotide sequence complementary to the sequence of vector region and DNA polymerase such as BcaBEST (manufactured by Takara Shuzo) or Klenow fragment, and then is introduced into Escherichia coli, and thereby cDNA library can be prepared again. As the method for introduction into Escherichia coli, electroporation having high transformation efficiency is preferred.

(2)-2 Differential Hybridization

In the subtracted cDNA library prepared in (2)-1, cDNA of a proliferative glomerulonephritis related gene whose expression level increases in Thy-1 nephritis rat kidney is concentrated. However, all the cDNA clones in the library are not necessarily proliferative glomerulonephritis-related genes. To select cDNA of proliferative glomerulonephritis-related genes from these cDNA clones, the respective mRNA levels in normal rat kidney and Thy-1 nephritis rat kidney are compared by northern hybridization (Molecular Cloning Second Edition) in which each cDNA clone is used as a probe or RT-PCR [PCR Protocols, Academic Press (1990)] using a primer based on a nucleotide sequence of a cDNA clone. This enables selection of cDNA of a nephritis-related gene whose expression level actually increases in Thy-1 nephritis rat kidney. Further, by performing the differential hybridization described below, it is possible to comprehensively and efficiently select cDNA clones whose expression level increases.

First, the subtracted cDNA library obtained by the method described in (2)-1 is diluted to such a concentration that individual colonies can be separated, and cultured on agar medium. The separated colonies are individually cultured under the same condition in a liquid culture medium. The culture medium is inoculated in the same amount on 2 nylon membranes, and the membranes are placed on agar medium and cultured under the same conditions to thereby grow colonies of roughly equal amounts on 2 membranes. DNA in the colonies of roughly equal amounts is thus blotted in the nylon membranes. After performing denaturation and neutralization of the DNA by a method described in Molecular Cloning Second Edition, DNA is immobilized on the membranes by ultraviolet irradiation. In the above procedure, it is preferable to separate and culture the individual separated colonies on agar medium in 96-well plates, and inoculate them onto the nylon membranes by using an automatic microdispenser suitable for a 96-well plate such as Hydra96 (manufactured by Robbins Scientific), since two membranes in which equal amounts of DNA derived from many colonies are blotted can be prepared rapidly, and the colonies can be identified easily by comparing the membrane with the original plate. Colony hybridization is performed using the entire mRNA of the Thy-1 nephritis rat kidney as a probe for one of the above membranes, and using the entire mRNA of the normal rat kidney as a probe for the other membrane. Then, by comparing the hybridization signal strengths, a clone whose expression level increases in Thy-1 nephritis rat kidney is selected.

As a probe, while it is possible to use labeled cDNA prepared by using a random primer and reverse transcriptase for the entire mRNA in the same manner as for a normal DNA probe, an RNA probe is desirable since it hybridizes more tightly to DNA on the membrane than DNA probe and imparts a strong signal. For example, by performing cDNA synthesis-reaction by the same method as described in (2)-1-A by using reverse transcriptase and an oligo(dT) primer having at its 5′ end an RNA polymerase-specific promoter sequence such as T7, T3 or SP6, cDNA having the promoter sequence at its terminus is synthesized from mRNA. By allowing RNA polymerase specific to the promoter sequence to act on this cDNA using a labeling nucleotide as a substrate, RNA probe which is uniform and contains labeled RNA at high ratio can be easily synthesized in a large amount. As a label of the probe, there can be used a radioisotope such as ³²P or ³⁵S, or a nonradioactive substance which can be easily detected, such as digoxigenin (DIG) or biotin.

After hybridizing the respective RNA probes of Thy-1 nephritis rat kidney and control rat kidney with the membranes prepared as described above, probes hybridized with each DNA colony are detected. In the detection of hybridized probes, methods suitable for the respective labeling substances can be used. Methods which can be used for detecting sensitively or quantitatively include, for example, in the case of a radioisotope, a method using autoradiography where X-ray film or an imaging plate is directly exposed; or in the case of DIG, a method where an anti-DIG antibody labeled with alkaline phosphatase is bound in accordance with instructions in the DIG system users guide (manufactured by Roche), a substrate such as CSPD which emits light by reacting with alkaline phosphatase is reacted, and then X-ray film is exposed.

If a gene which expresses at high level in kidney of a Thy-1 nephritis rat as compared with kidney of a control rat is present, the number of mRNA molecule of the gene present in the probe is also large. Therefore, even though equal amounts of DNA are blotted on a membrane, more probes will bind to a spot of cDNA corresponding to that gene. Therefore, by comparing the strengths of hybridization signals on 2 membranes on which DNA of the same cDNA clone is blotted, cDNA of a gene whose expression level increases in Thy-1 nephritis rat kidney as compared with normal rat kidney can be selected.

(3) Analysis of Nucleotide Sequence of DNA

With regard to cDNA of a gene whose expression level increases in Thy-1 nephritis rat kidney as compared with normal rat kidney obtained in the manner described above, its nucleotide sequence can be determined by using a dideoxy method [Sanger et al., Proc. Natl. Acad. Sci., USA, 74, 5463 (1977)] or a DNA sequencer.

Examples of cDNA obtained in this manner include DNA having the nucleotide sequence shown by SEQ ID NO: 1, 3, 5, 7, 9, 13, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, or 157.

By translating the obtained nucleotide sequence into an amino acid sequence, the amino acid sequence of a polypeptide encoded by the gene can be obtained. Further, by comparing the obtained nucleotide sequence with nucleotide sequences in nucleotide sequence databases such as GenBank or EMBL using a homology analysis program such as BLAST or FASTA, it is possible to confirm whether or not the obtained nucleotide sequence is a novel nucleotide sequence, and to search for a nucleotide sequence having homology with the obtained nucleotide sequence. In addition, by comparing the amino acid sequence obtained from the nucleotide sequence with the amino acid sequence databases such as SwissProt, PIR, or GenPept, it is possible to search for a polypeptide having homology with a polypeptide encoded by the nucleotide sequence, for example, a polypeptide derived from a corresponding gene in an organsms other than rat, or a family polypeptide which is estimated to have similar activity or functions.

(4) Preparation of Full-Length cDNA

The cDNA obtained in (2) may include incomplete cDNA which does not encode the full length of a polypeptide, because a part of mRNA is degraded or synthesis by reverse transcriptase is stopped on the way from the 3′ end of mRNA to the 5′ end. In analysis of a nucleotide sequence of such incomplete cDNA, all the amino acid sequence of a polypeptide encoded by the cDNA cannot be clarified. In analysis of a nucleotide sequence, it is sometimes anticipated from results of comparison with a nucleotide sequence or amino acid sequence having homology or of comparison of a length of mRNA obtained by the northern blotting method described in 5. with the length of obtained cDNA that the obtained cDNA is not full length. When the obtained cDNA is incomplete cDNA, full-length cDNA can be obtained in the manner described below.

(4)-1 Re-Searching of cDNA Library

By employing the obtained nephritis-related cDNA as a probe and performing colony hybridization or plaque hybridization (Molecular Cloning Second Edition) using Thy-1 nephritis rat kidney cDNA library, a hybridizing cDNA clone is obtained. DNA is prepared from the obtained clone by a method described in Molecular Cloning Second Edition, and DNA having the longest insert fragment is selected by cleaving with restriction enzyme. For the Thy-1 nephritis rat kidney cDNA library, a subtracted cDNA library may be prepared again. However, depending on the subtractive operation, there is a tendency for a clone containing a longer cDNA to be lost. Therefore, there is higher possibility of obtaining a full-length cDNA clone when using a pre-differentiation Thy-1 nephritis rat kidney cDNA library.

(4)-2 Raid Amplification of cDNA Ends (RACE)

By adding an adaptor oligonucleotide to both ends of Thy-1 nephritis rat kidney cDNA and performing 5′-RACE and 3′-RACE [Proc. Natl. Acad. Sci. USA, 85, 8998 (1988)] where PCR is performed with a primer based on the nucleotide sequence of the adaptor and the nucleotide sequence of the obtained cDNA clone, it is possible to obtain cDNA fragment which corresponds to external regions of the 5′-end and 3′-end of cDNA obtained in (2). The nucleotide sequence of the obtained cDNA is determined in the manner described in (3). By ligating the cDNA obtained by this method and the cDNA obtained in (2), cDNA of full length can be obtained.

Examples of full-length cDNA of a rat proliferative glomerulonephritis-related gene obtained in this manner include cDNA of a rat proliferative glomerulonephritis-related gene having the nucleotide sequence shown by SEQ ID NO: 1, 3, 5, 7, 9, 13, 17 or 157.

(4)-3 Utilization of Database Information and PCR

When performing homology analysis of a nucleotide sequence of cDNA determined in (3) with the nucleotide sequence database, in some cases, while matching with a nucleotide sequence of an known gene may not be found, matching with an EST (expressed sequence tag) which is a nucleotide sequence of a terminus region of a random cDNA clone may be found. In such case, these ESTs, ESTs having a nucleotide sequence matching with the nucleotide sequence of the ESTs, and ESTs derived from clones identical to such ESTs are collected as ESTs derived from an identical gene. By ligating the nucleotide sequences of these ESTs derived from an identical gene, a nucleotide sequence of a region that extends further to the 5′ side or 3′ side than the cDNA obtained in (2) may be sometimes found. In this case, by performing PCR employing Thy-1 nephritis rat kidney cDNA or Thy-1 nephritis rat kidney cDNA library as a template, using a forward primer having a nucleotide sequence of the 5′ end of the nucleotide sequence obtained by ligating ESTs or a reverse primer having a complementary nucleotide sequence to the nucleotide sequence of the 3′ end, there can be obtained a cDNA fragment which corresponds to external region of the 5′ end or 3′ end of a nucleotide sequence of cDNA obtained in (2). The nucleotide sequence of the obtained cDNA can be determined in the same manner as described in (3), and a cDNA of full length can be obtained by ligating with the cDNA obtained in (2). When many numbers of ESTs of rat derived from a target nephritis-related gene have been obtained from a database, it may be possible to clarify the nucleotide sequence of the full-length cDNA of a nephritis-related gene by ligating the nucleotide sequences of the collected ESTs without performing RT-PCR.

Further, after clarifying the nucleotide sequence of the obtained full-length cDNA as described above, a full-length cDNA can be obtained by preparing a primer based on the nucleotide sequence of the cDNA and performing PCR employing Thy-1 nephritis rat kidney cDNA or cDNA library as a template. Also, based on the determined nucleotide sequence of a nephritis-related gene, it is possible to chemically synthesize nephritis-related gene DNA using a DNA synthesizer. Examples of a DNA synthesizer include DNA synthesizer model 392 manufactured by Perkin-Elmer, which utilizes a phosphoramidite method.

(5) Obtaining of Human-Corresponding Gene

In order to apply a proliferative glomerulonephritis-related gene to treatment and diagnosis of proliferative glomerulonephritis in humans, a human-derived gene is necessary. In general, even if a polypeptide having the same function is of a different species, there is a high homology in the amino acid sequence, and there is also a tendency for a high homology to exist in the nucleotide sequence of the gene encoding the polypeptide. Therefore, by performing screening by hybridization under somewhat stringent conditions using cDNA library of human kidney, preferably kidney of a proliferative glomerulonephritis patient, employing rat cDNA as a probe, it is possible to obtain human cDNA. The term “somewhat stringent conditions” used herein, while differing depending on the homology of human cDNA and rat cDNA, means that southern blotting is performed under several hybridization conditions of differing degrees by employing rat cDNA as a probe for human chromosome DNA cleaved with restriction endonuclease, and the most stringent conditions of these conditions is used at which band is detected clearly. For example, in the case of a hybridization using a hybridization solution which does not contain formamide, the composition of the hybridization solution is fixed to be a salt concentration of 1 mol/l and hybridization is performed under several conditions in which the hybridization temperature is gradually changed between 68° C. and 42° C. Then, the condition is determined by washing with 2-fold SSC containing 0.5% SDS at the same temperature as hybridization. In the case of a hybridization using a hybridization solution containing formamide, the temperature (42° C.) and salt concentration (6-fold SSC) is fixed, and hybridization is performed under several conditions where the formamide concentration is gradually changed between 50% and 0%. Then, a condition is decided by washing with 6-fold SSC containing 0.5% SDS at 50° C.

Further, for a nucleotide sequence of rat cDNA obtained in (2) or (4), a search regarding the novelty and homology of the nucleotide sequence is performed in the same manner as described in (3), so as to search whether there is a nucleotide sequence of human cDNA showing high homology (specifically, 80% or more) in particular in a overall region encoding a polypeptide within the nucleotide sequence of rat cDNA. Human cDNA showing high homology is presumed to be cDNA of a human gene corresponding to the rat gene obtained in (2) or (4). Therefore, by performing RT-PCR using a primer corresponding to the nucleotide sequence of the 5′ and 3′ ends of this human DNA, and using RNA derived from human cell or tissue, preferably from kidney tissue or cell derived from kidney, and more preferably from kidney of a proliferative glomerulonephritis patient as a template, the human cDNA can be amplified and isolated. Further, while there may be cases where human cDNA found in a database is not a full-length cDNA or is merely the nucleotide sequence of an EST, in such cases also, full-length human cDNA can be obtained by the same method as described in (4) with respect to rat cDNA.

Further, analysis of a nucleotide sequence of human cDNA obtained in this manner can be performed in the same manner as described in (3), and thus the amino acid sequence of a human polypeptide encoded by that cDNA can be clarified.

Examples of human cDNA of a proliferative glomerulonephritis-related gene obtained in this manner include cDNA having the nucleotide sequences shown by SEQ ID NO: 11, 15 or 159.

In addition, for other non-human mammalians also, a corresponding gene can be obtained by using the similar method.

(6) Obtaining of Genome Gene

Genome DNA of rat- or human-gene of the present invention can be obtained by screening a genome DNA library prepared using chromosome DNA isolated from tissue or cells of a rat or human by a method such as plaque hybridization using rat or human cDNA obtained in (2) or (5) as a probe according to a method described in Molecular Cloning Second Edition. By comparing the nucleotide sequence of genome DNA and the nucleotide sequence of cDNA, it is possible to clarify the exon/intron structure of the gene. Further, in particular by employing the 5′ end region of the cDNA as a probe, the nucleotide sequence of a genome gene region which regulates transcription such as a promoter for the gene of the present invention can be clarified. This sequence is useful for analysis of the regulatory mechanism of transcription of a gene of the present invention.

Using the similar method, also for other non-human mammalians, a genome gene of the present invention can be obtained and a nucleotide sequence of a promoter region or the like can be clarified.

(7) Preparation of Oligonucleotide

Using nucleotide sequence information of DNA of the present invention obtained by the above-described method, an oligonucleotide having a partial sequence of DNA of the present invention, such as an anti-sense oligonucleotide or sense oligonucleotide, can be prepared by means of a DNA synthesizer.

Examples of the oligonucleotide include DNA having the same sequence as consecutive 5 to 60 nucleotides within a nucleotide sequence of the above DNA, or DNA having a complementary sequence with the DNA. Specific examples include DNA having the same sequence as consecutive 5 to 60 nucleotides within the nucleotide sequences shown by SEQ ID NO: 1, 3 or 5, or DNA having a complementary sequence with these DNA. In the case of using as a forward primer and reverse primer of PCR, the oligonucleotide is preferred to be an oligonucleotides of which melting temperatures (Tm) and numbers of nucleotides of both are not greatly different and which have 5 to 60 nucleotides.

Further, a derivative of these oligonucleotides (hereinafter referred to as an “oligonucleotide derivative”) can also be used as an oligonucleotide of the present invention.

Examples of the oligonucleotide derivative include an oligonucleotide derivative in which phosphodiester bond within an oligonucleotide was converted into phosphorothioate bond, an oligonucleotide derivative in which phosphodiester bond within an oligonucleotide was converted intto N3′-P5′ phosphamidate binding, an oligonucleotide derivative in which ribose and phosphodiester bond within an oligonucleotide were converted into peptide nucleic acid bond, an oligonucleotide derivative in which uracil within an oligonucleotide was substituted with C-5 propynyl uracil, an oligonucleotide derivative in which uracil within an oligonucleotide was substituted with C-5 thiazol uracil, an oligonucleotide derivative in which cytosine within an oligonucleotide was substituted with C-5 propynyl cytosine, an oligonucleotide derivative in which cytosine within an oligonucleotide was substituted with phenoxazine-modified cytosine, an oligonucleotide derivative in which ribose within an oligonucleotide was substituted with 2′-O-propylribose, and an oligonucleotide derivative in which ribose within an oligonucleotide was substituted with 2′-methoxyethoxyribose [Cell Technology, 16, 1463 (1997)].

2. Production of Proliferative Glomerulonephritis-Related Polypeptide

Hereinafter, a method for producing a proliferative glomerulonephritis-related polypeptide will be described.

Based on a full-length cDNA, a DNA fragment of an adequate length containing a region encoding the polypeptide is prepared as necessary.

By inserting the DNA fragment or full-length cDNA downstream of a promoter in an expression vector, a recombinant vector which expresses the polypeptide is constructed.

The recombinant vector is introduced into a host cell suitable for the vector.

As a host cell, any host cell which is capable of expressing the target DNA can be used as a host cell. Examples include bacteria belonging to the genus Escherichia, Serratia, Corynebacterium, Brevibacterium, Pseudomonas, Bacillus, Microbacterium or the like; yeast belonging to the genus Kluyveromyces, Saccharomyces, Shizosaccharomyces, Trichosporon, Schwanniomyces or the like; animal cells, and insect cells.

As an expression vector, any vector which is capable of autonomous replication in a host cell or integration into a chromosome in a host cell, and contains a promoter in a position that can work in transcribing proliferative glomerulonephritis-related DNA, can be used.

When bacteria is used as a host cell, a proliferative glomerulonephritis-related DNA recombinant vector is preferably a recombinant vector which is capable of autonomous replication in the bacteria and comprises a promoter, a ribosome binding sequence, proliferative glomerulonephritis-related DNA, and a transcription termination sequence. The vector may comprise a gene which controls a promoter.

Examples of an expression vector include pBTrp2, pBTac1, pBTac2 (all commercially available from Boeringer Mannheim), pKK233-2 (manufactured by Amersham Pharmacia Biotech), pSE280 (manufactured by Invitrogen), pGEMEX-1 (manufactured by Promega), pQE-8 (manufactured by QIAGEN), pKYP10 (Japanese Published Unexamined Patent Application No. 110600/83), pKYP200 [Agricultural Biological Chemistry, 48, 669 (1984)], pLSA1 [Agric. Biol. Chem., 53, 277 (1989)], pGEL1 [Proc. Natl. Acad. Sci. USA, 82, 4306 (1985)], pBluescript II SK(−) (manufactured by Stratagene), pGEX (manufactured by Amersham Pharmacia Biotech), pET-3 (manufactured by Novagen), pTerm2 (U.S. Pat. No. 4,686,191, U.S. Pat. No. 4,939,094, U.S. Pat. No. 5,160,735), pSupex, pUB110, pTP5, pC194, and pEG400 [J. Bacteriol., 172, 2392 (1990)].

As an expression vector, an expression vector, in which the distance between a Shine-Dalgamo sequence which is a ribosome binding sequence, and an initiation codon is adjusted at an adequate distance (for example, 6 to 18 nucleotides), is preferably used.

As a promoter, any promoter can be used as long as it can work in a host cell. Examples include promoters derived from Escherichia coli or phage such as trp promoter (Ptrp), lac promoter (Plac), PL promoter, PR promoter and T7 promoter; SPO1 promoter, SPO2 promoter, and penP promoter. Further, an artificially modified promoter such as a promoter having two Ptrp in tandem (Ptrp×2), tac promoter, letI promoter [Gene, 44, 29 (1986)], or lacT7 promoter can also be used.

By substituting nucleotides of a region encoding a polypeptide of proliferative glomerulonephritis-related DNA of the present invention so as to make an optimal codon for expression of the host, the production efficacy of the target polypeptide can be increased.

While a transcription termination sequence is not necessarily required for expression of proliferative glomerulonephritis-related DNA of the present invention, ideally, a transcription termination sequence is desirably placed directly downstream a structural gene.

Examples of a host cell include a microorganism belonging to the genus Escherichia, Serratia, Corynebacterium, Brevibacterium, Pseudomonas, and Bacillus, such as Escherichia coli XL1-Blue, Escherichia coli XL2-Blue, Escherichia coli DH1, Escherichia coli MC1000, Escherichia coli KY3276, Escherichia coli W1485, Escherichia coli JM109, Escherichia coli HB101, Escherichia coli No. 49, Escherichia coli W3110, Escherichia coli NY49, Bacillus subtilis, Bacillus amyloliguefaciens, Brevibacterium ammoniagenes, Brevibacterium immariophilum ATCC14068, Brevibacterium saccharolyticum ATCC14066, Corynebacterium glutamicum ATCC13032, Corynebacterium glutamicum ATCC14067, Corynebacterium glutamicum ATCC13869, Corynebacterium acetoacidophilum ATCC13870, Microbacterium ammoniaphilum ATCC15354, and Pseudomonas sp. D-0110.

As a method of introducing a recombinant vector, any method can be used as long as it is a method of introducing DNA to a host cell. Examples thereof include the method using calcium ion [Proc. Natl. Acad. Sci. USA, 69, 2110 (1972)], the protoplast method (Japanese Published Unexamined Patent Application No. 248394/88), and the method described in Gene, 17, 107 (1982) or Molecular & General Genetics, 168, 111 (1979).

When yeast is used as a host cell, examples of an expression vector include YEp13 (ATCC37115), YEp24 (ATCC37051), YCp50(ATCC37419), pHS19, and pHS15.

As a promoter, any promoter which can work in yeast may be used. Examples thereof include PHO5 promoter, PGK promoter, GAP promoter, ADH promoter, gal 1 promoter, gal 10 promoter, heat shock protein promoter, MFa1 promoter, and CUP 1 promoter.

Examples of a host cell include Saccharomyces cerevisiae, Schizosaccharomyces pombe, Kluyveromyces lactis, Trichosporon pullulans, and Schwanniomyces alluvius.

As a method of introducing a recombinant vector, any method can be used as long as it is a method of introducing DNA to yeast. Examples include the electroporation method [Methods in Enzymol., 194, 182 (1990)], the spheroplast method [Proc. Natl. Acad. Sci. USA, 75, 1929 (1978)], the lithium acetate method [J. Bacteriol., 153, 163 (1983)], and the method described in Proc. Natl. Acad. Sci. USA, 75, 1929 (1978).

When an animal cell is used as a host cell, examples of an expression vector include pcDNAI (manufactured by Invitrogen), pcDM8 (manufactured by Invitrogen), pAGE107 [Japanese Published Unexamined Patent Application No. 22979/91; Cytotechnology, 3, 133 (1990)], pAS3-3 (Japanese Published Unexamined Patent Application No. 227075/90), pCDM8 [Nature, 329, 840 (1987)], pcDNAI/Amp (manufactured by Invitrogen), pREP4 (manufactured by Invitrogen), pAGE103 [J. Biochem., 101, 1307 (1987)], and pAGE210.

As a promoter, any promoter that work in an animal cell may be used, and examples include an immediate early gene promoter of cytomegalovirus (human CMV), early promoter of SV40, promoter of retrovirus, metallothionein promoter, heat shock protein promoter, and SR α promoter. Also, an immediate early gene enhancer of human CMV may be used together with a promoter.

Examples of a host cell include Namalwa cell which is a human cell, COS cell which is a simian cell, CHO cell which is a cell of a Chinese hamster, and HBT 5637 [Japanese Published Unexamined Patent Application No. 299/88].

As a method of introducing a recombinant vector, any method which can introduce DNA to an animal cell can be used. For example, the electroporation method [Cytotechnology, 3, 133 (1990)], the calcium phosphate method (Japanese Published Unexamined Patent Application No. 227075/90), the lipofection method [Proc. Natl. Acad. Sci., USA, 84, 7413 (1987); Virology, 52, 456 (1973)] or the like can be used. Obtaining and culturing of a transformant can be carried out in accordance with the method described in Japanese Published Unexamined Patent Application No. 227075/90 or Japanese Published Unexamined Patent Application No. 257891/90.

When an insect cell is used as a host, a polypeptide can be expressed according to the method described in, for example, Baculovirus Expression Vectors, A Laboratory Manual, Oxford University Press (1994); Current Protocols in Molecular Biology; Bio/Technology 6, 47 (1988); or the like.

Specifically, after co-introducing a recombinant gene introduction vector and baculovirus into an insect cell to obtain a recombinant virus in the insect cell culture supernatant, the insect cell is infected with the recombinant virus to thereby express a polypeptide.

Examples of a vector for introduction of gene include pVL1392, pVL1393 and pBlueBacIII (all manufactured by Invitrogen).

As a baculovirus, for example, Autographa californica nuclear polyhedrosis virus which is a virus infecting insects of Barathra, or the like can be used.

As an insect cell, Sf9 and Sf21 which are ovarian cells of Spodoptera frugiperda [Baculovirus Expression Vectors, A Laboratory Manual, W. H. Freeman and Company, New York, (1992)]; High 5 which is an ovarian cell of Trichoplusia ni (manufactured by Invitrogen); or the like, can be used.

Examples of a method of co-introducing the above recombinant gene introduction vector and the above baculovirus into an insect cell to prepare a recombinant virus, include the calcium phosphate method [Japanese Published Unexamined Patent Application No. 227075/90] and the lipofection method [Proc. Natl. Acad. Sci., USA, 84, 7413 (1987)].

As a method of expressing a gene, direct expression may be used, and also secretion production, fusion polypeptide expression or the like can be performed in accordance with a method described in Molecular Cloning Second Edition or the like.

In the case of expression by means of a yeast, an animal cell or an insect cell, a polypeptide to which a sugar or sugar chain is added can be obtained.

A transformant which carries a recombinant vector incorporating proliferative glomerulonephritis-related DNA is cultured in a culture medium to produce and accumulate proliferative glomerulonephritis-related polypeptide in the culture, and then the polypeptide is collected from the culture to prepare proliferative glomerulonephritis-related polypeptide.

The method for culturing a transformant for preparing a proliferative glomerulonephritis-related polypeptide of the present invention in a culture medium can be performed in accordance with a conventional method used in culturing of a host cell.

When a transformant of the present invention employs a prokaryote such as Escherichia coli or a eukaryote such as yeast as a host cell, a medium for culturing the transformant may be either a natural medium or a synthetic medium, as long as it is a medium which contains carbon source that the host cell can assimilate, nitrogen source, minerals and the like and by which culturing of the transformant can be efficiently carried out.

As a carbon source, any carbon source which the respective host cell can assimilate may be used. For example, there can be used a carbohydrate such as glucose, fructose, sucrose, molasses containing these, starch or starch hydrolysate; organic acid such as acetic acid or propionic acid; and alcohol such as ethanol or propanol.

As a nitrogen source, there is used various inorganic acids such as ammonia, ammonium chloride, ammonium sulfate, ammonium acetate, and ammonium phosphate, or organic acid ammonium salts, other nitrogen containing compounds, as well as peptone, meat extract, yeast extract, corn steep liquor, casein hydrolysate, soybean cake and soybean cake hydrolysate, various microorganisms obtained by fermentation and their digest product, and the like.

As a mineral, there can be used potassium dihydrogenphosphate, dipotassium hydrogenphosphate, magnesium phosphate, magnesium sulfate, sodium chloride, ferrous sulfate, manganese sulfate, copper sulfate, calcium carbonate, and the like.

Culturing is performed under aerobic conditions such as shaking culture or submerged spinner culture. Culturing temperature may be from 15 to 40° C., and culturing period is normally between 16 hours to 7 days. pH during culturing is maintained between 3.0 to 9.0. Adjustment of pH is performed by using inorganic or organic acid, alkaline solution, urea, calcium carbonate, ammonia, or the like.

Further, if necessary, antibiotics such as ampicillin or tetracycline may be added to the culture medium during culturing.

When culturing a transformant obtained by using a recombinant vector having an inducible promoter as a promoter, an inducer may be added to the culture medium, if necessary. For example, when culturing a transformant that used a recombinant vector containing lac promoter, isopropyl-β-D-thiogalactopyranoside (IPTG) or the like may be added to the culture medium, and when culturing a transformant that obtained by using a recombinant vector containing trp promoter, indoleacrylic acid (IAA) or the like may be added to the culture medium.

As a medium for culturing a transformant obtained by employing an animal cell as a host cell, the generally used RPMI 1640 [The Journal of the American Medical Association, 199, 519 (1967)], Eagle's MEM medium [Science, 122, 501 (1952)], Dulbecco's modified MEM medium [Virology, 8, 396 (1959)], 199 medium [Proceeding of the Society for the Biological Medicine, 73, 1 (1950)], or a medium in which fetal calf serum or the like was added to these mediums, or the like can be used.

Culturing is normally performed for 1 to 7 days under conditions of a pH of 6 to 8, and a temperature of 30 to 40° C. in the presence of 5% CO₂.

Further, if necessary during culturing, antibiotics such as kanamycin or penicillin may be added to the medium.

As a medium for culturing a transformant obtained by employing an insect cell as a host cell, the generally used TNM-FH medium (manufactured by Pharmingen), Sf-900 II SFM medium (manufactured by Life Technologies), ExCell400 and ExCell405 (both manufactured by JRH Biosciences), Grace's Insect Medium [Grace, T. C. C., Nature, 195, 788 (1962)], or the like can be used.

Culturing is normally performed for 1 to 5 days under conditions of a pH of 6 to 7 and a temperature of 25 to 30° C.

Further, if necessary during culturing, an antibiotic such as gentamicin may be added to the medium.

To isolate and purify a proliferative glomerulonephritis-related polypeptide from the culture of a transformant, a conventional method for isolating and purifying a polypeptide may be used.

For example, when a polypeptide is produced in soluble form within cell, after completion of culturing, the cell is recovered by centrifugation and is suspended in an aqueous buffer, and then the cell is disrupted by means of, for example, an ultrasonic homogenizer, a French press, a Manton-Gaurin homogenizer, or Dyno-Mill, to obtain cell-free extract. From supernatant obtained by centrifuging the cell-free extract, purified polypeptide can be obtained using, either alone or in combination, a conventional technique for isolating and purifying a polypeptide, such as solvent extraction, salting-out using ammonium sulfate or the like, desalting, precipitation using an organic solvent, anion exchange chromatography using a resin such as diethylaminoethyl (DEAE)-Sepharose or DIAION HPA-75 (manufactured by Mitsubishi Chemical Corporation), cation exchange chromatography using a resin such as S-Sepharose FF (manufactured by Amersham Pharmacia Biotech), hydrophobic chromatography using a resin such as butyl-Sepharose or phenyl-Sepharose, gel filtration using a molecular sieve, affinity chromatography, chromatofocusing, and an electrophoresis method such as isoelectric focusing.

Further, when a polypeptide is produced as inclusion bodies within a cell, after recovering the cell, the cell is disrupted and is subjected to centrifugation, to thereby recover the inclusion bodies of the polypeptide as a precipitation fraction.

The recovered inclusion bodies of the polypeptide is solubilized with a protein denaturing agent. The structure of the polypeptide is returned to a normal steric structure by lowering the concentration of the protein denaturing agent in the lysate by subjecting the lysate to dilution or dialysis, and then purified polypeptide is obtained by the method of isolation and purification as described above.

When a polypeptide or its glycosylated derivative or the like is secreted to outside the cell, the polypeptide or its glycosylated derivative or the like can be recovered from the culture supernatant. Specifically, by recovering the culture supernatant from the culture by using a technique such as centrifugation, and then using the method of isolation and purification as described above, a purified polypeptide can be obtained from the culture supernatant.

Examples of a polypeptide obtained in this manner include a polypeptide having an amino acid sequence shown by SEQ ID NO: 2, 4, 6, 8, 10, 12, 14, 16, 158 or 160.

Further, the polypeptide of the present invention can also be produced by a chemical synthesis method such as the Fmoc method (fluorenylmethyloxycarbonyl method) or the tBoc method (t-butyloxycarbonyl method). Further, synthesis can also be conducted by using a peptide synthesizer such as a peptide synthesizer manufactured by Advanced ChemTech (USA), Perkin-Elmer, Amersham Pharmacia Biotech, Protein Technology Instrument (USA), Synthecell-Vega (USA), PerSeptive (USA) or Shimadzu Corporation.

3. Preparation of Antibody Specifically Recognizing Proliferative Glomerulonephritis-Related Polypeptide

By using, as an antigen, a purified full length or partial fragment of a proliferative glomerulonephritis-related polypeptide or a synthetic peptide having a partial amino acid sequence of polypeptide of the present invention, an antibody which recognizes a proliferative glomerulonephritis-related polypeptide, such as a polyclonal antibody or monoclonal antibody, can be produced.

(1) Production of Polyclonal Antibody

A polyclonal antibody can be produced by subcutaneously, intravenously or intraperitoneally administering an antigen to an animal with using a purified full length or partial fragment of the polypeptide of the present invention or a peptide having a partial amino acid sequence of the protein of the present invention as an antigen, together with an adequate adjuvant [for example, Complete Freund's Adjuvant, aluminum hydroxide gel, or pertussis vaccine].

As an animal to be administered, rabbit, goat, rat, mouse, hamster, or the like can be used.

A dosage of antigen is preferably 50 to 100 μg per animal.

When a peptide is used, it is preferred to use an antigen in which the peptide is covalently bonded to a carrier protein such as keyhole limpet haemocyanin or bovine thyroglobulin. A peptide to be used as an antigen can be synthesized by a peptide synthesizer.

Administration of the antigen is performed 3 to 10 times at 1- to 2-week intervals after the initial administration. On days 3 to 7 after each administration, a blood sample is collected from ocular fundus plexus venosus, and the reaction of the serum with the antigen used for immunity is confirmed by enzyme linked immunoassay [Enzyme Linked Immunoassay (ELISA): Igaku-Shoin Publication (1976), Antibodies-A Laboratory Manual, Cold Spring Harbor Laboratory (1988)].

For the antigen used for immunity, serum is obtained from a non-human mammalian whose serum shows a sufficient antibody titer, and then the serum is separated and purified to obtain a polyclonal antibody.

Examples of a separation and purification method include a method using, either alone or in combination, techniques such as centrifugation, salting-out by 40-50% saturated ammonium sulfate solution, caprylic acid precipitation [Antibodies, A Laboratory Manual, Cold Spring Harbor Laboratory, (1988)], and chromatography using DEAE-Sepharose column, anion exchange column, protein A- or G-colunm or gel filtration column, or the like.

(2) Production of Monoclonal Antibody

(a) Preparation of Antibody Producing Cell

For a partial fragment polypeptide of the polypeptide of the present invention used for immunity, a rat whose serum shows a sufficient antibody titer is provided as a source of antibody producing cells.

On day 3 to 7 after final administration of an antigen to a rat showing such antibody titer, the spleen is extirpated from the rat.

The spleen is macerated in MEM medium (manufactured by Nissui Pharmaceutical Co., Ltd.), loosen with a pin set, subject to centrifugation for 5 min at 1,200 rpm, and then the supernatant is discarded.

After treating spleen cells of the obtained precipitation fraction with tris-ammonium chloride buffer (pH 7.65) for 1-2 minutes to remove erythrocytes, the cells are washed with MEM medium 3 times, and the obtained spleen cells are used as antibody producing cells.

(b) Preparation of Myeloma Cells

An established cell line obtained from a mouse or rat is used as myeloma cells. For example, 8-azaguanine resistant mouse (BALB/c derived) myeloma cell line P3-X63Ag8-U1 (hereinafter referred to as “P3-U1”) [Curr. Topics. Microbiol. Immunol., 81, 1 (1978), Europ. J. Immunol., 6, 511 (1976)], SP2/0-Ag14(SP-2) [Nature, 276, 269 (1978)], P3-X63-Ag8653(653) [J. Immunol., 123, 1548 (1979)], P3-X63-Ag8(X63) [Nature, 256, 495 (1975)] or the like can be used. These cell lines are subcultured with 8-azaguanine medium [a medium obtained by adding glutamine (1.5 mmol/l), 2-mercaptoethanol (5×10⁻⁵ mol/l), gentamicin (10 μg/ml), and fetal calf serum (FCS) (manufactured by CSL, 10%) to RPMI-1640 medium (the obtained medium is hereinafter referred to as “normal medium”), and then adding 8-azaguanine (15 μg/ml)]. The cells are cultured in the normal medium 3-4 days prior to cell fusion. 2×10⁷ or more of the cells is used for fusion.

(c) Preparation of Hybridoma

The antibody producing cells obtained in (a) and the myeloma cells obtained in (b) are washed well in MEM medium or PBS (disodium hydrogenphosphate 1.83 g, potassium dihydrogenphosphate 0.21 g, sodium chloride 7.65 g, 1 liter of distilled water, pH 7.2), and the cells are mixed such that the number of cells is of a ratio where antibody producing cells: myeloma cells=5:1 to 10:1, and centrifuged for 5 minutes at 1,200 rpm, and the supernatant is discarded.

The cell groups in the obtained precipitation fraction are loosened well, and 0.2 to 1 ml of a solution obtained by mixing 2 g of polyethylene glycol-1000 (PEG-1000), 2 ml of MEM and 0.7 ml of dimethyl sulfoxide (DMSO) is added to the cell groups per 10⁸ antibody producing cells while stirring at a temperature of 37° C., and further 1 to 2 ml of MEM medium is added several times at 1-2 minute intervals.

After addition, MEM medium is added so as to make the total volume of 50 ml. The prepared fluid is centrifuged at 900 rpm for 5 minutes, and then the supernatant is discarded. After gently loosening the cells in the obtained precipitation fraction, the cells are gently suspended in 100 ml of HAT medium [medium obtained by adding hypoxanthine (10⁻⁴ mol/l), thymidine (1.5×10⁻⁵ mol/l) and aminopterin (4×10⁻⁷ mol/l) to normal solution] by pipetting.

The suspension is dispensed onto 96-well culture plates at 100 μl/well, and is cultured in 5% CO₂ incubator at 37° C. for 7-14 days.

After culturing, an aliquot of the culture supernatant is taken out, and a hybridoma which specifically reacts with a partial fragment polypeptide of the protein of the present invention is selected by an enzyme immunoassay method described in Antibodies [Antibodies, A Laboratory Manual, Cold Spring Harbor Laboratory, Chapter 14 (1988)] or the like.

Specific examples of an enzyme immunoassay method include the following method.

At the time of immunization, a partial fragment polypeptide of the polypeptide of the present invention used as an antigen is coated on a suitable plate, and is reacted with the hybridoma culture supernatant or purified antibody obtained in (d) below as primary antibody. Then, anti-rat or anti-mouse immunoglobulin antibody labeled with biotin, an enzyme, a chemiluminescent substance or a radioactive compound or the like is reacted as a secondary antibody, and a reaction suitable for the labeling substance is carried out. Then, those which specifically react with the protein of the present invention are selected as hybridomas which produce a monoclonal antibody of the present invention.

Using the hybridomas, cloning is repeated two times by limiting dilution method [using HT medium (medium obtained by excluding aminopterin from HAT medium) at the first time, and normal medium at the second time], and those which show a stable and strong antibody titer are selected as a hybridoma line which produces a monoclonal antibody of the present invention.

(d) Preparation of Monoclonal Antibody

5×10⁶ to 20×10⁶cells/mouse of hybridoma cells which produce a monoclonal antibody of the present invention, obtained in (c) above are intraperitoneally injected to Pristane-treated (intraperitoneally injecting 0.5 ml of 2,6,10,14-tetramethylpentadecane (Pristane), and growing animals for 2 weeks) 8-10 week-old mice or nude mice. After 10 to 21 days, the hybridoma becomes an ascites tumor.

Ascites fluid is taken out from a mouse in which an ascites tumor developed, and centrifuged at 3,000 rpm for 5 minutes to remove solid content.

A monoclonal antibody can be purified and obtained from the obtained supernatant by using the same method as that used for the polyclonal antibody.

The determination of the subclass of the antibody is carried out by using a mouse monoclonal antibody typing kit or a rat monoclonal antibody typing kit. The amount of the polypeptide is calculated by the Lowry method or by the absorbance at 280 nm.

4. Method of Preparing Recombinant Virus Vector which Produce Proliferative Glomerulonephritis-Related Polypeptide

Hereinafter, a method of preparing a recombinant virus vector for producing the proliferative glomerulonephritis-related polypeptide of the present invention in specific human tissue is described.

Based on the full length cDNA of the proliferative glomerulonephritis-related gene, a DNA fragment of a suitable length which contains a region encoding the polypeptide is prepared if necessary.

By inserting the DNA fragment or the full length cDNA downstream of a promoter within a virus vector, a recombinant virus vector is constructed.

In the case of an RNA virus vector, cRNA homologous with full length cDNA of the proliferative glomerulonephritis-related gene or an RNA fragment homologous with a DNA fragment of a suitable length which contains a region encoding the polypeptide is prepared, and inserted downstream of a promoter within a virus vector to thereby construct a recombinant virus. For the RNA fragment, in addition to double-stranded RNA, either single strand of the sense strand or antisense strand may also be selected depending on the type of virus vector. For example, in the case of a retrovirus vector, RNA homologous to a sense strand is selected, and in the case of Sendai virus, RNA homologous to an antisense strand is selected.

The recombinant virus vector is introduced into a packaging cell suitable for the vector.

As a packaging cell, any cell can be used so long as it can supplement for the deleted polypeptide of a recombinant virus vector in which at least one of the genes which encode a polypeptide necessary for packaging of a virus is deleted. For example, human kidney-derived HEK293 cell, mouse fibroblast NIH3T3 or the like can be used. Examples of a polypeptide supplemented by packaging cell include, in the case of a retrovirus vector, polypeptides such as mouse retrovirus-derived gag, pol, and env; in the case of a lentivirus vector, polypeptides such as HIV virus-derived gag, pol, env, vpr, vpu, vif, tat, rev, and nef; in the case of an adenovirus vector, polypeptides such as adenovirus-derived E1A-E1B; in the case of an adeno associated virus, polypeptides such as Rep (p5, p19, p40) and Vp (Cap); and in the case of Sendai virus, polypeptides such as NP, P/C, L, M, F, and HN.

As a virus vector, a virus vector which can produce a recombinant virus in the above-described packaging cell and contain a promoter in a position that work in transcription of proliferative glomerulonephritis-related DNA in a target cell is used. As a plasmid vector, MFG [Proc. Natl. Acad. Sci. USA, 92, 6733-6737 (1995)], pBabePuro [Nucleic Acids Res., 18, 3587-3596 (1990)], LL-CG, CL-CG, CS-CG, CLG [Journal of Virology, 72, 8150-8157 (1998)], pAdex1 [Nucleic Acids Res., 23, 3816-3821 (1995)], or the like is used. As a promoter, any promoter can be used so long as they can work in human tissue. Examples include IE (immediate early) gene promoter of cytomegalovirus (human CMV), early promoter of SV40, retrovirus promoter, metallothionein promoter, heat shock protein promoter, and SR α promoter. Further, human CMV IE gene enhancer may also be used together with the promoter.

Examples of a method for introducing a recombinant virus vector into a packaging cell include the calcium phosphate method (Japanese Published Unexamined Patent Application No. 227075/90) and the lipofection method [Proc. Natl. Acad. Sci. USA, 84, 7413 (1987)].

5. Method for Detecting mRNA of Proliferative Glomerulonephritis-Related Gene

Hereinafter, a method for detecting mRNA of a proliferative glomerulonephritis related gene by using proliferative glomerulonephritis related DNA of the present invention is described.

Examples of DNA which can be used in this method include DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159.

Examples of a method for detecting an expression level or structural change of proliferative glomerulonephritis-related gene mRNA include (1) northern blotting, (2) in situ hybridization, (3) quantitative PCR, (4) differential hybridization, (5) a DNA chip method, and (6) RNase protection assay.

As a specimen to be subjected to the analysis according to the above method, mRNA or total RNA obtained from a biological specimen such as kidney tissue, blood serum, or saliva obtained from a kidney patient or a healthy subject, or a primary cultured cell specimen obtained by collecting cells from such biological specimen and culturing them in an adequate medium in vitro (hereinafter, the mRNA and total RNA are referred to as “specimen-derived RNA”) is used. Further, tissue which was obtained from the biological specimen and isolated as paraffin section or cryostat section can also be used.

In the northern blotting method, by separating the specimen-derived RNA by gel electrophoresis, transcribing it on support such as a nylon filter, and performing hybridization using a labeled probe prepared from DNA of the present invention and washing, the expression level as well as the structural change of mRNA derived from proliferative glomerulonephritis-related gene can be detected by detecting a band specifically bonded to mRNA derived from proliferative glomerulonephritis-related gene. In performing hybridization, incubation is carried out under conditions in which a probe and the mRNA derived from the proliferative glomerulonephritis-related gene in the specimen-derived RNA can form a stable hybrid. To prevent false positive, it is preferable that hybridization and washing process are performed under highly stringent conditions. The conditions are determined according to several factors including temperature, ionic strength, nucleotide composition, probe length, and formamide concentration. These factors are described in, for example, Molecular Cloning Second Edition.

A labeled probe used in northern blotting can be prepared, for example, by incorporating a radioisotope, biotin, a fluorescent group, a chemiluminescent group or the like into DNA of the present invention or an oligonucleotide designed from the nucleotide sequence of the DNA in accordance with a known method (e.g. nick translation, random priming, or kinasing). Since the amount of binding of the labeled probe reflects the expression level of mRNA derived from proliferative glomerulonephritis-related gene, the expression level of mRNA derived from proliferative glomerulonephritis-related gene can be determined by determining the amount of bonded labeled probe. Further, by analyzing the labeled probe binding sites, structural changes of the mRNA derived from the proliferative glomerulonephritis-related gene can be known.

The expression level of mRNA derived from the proliferative glomerulonephritis-related gene can be detected according to in-situ hybridization method where hybridization and washing processes are carried out by using the above labeled probe and tissue obtained from a living organism and isolated as paraffin section or cryostat section. In the in-situ hybridization method, in order to prevent false positive, it is desirable that the hybridization and washing processes are performed under highly stringent conditions. The conditions are determined according to several factors including temperature, ionic strength, nucleotide composition, probe length, and formamide concentration. These factors are described in, for example, Current Protocols in Molecular Biology.

A method for detecting mRNA derived from proliferative glomerulonephritis-related gene by quantitative PCR, differential hybridization or a DNA chip method can be performed according to a method based on synthesizing cDNA using specimen-derived RNA, an oligo(dT) primer or random primer, and reverse transcriptase (hereinafter, the cDNA is referred to as “specimen-derived cDNA”). When the specimen-derived RNA is mRNA, either of the above-mentioned primers can be used, but when the specimen-derived RNA is total RNA, it is necessary to use oligo(dT) primer.

In quantitative PCR, by performing PCR using a primer designed based on a nucleotide sequence of proliferative glomerulonephritis-related DNA of the present invention and employing specimen-derived cDNA as a template, DNA fragments derived from mRNA derived from proliferative glomerulonephritis-related gene are amplified. Since the amount of amplified DNA fragments reflects the expression level of mRNA derived from proliferative glomerulonephritis-related gene, it is possible to determine the level of mRNA derived from proliferative glomerulonephritis-related gene by using DNA encoding actin or G3PDH (glyceraldehyde 3-phosphate dehydrogenase) or the like as an internal control. Further, by separating the amplified DNA fragments by gel electrophoresis, structural changes of the mRNA derived from proliferative glomerulonephritis-related gene can also be known. In this detection method, it is desirable to use an adequate primer that works specifically and efficiently to amplify the target sequence. An adequate primer can be designed on the basis of conditions such as not causing binding between primers or within primers, specifically binding to target cDNA at an annealing temperature, disconnecting from target cDNA under denaturing conditions, and the like. It is necessary that quantitative determination of amplified DNA fragments be performed during PCR reaction in which amplification products are increasing exponentially. Such PCR reaction can be confirmed by recovering the amplified DNA fragments produced at each reaction and performing quantitative analysis by gel electrophoresis.

Using specimen-derived cDNA as a probe, variations in an expression level of mRNA derived from proliferative glomerulonephritis-related gene can be detected by performing hybridization and washing for filter or support such as slide glass or silicon on which DNA of the present invention is immobilized. The methods based on this principle include the differential hybridization method [Trends in Genetics, 7, 314-317 (1991)] and DNA chip method [Genome Research, 6, 639-645 (1996)]. In both methods, by immobilizing an internal control such as actin or G3PDH on a filter or support, differences in the expression of mRNA derived from proliferative glomerulonephritis-related gene between a control specimen and a target specimen can be accurately detected. Further, by synthesizing target cDNA using different NTP which is labeled respectively based on RNA derived from a control specimen and a target specimen, and hybridizing two labeled cDNA probes to 1 filter or 1 support at the same time, expression level of mRNA derived from proliferative glomerulonephritis-related gene can be determined accurately.

In the case of RNase protection assay, a promoter sequence such as T7 promoter or SP6 promoter is bound to the 3′ end of DNA of the present invention, and then labeled antisense RNA is synthesized by using rNTP labeled by in-vitro transcription system using RNA polymerase. The labeled antisense RNA is bound with specimen-derived RNA to form an RNA-RNA hybrid, followed by digesting with Rnase. Then, the RNA fragments protected from digestion is subjected to gel electrophoresis to form a band, and is detected. By quantitatively determining the obtained band, the expression level of mRNA derived from proliferative glomerulonephritis-related gene can be determined.

6. Method for Detecting Causative Gene of Renal Disease

Hereinafter, a method for detecting a causative gene of renal disease by using proliferative glomerulonephritis-related DNA of the present invention is described.

Examples of DNA which can be used in this method include DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159.

The most clear test for assessing the presence of a mutation which is a cause of renal disease in a proliferative glomerulonephritis-related gene locus is to directly compare a gene from a control group with a gene from a renal disease patient.

Specifically, a human biological specimen such as kidney tissue, blood serum, or saliva or a specimen derived from primary culture cells established from this biological specimen is collected from a renal disease patient and a healthy subject, and DNA is extracted from the biological specimen or the specimen derived from primary culture cells (hereinafter, this DNA is referred to as “specimen-derived DNA”). The specimen-derived DNA or proliferative glomerulonephritis-related DNA which was amplified by using a primer designed on the basis of a nucleotide sequence of DNA of the present invention can be used as specimen DNA. As an alternate method, a DNA fragment containing a proliferative glomerulonephritis-related DNA sequence amplified by performing PCR with primers designed on the basis of a nucleotide sequence of DNA of the present invention with employing the specimen-derived cDNA as a template, can be used as specimen DNA.

To detect whether or not a mutation which is a cause of renal disease is present in proliferative glomerulonephritis-related DNA, a method for detecting a hetero double strands which is formed by hybridization of a DNA strand having a wild-type allele with a DNA strand having a mutant allele can be used.

Examples of methods of detecting a hetero double strands include (1) hetero double strands detection by polyacrylamide gel electrophoresis [Trends Genet., 7, 5 (1991)], (2) single strand conformation polymorphism analysis [Genomics, 16, 325-332 (1993)], (3) chemical cleavage of mismatches (CCM) [Human Molecular Genetics (1996)], Tom Strachan and Andrew P. Read (BIOS Scientific Publishers Limited), (4) enzymatic cleavage of mismatches [Nature Genetics, 9, 103-104 (1996)], and (5) denaturant gradient gel electrophoresis [Mutat. Res., 288, 103-112 (1993)].

By employing specimen-derived DNA or specimen-derived cDNA as a template and using primers designed based on a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159, proliferative glomerulonephritis-related DNA is amplified as fragments smaller than 200 bp, and subjected to polyacrylamide gel electrophoresis. When hetero double strands is formed by a mutation of proliferative glomerulonephritis-related DNA, mobility is slower than a homo double strands having no mutation, and they can be detected as an additional band. Separation is improved when using a specially prepared gel (Hydro-link, MDE, or the like). When conducting a search of fragments smaller than 200 bp, insertion, deletion, and most single nucleotide substitutions can be detected. It is preferable to perform hetero double strands analysis on one sheet of gel in combination with single strand conformation polymorphism analysis described below.

In single strand conformation polymorphism analysis (SSCP analysis), proliferative glomerulonephritis-related DNA is amplified as fragments smaller than 200 bp by using specimen-derived DNA or specimen-derived cDNA as a template and using primers designed based on a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159, and the DNA is denatured. Then, the DNA is subjected to electrophoresis in non-denaturing polyacrylamide gel. When performing DNA amplification, the amplified proliferative glomerulonephritis-related DNA can be detected as a band by labeling the primer with a radioisotope or fluorescent dye, or alternatively by silver staining unlabeled amplified products. In order to clarify the differences with a wild-type pattern, the control specimen is subjected to electrophoresis at the same time, and thereby fragments having a mutation can be detected by detecting the difference in mobility.

In chemical cleavage of mismatches (CCM), DNA fragments of proliferative glomerulonephritis-related DNA is amplified by employing specimen-derived DNA or specimen-derived cDNA as a template and using primers designed based on a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159. The amplified DNA fragments is hybridized with labeled DNA obtained by incorporating a radioisotope or fluorescent dye into DNA of the present invention, and treated with osmium tetroxide, and thereby one of the strands of DNA is cleaved at a mismatching place to enable detection of mutation. The CCM method is one of the detection methods with the highest sensitivity, and can also be adapted to a specimen of kilobase length.

Instead of the above osmium tetroxide, by combining an enzyme involved in mismatch repairing in a cell such as T4 phage lyzolbase or endonuclease VII, and RNaseA, a mismatch can be enzymatically cleaved.

In denaturing gradient gel electrophoresis (DGGE), DNA fragments of proliferative glomerulonephritis-related DNA is amplified by employing specimen-derived DNA or specimen-derived cDNA as a template and using primers designed based on a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159, and is subjected to electrophoresis using a gel having a concentration gradient of a chemical denaturing agent and temperature gradient of a chemical denaturant. The amplified DNA fragments shift in the gel as far as a position at where the DNA is denatured into a single strand, and stop to shift after denaturation. Because the mobility of amplified DNA in the gel differs between the case where a mutation exists in proliferative glomerulonephritis-related DNA and the case where mutation does not exist, the presence of a mutation can be detected. The detection sensitivity may be raised by adding a poly(G:C) terminal to the respective primers.

Another method of detecting a causative gene of renal disease is the protein truncation test (PTT) [Genomics, 20, 1-4 (1994)]. This test enables specific detection of a frameshift mutation, splice site mutation and nonsense mutation which generate deletion of a polypeptide. In the PTT method, specific primers are designed in which T7 promoter sequence and a translation initiation sequence of eukaryote are connected to the 5′ end of DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159, and cDNA is prepared from specimen-derived RNA by reverse transcription PCR (RT-PCR) using the primers. When in vitro transcription and translation is performed using the cDNA, a polypeptide is produced. The polypeptide is subjected to gel electrophoresis, and if the migration position of the polypeptide is a position equivalent to that of a full length polypeptide, there is no mutation which generates a deletion. If the polypeptide has a deletion, the polypeptide migrates to a position shorter than that of a full length polypeptide, and from the position the extent of deletion can be detected.

In order to determine a nucleotide sequence of specimen-derived DNA or specimen-derived cDNA, a primer designed on the basis of a nucleotide sequence of DNA of the present invention can be used. By analyzing the determined nucleotide sequence, it can be judged whether or not a mutation that is a cause of renal disease is present in the specimen-derived DNA or specimen-derived cDNA.

A mutation present in the region other than the coding region of the proliferative glomerulonephritis-related gene can be detected by examining a non-coding region such as the vicinity of the gene or an intron and regulating sequence therein. Renal disease caused by mutation in a non-coding region can be confirmed by detecting mRNA of an abnormal size or of an abnormal production amount in a renal disease patient when compared with a control specimen in accordance with the method described above.

The gene where the presence of a mutation in a non-coding region is suggested, can be cloned by using, as hybridization probe, DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159. The mutation in the non-coding region can be searched in accordance with any of the aforementioned methods.

By conducting statistical processing according to a method described in Handbook of Human Genetics Linkage (The John Hopkins University Press, Baltimore (1994)), the discovered mutation can be identified as an SNPs (single nucleotide polymorphism) which is linked to a renal disease. Further, by obtaining DNA from a family having a clinical history of renal disease by the previously shown method and detecting a mutation, a causative gene of renal disease can be identified.

7. Method for Diagnosing Onset Probability and Prognosis of Renal Disease Using Proliferative Glomerulonephritis-Related DNA

Examples of DNA that can be used in this method include DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159, as well as DNA fragments obtained from these DNA.

A cause of renal disease can be confirmed by detecting a mutation of a gene in any tissue of a human. For example, when a mutation exists in a germ line, there is a probability that the individual which inherited the mutation is prone to develop renal disease. The mutation can be detected by examining DNA from any tissue of the body of the individual. For example, renal disease can be diagnosed by collecting blood, extracting DNA from cells of the blood, and testing for a gene mutation using the DNA. Further, antenatal diagnosis can be performed by testing for a gene mutation using embryonic cells, placental cells or amniocyte.

Further, by testing DNA obtained from tissue of a lesion site of a patient affected by renal disease, the results can be utilized to, for example, diagnose the type of the renal disease and select a medicament to be administered. To detect a gene mutation in tissue, it is useful to isolate lesion site tissue released from surrounding normal tissue. Kidney of a renal disease patient can be extracted by means of biopsy. The tissue obtained in this manner is treated with trypsin or the like, and the obtained cells are cultured in an adequate medium. Chromosome DNA and mRNA can be extracted from the cultured cells.

Hereinafter, DNA obtained from a human specimen by any of the aforementioned methods for the purpose of diagnosis is referred to as “diagnostic specimen-derived DNA”. Further, cDNA synthesized from RNA obtained from a human specimen by any of the aforementioned methods for the purpose of diagnosis is referred to as “diagnostic specimen-derived cDNA”.

Diagnosis of a renal disease can be performed by a method in accordance with the above-described method for detecting a causative gene of renal disease using proliferative glomerulonephritis-related DNA and diagnostic specimen-derived DNA or diagnostic specimen-derived cDNA.

Further, in the diagnosis of a renal disease using proliferative glomerulonephritis-related DNA and diagnostic specimen-derived DNA or diagnostic specimen-derived cDNA, there can be used methods such as (1) detecting a restriction enzyme site, (2) a method using an allele specific oligonucleotide probe (ASO: allele specific oligonucleotide hybridization), (3) PCR using an allele specific oligonucleotide (ARMS: amplification refractory mutation system), (4) oligonucleotide ligation assay (OLA), (5) PCR-preferential homoduplex formation assay (PCR-PHFA), and (6) a method using oligo DNA array [Tanpakushitsu Kakusan Koso (Protein Nucleic Acid Enzyme), 43, 2004-2011 (1998)].

When a restriction site is eliminated or generated by a single nucleotide modification, mutation can be simply detected by amplifying diagnostic specimen-derived DNA or diagnostic specimen-derived cDNA with primers designed based on the sequence of DNA of the present invention, digesting it with the restriction enzyme, and then comparing the obtained restriction enzyme-cleaved DNA fragment with that in the case of a normal person. However, since a single nucleotide modification rarely occurs, for diagnostic purposes, an oligonucleotide probe is designed based on a combination of sequence information about DNA of the present invention and separately-identified mutation information, and mutation is detected by reverse dot blotting where the oligonucleotide probe is bound to a filter and hybridization is carried out.

A short synthetic DNA probe hybridizes only with a completely matched sequence. Therefore, utilizing this characteristic, a single nucleotide mutation can be easily detected by using an allele specific oligonucleotide probe (ASO). For diagnostic purposes, there is often used a reverse dot blotting where an oligonucleotide designed based on the sequence of DNA of the present invention and the identified mutation is bound to a filter, and hybridization is carried out by using a probe prepared by PCR using primers designed based on a sequence of DNA of the present invention from diagnostic specimen-derived DNA or diagnostic specimen-derived cDNA and labeled dNTP. The DNA chip method, where a high-density array is prepared by synthesizing an oligonucleotide designed based on the sequence of DNA of the present invention and the mutation directly onto a support such as a slide glass or silicon, is a mutation detection method suitable for large-scale diagnostic purposes, since a variety mutations can be more easily detected for a small amount of diagnostic specimen-derived DNA or diagnostic specimen-derived cDNA.

A nucleotide mutation can also be detected by oligonucleotide ligation assay (OLA) described below.

Two oligonucleotides having about 20 nucleotides based on the sequence of NA of the present invention, which hybridize to both sides of a mutation site, are prepared. Using diagnostic specimen-derived DNA or diagnostic specimen-derived cDNA as a template and using primers designed based on the sequence of proliferative omerulonephritis-related DNA, proliferative glomerulonephritis-related DNA fragments is amplified by PCR. The amplified fragments are hybridized to the aforementioned oligonucleotides. After hybridization, the two oligonucleotides are ligated by DNA ligase. Whether or not ligation has occurred can be quickly detected by, for example, attaching biotin to one of the oligonucleotides and a different label such as digoxigenin to the other oligonucleotide. Since electrophoresis or centrifugation operation is not necessary in OLA, OLA is a mutation detection method suitable for effectively diagnosing a large number of samples in a short time.

Further, small amounts of mutated gene can be quantitatively and simply detected by the PCR-PHFA method described below.

The PCR-PHFA method is a combination of 3 methods: polymerase chain reaction (PCR), hybridization in liquid phase which shows extremely high specificity, and ED-PCR (enzymatic detection of PCR product) which detects PCR product by an operation similar to ELISA. PCR is performed using DNA of the present invention as a template and using a primer set labeled with dinitrophenyl (DNP) and biotin, to thereby prepare amplified products labeled at both ends. To these are mixed 20- to 100-fold excess amount of unlabeled amplified products obtained by performing amplification using a primer set having the same sequence but without labels and employing diagnostic specimen-derived DNA or diagnostic specimen-derived cDNA as a template. Then, after heat denaturation, the mixture is cooled at a moderate temperature gradient of about 1° C./5-10 minutes, to preferentially form a complete complementary strand. The thus reconstituted labeled DNA is captured and adsorbed in streptavidin immobilized wells via biotin, and is then detected by coloring reaction with an enzyme by binding it with an enzyme-labeled anti-DNP antibody via DNP. If gene having the same sequence as labeled DNA is not present in the specimen, the original double-stranded labeled DNA is reconstituted preferentially and exhibits coloring. In contrast, if a gene of the same sequence is present, because the amount of reconstituted labeled DNA decreases since substitution of complementary strand occurs at random, coloring decreases remarkably. Thus, detection and quantitative determination of a known mutation and polymorphic gene become possible.

8. Method of Immunological Detection and Ouantitative Determination of Proliferative Glomerulonephritis-Related Polypeptide Using Antibody which Specifically Recognizes Proliferative Glomerulonephritis-Related Polypeptide

Examples of a method for immunological detection and quantitative determination of a microorganism, animal cell or insect cell or tissue which expresses a proliferative glomerulonephritis-related polypeptide intracellularly or extracellularly using an antibody (polyclonal antibody or monoclonal antibody) which specifically recognizes a proliferative glomerulonephritis-related polypeptide of the present invention include a fluorescent antibody method, enzyme linked immunosorbent assay (ELISA), a radioimmunoassay (RIA), immunohistochemical staining methods such as immunohistological staining and immunological cell staining (ABC method, CSA method, and the like), western blotting, dot blotting, immunoprecipitation, and sandwich ELISA[Experimental Manual for Monoclonal Antibodies (in Japanese) (Kodansha Scientific) (1987)], Biochemical Experimental Lectures (Second Series) 5, Methods of Immunobiochemical Investigation (In Japanese) (Tokyo Kagaku Dojin (1986)).

In the fluorescent antibody method, an antibody of the present invention is reacted with a microorganism, animal cell or insect cell or tissue which expresses a proliferative glomerulonephritis-related polypeptide intracellularly or extracellularly, and an anti-mouse IgG antibody labeled with a fluorescent substance such as fluorescein isothiocyanate (FITC) or a fragment thereof is further reacted, and then fluorochrome is measured by a flow cytometer.

In the enzyme linked immunosorbent assay (ELISA), an antibody of the present invention is reacted with a microorganism, animal cell or insect cell or tissue which expresses a proliferative glomerulonephritis-related polypeptide intracellularly or extracellularly, and an anti-mouse IgG antibody or bonded fragment labeled with an enzyme such as peroxidase, or biotin is further reacted, and then coloring dye is measured by an absorptiometer.

In the radioimmuno assay (RIA), an antibody of the present invention is reacted with a microorganism, animal cell or insect cell or tissue which expresses a proliferative glomerulonephritis-related polypeptide intracellularly or extracellularly, and an anti-mouse IgG antibody labeled with a radioactive label or a fragment thereof is further reacted, and then measurement is conducted using a scintillation counter or the like.

In the immunological cell staining and the immunohistological staining, an antibody which specifically recognizes a proliferative glomerulonephritis-related polypeptide is reacted with a microorganism, animal cell or insect cell or tissue which expresses a proliferative glomerulonephritis-related polypeptide intracellularly or extracellularly, and an anti-mouse IgG antibody or a fragment thereof labeled with a fluorescent substance such as FITC or an enzyme such as peroxidase, or biotin is further reacted, and then observation is carried out using a microscope.

In the western blotting method, extract of a microorganism, animal cell or insect cell or tissue which expresses a proliferative glomerulonephritis-related polypeptide intracellularly or extracellularly is fractionated by SDS-polyacrylamide gel electrophoresis [Antibodies-A Laboratory Manual, Cold Spring Harbor Laboratory, (1988)], and the gel is blotted on a PVDF membrane or nitrocellulose membrane. Then, an antibody which specifically recognizes a proliferative glomerulonephritis-related polypeptide of the present invention is reacted with this membrane, and further an anti-mouse IgG antibody or a flagment thereof labeled with a fluorescent substance such as FITC or an enzyme such as peroxidase, or biotin is reacted, and the detection is carried out.

In the dot blotting method, extract of a microorganism, animal cell or insect cell or tissue which expresses a proliferative glomerulonephritis-related polypeptide intracellularly or extracellularly is blotted on a nitrocellulose membrane, and an antibody of the present invention is reacted with the membrane, and then an anti-mouse IgG antibody or bonded fragment labeled with a fluorescent substance such as FITC or an enzyme such as peroxidase, or biotin is further reacted, and the detection is carried out.

In the immunoprecipitation, extract of a microorganism, animal cell or insect cell or tissue which expresses a polypeptide of the present invention intracellularly or extracellularly is reacted with an antibody which specifically recognizes a proliferative glomerulonephritis-related polypeptide of the present invention, and then a support having a specific binding ability to immunoglobulin, such as protein G-Sepharose, is added, and thereby an antigen-antibody complex is precipitated.

In the sandwich ELISA method, two types of antibodies having different antigen recognition site, which specifically recognize a proliferative glomerulonephritis-related polypeptide of the present invention, are used. One antibody is adsorbed on a plate beforehand, and the other antibody is labeled with a fluorescent substance such as FITC or an enzyme such as peroxidase, or biotin. Then, extract of a microorganism, animal cell or insect cell or tissue which expresses a proliferative glomerulonephritis-related polypeptide intracellularly or extracellularly is reacted with the antibody adsorbed plate, and the labeled antibody is reacted, and the reaction suitable for the label is carried out.

9. Method of Diagnosing Renal Disease Using Antibody which Specfically Recognizes Proliferative Glomerulonephritis Related Polypeptide

Identification of changes in an expression level of a proliferative glomerulonephritis related polypeptide and structural changes of an expressing polypeptide in a human biological specimen and human primary culture cells is useful from the viewpoint of understanding the risk of onset of renal disease in the future or a cause of a renal disease that has already developed.

Methods of detecting and diagnosing expression level and structural changes of a proliferative glomerulonephritis related polypeptide include the fluorescent antibody method, enzyme linked immuno sorbent assay (ELISA), radioimmuno assay method (RIA), immunohistochemical staining such as immunohistological staining and immunological cell staining (ABC method, CSA method, and the like), western blotting, dot blotting, immunoprecipitation, and sandwich ELISA method, as mentioned above.

As a specimen to be subjected to the diagnosis according to the above methods, a biological specimen itself obtained from the patient such as tissue of the lesion site of the renal disease, blood, blood serum, urine, stool or saliva, or cells or cell extract obtained from the biological specimen is used. Further, tissue obtained from a biological specimen is isolated as a paraffin or cryostat section, can be used.

10. Method for Screening a Therapeutic Agent for Renal Disease Using Proliferative Glomerulonephritis-Related Polypeptide, DNA Encoding the Polypeptide, or Antibody Recognizing the Polypeptide

Examples of DNA which can be used in this screening method include DNA having a nucleotide sequence shown by SEQ ID NO: 1, 3, 5, 7, 9, 11, 13, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 157 or 159. Examples of a polypeptide which can be used include a polypeptide having an amino acid sequence selected from the amino acid sequences shown by SEQ ID NO: 2, 4, 6, 8, 10, 12, 14, 16, 158 and 160, or a polypeptide derived from the amino acid sequence of said polypeptide by deletion, substitution or addition of one or more amino acids and having an activity involved in formation and repair of renal lesion. Examples of an antibody include an antibody which recognizes the polypeptide.

A microorganism, animal cell or insect cell, transformed by introduction of proliferative glomerulonephritis-related DNA of the present invention so as to produce a proliferative glomerulonephritis-related polypeptide or a polypeptide which constitutes a part of a proliferative glomerulonephritis-related polypeptide of the present invention, as well as a purified proliferative glomerulonephritis-related polypeptide or proliferative glomerulonephritis-related polypeptide, are useful for screening an agent which specifically acts on a proliferative glomerulonephritis-related polypeptide. An agent obtained by the screening is useful in treatment of renal disease.

One method of the above-described screening is to select a target compound which specifically binds to a microorganism, animal cell or insect cell transformed so as to produce a proliferative glomerulonephritis-related polypeptide or a polypeptide which constitutes a part of a proliferative glomerulonephritis-related polypeptide of the present invention (hereinafter referred to as an “exploratory transformant”). By performing comparison with a control group of microorganisms, animal cells or insect cells which have not been transformed, the specific target compound can be detected. Further, competitive screening of a target compound can be performed by using, as an index, inhibition of binding of the compound or polypeptide which binds specifically to the exploratory transformant.

A purified proliferative glomerulonephritis-related polypeptide of the present invention or a polypeptide which constitutes a part of the proliferative glomerulonephritis-related polypeptide, can be used to select a target compound which specifically binds to a proliferative glomerulonephritis-related polypeptide. Quantitative determination of a target compound can be performed by an immunological method described above using an antibody which specifically recognizes a proliferative glomerulonephritis-related polypeptide of the present invention. Further, competitive screening of a target compound can be performed by using, as an index, inhibition of binding of a proliferative glomerulonephritis-related polypeptide or a compound which binds to the proliferative glomerulonephritis-related polypeptide.

Another example of a method for the above-described screening is a method in which many peptides constituting a part of a proliferative glomerulonephritis-related polypeptide are synthesized at high density on a plastic pin or a certain type of solid support, and a polypeptide or compound which selectively binds to the peptides is efficiently screened (WO84/03564).

In a kidney-derived cell strain, an agent for regulating expression which promotes expression of mRNA derived from proliferative glomerulonephritis-related gene or proliferative glomerulonephritis-related polypeptide is also useful in the treatment of renal disease.

By adding various test compounds to a kidney-derived cell line and assaying an increase or decrease in expression of mRNA derived from proliferative glomerulonephritis related gene using proliferative glomerulonephritis-related DNA of the present invention, a compound which suppresses or promotes transcription or translation of the proliferative glomerulonephritis related gene can be screened. The increase or decrease in expression of mRNA derived from proliferative glomerulonephritis-related gene can be detected by the above-described PCR, northern blotting, or RAase protection assay methods.

By adding various test compounds to a kidney-derived cell line and assaying an increase or decrease in expression of a proliferative glomerulonephritis-related polypeptide using an antibody which specifically recognizes a proliferative glomerulonephritis-related polypeptide of the present invention, a compound which promotes transcription or translation of a proliferative glomerulonephritis-related gene can be screened. The increase or decrease in expression of a proliferative glomerulonephritis-related polypeptide can be detected by radioimmuno assay (RIA), immunohistochemical staining such as immunohistological staining and immunological cell staining (ABC method, CSA method, and the like), western blotting, dot blotting, immunoprecipitation, and sandwich ELISA, as mentioned above.

By administering a compound obtained by the above method as an agent to a renal disease model animal such as Thy-1 nephritis rat, anti-GBM nephritis, serum sickness type nephritis, PAN nephrosis, daunomycin nephrosis, 5/6 nephrectomized rat, or spontaneous lupus nephritis, and measuring urinary polypeptides or albumin of the animal, the therapeutic effect of the compound in a renal disease can be evaluated.

11. Method for Delivering Drug Specifically to Kidney Using Antibody which Specifically Recognizes Proliferative Glomerulonephritis-Related Polypeptide (Drug Delivery Method)

An antibody which can be used in this drug delivery method may be any antibody which recognizes a proliferative glomerulonephritis-related polypeptide of the present invention, but in particular, it is desirable to use a humanized antibody.

Examples of a humanized antibody include human-type chimeric antibody, and human-type CDR (Complementary Determining Region, hereinafter referred to as “CDR”) transplantation antibody.

Human-type chimeric antibody means an antibody comprising an antibody heavy chain variable domain (hereinafter also referred to as “HV” or “VH,” with “heavy chain” referred to as “H chain” and “variable region” referred to as “V region”) and antibody light chain variable region (hereinafter also referred to as “LV” or “VL,” with “light chain” referred to as “L chain”) of a non-human animal, and heavy chain constant region (hereinafter also referred to as “CH,” with “constant region” referred to as “C region”) of a human antibody and light chain constant region (hereinafter also referred to as “CL”) of a human antibody. Any animal such as mouse, rat, hamster or rabbit can be used as a non-human animal, as long as it is possible to prepare a monoclonal antibody producing hybridoma.

A human-type chimeric antibody of the present invention can be produced by obtaining cDNA encoding VH and VL from a hybridoma which produces a monoclonal antibody which binds to a proliferative glomerulonephritis related polypeptide and neutralizes the action of the polypeptide of the present invention, inserting each cDNA into expression vectors for animal cell having a gene encoding human antibody CH and human antibody CL to construct a human-type chimeric antibody recombinant vector, and introducing and expressing the vector in an animal cell.

A CH of human-type chimeric antibody may be any of those belonging to human immunoglobulin (hereinafter referred to as “hIg”), and those of hIgG class are preferable, and any of subclasses hIgG1, hIgG2, hIgG3 and hIgG4 belonging to hIgG class can be used. Further, a CL of human-type chimeric antibody may be any of those belonging to hIg, and those of κ class or λ class can be used.

“Human-type CDR-grafted antibody” means an antibody in which an amino acid sequence of CDR of VH and VL of an antibody of a non-human animal was transplanted into a suitable position of VH and VL of a human antibody.

The human-type CDR-grafted antibody of the present invention can be produced by; constructing cDNA encoding V regions in which CDR sequences of VH and VL of any human antibody have been respectively substituted with CDR sequences of VH and VL of an antibody of a non-human animal which reacts with the proliferative glomerulonephritis-related polypeptide of the present invention, binds to the proliferative glomerulonephritis-related polypeptide of the present invention, and neutralizes an action of the proliferative glomerulonephritis-related polypeptide of the present invention; inserting the respective cDNA into an expression vector for animal cell having a gene encoding CH of a human antibody and CL of a human antibody to construct a human-type CDR-grafted antibody recombinant vector; and introducing and expressing the vector in an animal cell.

A CH of human-type CDR-grafted antibody may be any of those belonging to hlg, and those of hIgG class are preferable, and any of subclasses hIgG1, hIgG2, hIgG3 and hIgG4 belonging to hIgG class can be used. Further, a CL of human-type CDR-grafted antibody may be any of those belonging to hIg, and those of κ class or λ class can be used.

“Human antibody” originally means an antibody naturally present in the human body, but also includes an antibody obtained from a human antibody phage library or human antibody-producing transgenic animal produced based on recent advances in genetic engineering, cellular engineering and developmental engineering techniques.

An antibody present in the human body can be obtained, for example, by the following method.

Human peripheral blood lymphocytes are isolated, and are immortalized by infecting them with EB virus or the like, and then are cloned. The obtained lymphocytes producing the antibody of interest are cultured to obtain the antibody from the culture product.

A human antibody phage library is a library in which antibody fragments such as Fab or single chain antibody are expressed on a phage surface by inserting an antibody gene prepared from human B cell into a phage gene. From this library, by using binding activity to a substrate on which antigen is immobilized as an index, the phage which expresses an antibody fragment having a antigen binding activity of interest can be recovered. The antibody fragment can be further converted into a complete type human antibody by a genetic engineering technique.

“Human antibody-producing transgenic animal” means an animal in which a human antibody gene has been intracellularly incorporated. Specifically, a human antibody-producing transgenic animal can be produced by introducing a human antibody gene into a mouse ES cell, transplanting the ES cell to an early embryo of another mouse, and then developing it. Examples of a method of producing human antibody from a human antibody-producing transgenic animal include a method of producing and accumulating human antibody in culture product by obtaining and culturing a human antibody-producing hybridoma by a hybridoma production method which is conventionally performed in a non-human mammalian.

Examples of an antibody fragment include Fab, Fab′, F(ab′) 2, single strand antibody, disulfide stabilized V region fragment (hereinafter also referred to as “dsFv”), and peptide including CDR.

Fab is an antibody fragment having a molecular weight of approximately 50,000 and antigen binding activity wherein, among the fragments obtained by treatment of IgG with protease papain (cleaved at 224th amino acid residue of H chain), approximately half of the N-terminus side of H chain and all of L chain are bonded by disulfide bond.

Fab of the present invention can be obtained by treating an antibody which specifically reacts with a polypeptide of the present invention with protease papain. Further, Fab can be obtained by inserting DNA encoding Fab of the antibody into an expression vector for a prokaryote or an expression vector for a eukaryote, introducing the vector into a prokaryote or eukaryote, and expressing the DNA.

F(ab′) 2 is an antibody fragment having a molecular weight of approximately 100,000 and antigen binding activity, which is slightly larger fragment than those obtained by binding Fab via disulfide binding of hinge region among the fragments obtained by treatment of IgG with protease pepsin (cleaved at 234th amino acid residue of H chain).

F(ab′) 2 of the present invention can be obtained by treating an antibody which specifically reacts with the polypeptide of the present invention with protease pepsin. Further, Fab′ can be acquired by inserting DNA encoding F(ab′) 2 of the antibody into an expression vector for a prokaryote or an expression vector for a eukaryote, introducing the vector into a prokaryote or eukaryote, and expressing the DNA.

Fab′ is an antibody fragment having a molecular weight of approximately 50,000 and antigen binding activity which is obtained by cleaving disulfide binding of hinge region of the above-described F(ab′) 2.

Fab′ of the present invention can be obtained by treating an antibody which specifically reacts with a polypeptide of the present invention with a reducing agent, dithiothreitol. Further, Fab′ can be obtained by inserting DNA encoding Fab′ fragment of the antibody into an expression vector for a prokaryote or an expression vector for a eukaryote, introducing the vector into a prokaryote or eukaryote, and expressing the DNA.

“Single chain antibody” (hereinafter also referred to as “scFv”) means a VH-P-VL or VL-P-VH polypeptide obtained by connecting a single chain VH and single chain VL using an adequate peptide linker (herein referred to as “P”). For VH and VL contained in scFv used in the present invention, an antibody which specifically reacts with a polypeptide of the present invention, for example, an antibody derived from humanized antibody or human antibody, can be used.

Single chain antibody of the present invention can be obtained by the following method.

cDNA encoding VH and VL of an antibody which specifically reacts with a polypeptide of the present invention is obtained, and then DNA encoding single chain antibody is constructed. The DNA is inserted into an expression vector for a prokaryote or an expression vector for a eukaryote, and the recombinant vector is introduced into a prokaryote or eukaryote to express the DNA. Thus, single chain antibody can be obtained.

“Disulfide-stabilized V region fragment” (dsFv) means a fragment obtained by binding the polypeptides wherein 1 amino acid residue in VH and VL is substituted with a cysteine residue via disulfide binding between the cysteine residues. An amino acid residue to be substituted with a cysteine residue can be selected based on the prediction of the steric structure of an antibody in accordance with a method shown by Reiter et al. [Protein Engineering, 7, 697 (1994)]. For VH and VL contained in dsFv used in the present invention, an antibody which specifically reacts with the polypeptide of the present invention, for example, an antibody derived from humanized antibody or human antibody, can be used.

Disulfide-stabilized V region fragment (dsFv) of the present invention can be obtained by the following method.

cDNA encoding VH and VL of an antibody which specifically reacts with the polypeptide of the present invention is obtained, and then DNA encoding dsFv is constructed. The DNA is inserted into an expression vector for a prokaryote or an expression vector for a eukaryote. Then, the recombinant vector is introduced into a prokaryote or eukaryote, and the DNA is expressed to obtain dsFv.

A peptide containing CDR can be prepared by a chemical synthesis method such as an Fmoc method or tBoc method.

A fusion antibody described below which is prepared by using an antibody of the present invention can be used in drug delivery for transporting an agent or protein to a renal lesion.

“Fusion antibody” means an antibody in which an agent such as a radioisotope, polypeptide or low molecular weight compound or the like is bonded chemically or by genetic engineering to an antibody which specifically reacts with a polypeptide of the present invention, for example, a humanized antibody, a human antibody or an antibody fragment thereof.

The fusion antibody of the present invention can be produced by binding chemically or by genetic engineering, an agent such as a radioisotope, polypeptide or low molecular weight compound or the like to the N terminus side or C terminus side of an H chain or L chain of an antibody which specifically reacts with the polypeptide of the present invention or an antibody fragment thereof, to an adequate substituent or side chain in the antibody or antibody fragment, or to a sugar chain in the antibody or antibody fragment.

Examples of a radioisotope include ¹³¹I and ¹²⁵I, which can be bound to an antibody or antibody fragment by a chloramine-T method or the like.

Examples of a low molecular weight compound include alkylating agents such as nitrogen mustard and cyclophosphamide; antimetabolites such as 5-fluorouracil and methotrexate; antibiotics such as daunomycin, bleomycin, mitomycin C, daunorubicin and doxorubicin; anticancer agents including plant alkaloids such as vincristine, vinblastine and vindesine, and hormone agents such as tamoxifen and dexamethasone [Clinical Oncology (in Japanese) (Japanese Clinical Tumor Society Edition, 1996, Cancer and Chemotherapy Co.)]; as well as steroid drugs such as hydrocortisone and prednisone; nonsteroid drugs such as aspirin and indomethacin; immunomodulators such as gold thiomalate and penicillamine; immunosuppressors such as cyclophosphamide and azathioprine; and anti-inflammatory agents such as antihistamines like chlorpheniramine maleate and clemastine [Inflammation and Antiinflammation Therapy (in Japanese) (1982) Ishiyaku Shuppan Kabushiki Kaisha].

A low molecular weight compound can be bonded to the above-described antibody by a standard method. For example, methods for binding daunomycin with an antibody include a method of binding daunomycin with an amino group of an antibody via glutaraldehyde, and a method of binding an amino group of daunomycin and a carboxyl group of an antibody via soluble carbodiimide.

As a polypeptide, cytokines which activates immunocompetent cells or growth controlling factors of vascular endothelium, vascular smooth muscle and the like are preferable, and examples include human interleukin 2, human granulocyte-macrophage colony-stimulating factor, human macrophage colony-stimulating factor, human interleukin 12, fibroblast growth factor-2 (FGF-2), and platelet-derived growth factor (PDGF).

A fusion antibody with a polypeptide can be obtained by the following method.

cDNA encoding a polypeptide is ligated to cDNA encoding an antibody or antibody fragment to construct DNA encoding a fusion antibody. The fusion antibody can be obtained by inserting the DNA into an expression vector for a prokaryote or an expression vector for a eukaryote, introducing the recombinant vector into a prokaryote or eukaryote, and expressing the DNA.

12. Therapeutic Agent for Renal Disease which Comprises Proliferative Glomerulonephritis-Related Polypeptide

The proliferative glomerulonephritis-related polypeptide of the present invention can be used for the reconstruction of a structure and function of a kidney in renal disease typically represented by nephritis.

A therapeutic agent for renal disease which comprises a proliferative glomerulonephritis-related polypeptide of the present invention may contain only the polypeptide as an active ingredient, however, it is preferable that the polypeptide is normally mixed together with one or more pharmacologically acceptable carriers and provided as a medical preparation prepared by any method well-known in the technical field of pharmaceutics.

As the route of administration, it is preferred to use the most effective one at the time of treatment, and examples include oral administration, or parenteral administration such as intraoral, tracheobronchial, endorectal, subcutaneous, intramuscular and intravenous administration. Examples of dosage form include a nebula, a capsule, a tablet, a granule, syrup, an emulsion, a suppository, an injection, an ointment, and a tape.

Examples of a suitable preparation for oral administration include an emulsion, syrup, a capsule, a tablet, a powder, and a granule. For example, liquid preparations such as an emulsion or syrup can be produced by using, as an additive, water; sugars such as sucrose, sorbitol and fructose; glycols such as polyethylene glycol and propylene glycol; oils such as benne oil, olive oil and soybean oil; an antiseptic such as p-hydroxybenzoic acid ester; flavors such as strawberry flavor and peppermint; and the like. A capsule, a tablet, a powder, a granule, and the like can be produced by using, as an additive, an excipient such as lactose, glucose, sucrose or mannitol; a disintegrating agent such as starch or sodium alginate; a lubricant such as magnesium stearate or talc; a binding agent such as polyvinyl alcohol, hydroxypropylcellulose or gelatin; a surfactant such as fatty acid ester; a plasticizer such as glycerine; and the like.

Examples of a suitable preparation for parenteral administration include an injection, a suppository and a nebula. For example, an injection is prepared by using a carrier or the like comprising a salt solution, a glucose solution or a mixture thereof. A suppository is prepared by using a carrier comprising theobroma oil, hydrogenated fat, carboxylic acid, or the like. Further, a nebula is prepared by using the polypeptide alone or together with a carrier or the like which can disperse the polypeptide as fine particles and makes the absorption easy without stimulating the oral cavity and mucosa of respiratory tract of a recipient. Specific examples of a carrier include lactose and glycerine. Depending on the properties of the polypeptide and carrier to be used, a formulation such as an aerosol or a dry powder is also possible. Further, the ingredients exemplified above as additives for an oral agent can be added in these parenteral agents.

An administration dose and administration frequency differs depending on the type of the disease, the administration method, the treatment period, the age, the body weight and the like. Normally, a dose for an adult is 10 μg/kg to 8 mg/kg per day.

13. Gene Therapy Agent which Comprises Proliferative Glomerulonephritis-Related DNA

A gene therapy agent using a virus vector containing proliferative glomerulonephritis-related DNA of the present invention can be produced by combining the recombinant virus vector prepared in Item 4. above and a base for use in a gene therapy agent [Nature Genet., 8, 42(1994)].

As a base for use in a gene therapy agent, any base which is normally used in an injection may be used, and examples include distilled water; salt solution such as sodium chloride or a mixture of sodium chloride and an inorganic salt; a sugar solution such as mannitol, lactose, dextran or glucose; an amino acid solution such as glycine or arginine; and a mixture of organic acid solution or salt solution and glucose solution. Further, by using an auxiliary agent such as an osmoregulation agent, a pH adjuster, vegetable oil such as benne oil or soybean oil, or a surfactant such as lecithin or a nonionic surfactant together with the aforementioned bases in accordance with the usual manner, an injection may be prepared as a solution, a suspension, or a dispersion. These injections can also be prepared as formulations to be dissolved before use by an operation such as powderization or lyophillization. The gene therapy agent of the present invention can, in the case of a liquid, be used for treatment as it is, and in the case of a solid, be used after dissolving immediately before gene therapy in the above-described base which has been sterilized as necessary. Examples of an administration method of the gene therapy agent of the present invention include a method of administering locally so as to be absorbed in treatment area of the kidney of the patient.

As a system for transporting a virus vector to a renal lesion more specifically, there is a method of using a fusion protein of a single chain antibody which specifically recognizes BMP-7 receptor and Env protein of a retrovirus vector [Proc. Natl. Acad. Sci. USA, 92, 7570-7574 (1995)]. This system is not limited to a retrovirus vector, and may also be applied to a lentivirus vector and the like.

A virus vector can be prepared by preparing a complex by combining a proliferative glomerulonephritis-related DNA of the present invention of a suitable size with polylysine-conjugate antibody which is specific to adenovirus hexon polypeptide, and then binding the obtained complex to an adenovirus vector. The virus vector stably reaches a target cell, is incorporated into the cell by an endosome, and is decomposed within the cell to efficiently express the gene.

A virus vector based on Sendai virus which is (−)strand RNA virus has also been developed (WO97/16538, WO97/16539), and it is also possible to prepare a Sendai virus vector incorporating proliferative glomerulonephritis DNA for the purpose of gene therapy.

Proliferative glomerulonephritis-related DNA can also be transported to a renal lesion by a non-viral gene transfer technique.

Examples of non-viral gene transfer techniques known in the art include a calcium phosphate coprecipitation [Virology, 52, 456-467 (1973); Science, 209, 1414-1422 (1980)], a microinjection [Proc. Natl. Acad. Sci. USA, 77, 5399-5403 (1980); Proc. Natl. Acad. Sci. USA, 77, 7380-7384 (1980); Cell, 27, 223-231 (1981); Nature, 294, 92-94 (1981)], membrane fusion mediated by liposome [Proc. Natl. Acad. Sci. USA, 84, 7413-7417 (1987); Biochemistry, 28, 9508-9514 (1989); J. Biol. Chem., 264, 12126-12129 (1989); Hum. Gene Ther., 3, 267-275, (1992); Science, 249, 1285-1288 (1990); Circulation, 83, 2007-2011 (1992)], and a direct DNA incorporation and receptor-mediation DNA transfer [Science, 247, 1465-1468 (1990); J. Biol. Chem., 266, 14338-14342 (1991); Proc. Natl. Acad. Sci. USA, 87, 3655-3659 (1991); J. Biol. Chem., 264, 16985-16987 (1989); BioTechniques, 11, 474-485 (1991); Proc. Natl. Acad. Sci. USA, 87, 3410-3414 (1990); Proc. Natl. Acad. Sci. USA, 88, 4255-4259 (1991); Proc. Natl. Acad. Sci. USA, 87, 4033-4037 (1990); Proc. Natl. Acad. Sci. USA, 88, 8850-8854 (1991); Hum. Gene Ther., 3, 147-154 (1991)].

It has been reported in tumor-related research that in the method of transferring gene using membrane fusion mediated by liposome by directly administering a liposome preparation to target tissue, localized incorporation and expression of the gene in the tissue becomes possible [Hum. Gene Ther. 3, 399-410 (1992)]. Therefore, a similar effect can also be expected in a renal lesion. For direct targeting of DNA to a renal lesion, a direct DNA-incorporation technique is preferred. Transferring DNA mediated by receptor is performed, for example, by conjugating DNA (normally, a supercoiled plasmid which is covalently cyclized) to a protein ligand via polylysine. The ligand is selected based on the presence of a corresponding ligand receptor on the cell surface of a target cell or tissue. Examples of a receptor and ligand combination include a combination of BMP-7 receptor and BMP-7. The ligand-DNA conjugate can, as desired, be directly injected into a blood vessel, and can be directed to target tissue in which binding of receptor and internalization of the DNA-polypeptide complex occurs. To prevent intracellular destruction of DNA, it is possible to simultaneously infect with adenovirus and destroy the endosome functions.

14. Therapeutic Agent for Renal Disease which Comprises Antibody which Specifically Recognizes Proliferative Glomerulonephritis-Related Polypeptide

An antibody which specifically recognizes a proliferative glomerulonephritis-related polypeptide of the present invention can be directly utilized in the treatment of diseases such as renal cancer in which cell neogenesis in the kidney is progressing.

A therapeutic agent which comprises an antibody which specifically recognizes a proliferative glomerulonephritis-related polypeptide of the present invention may contain only the antibody as an active ingredient. Normally, it is preferable that the polypeptide is mixed together with one or more pharmacologically acceptable carriers and provided as a medical preparation prepared by any method well-known in the technical field of pharmaceutics. Preparation and administration of the therapeutic agent can be carried out in accordance with the description for the agent containing a proliferative glomerulonephritis-related polypeptide described in 13. above.

Hereinafter, examples of the present invention are described.

### BEST MODE FOR CARRYING OUT THE INVENTION

### EXAMPLE 1

### Preparation of Thy-1 Nephritis Rat Kidney cDNA Library

Anti-rat Thy-1 monoclonal antibody OX-7 (manufactured by Cedarlane) was administered by tail vein injection to 20 Wistar rats (males) (manufactured by Japan SLC Co., body weight; approximately 200 g) at a dose of 1 mg/kg, thereby inducing nephritis. Physiological saline was administered to a control group. For these rats, the polypeptide concentration in urine was measured using urine analysis stick pretest 6B (manufactured by Wako Pure Chemical Industries), and was used as an index of nephritis condition.

On each of days 2, 4, 6, 8, 10, 13 and 16 after administration of OX-7, kidney was extracted from 3 rats (on day 16 only, from 2 rats), as well as from rats of the control group, and total RNA was extracted from each individual by the guanidine thiocyanate-trifluoroacetic acid cesium technique [Methods in Enzymology, 154, 3 (1987)]. For the control group, total RNA was not extracted from each individual, and instead total RNA was extracted from a mixture comprising equal amounts of renal tissue lysate prepared by treating the kidneys of 3 individuals on day 2 after administration of physiological saline and renal tissue lysate prepared by treating the kidneys of 3 individuals on day 10 after administration. Of these total RNAs, the total RNA of each individual rat kidney of days 2, 4, 6, 8 and 10 after OX-7 administration were mixed together in equal amounts, and poly (A) RNA was prepared by using oligo(dT) cellulose. Then, using ZAP-cDNA Synthesis Kit (manufactured by Stratagene), a cDNA library (total independent plaques of 1.0×10⁶) was prepared (Thy-1 nephritis rat kidney cDNA library). Details of the method for preparing the cDNA are as described in the kit manual. This cDNA library is inserted between the Xho I/EcoR I sites of the vector in such a way that the 5′ end of cDNA was on the EcoR I site side, with using λ phage vector λ ZAP II (manufactured by Stratagene) as a vector.

### EXAMPLE 2

### Preparation of Subtracted Library

(1) Preparation of Single Strand DNA

By infecting host cell Escherichia coli XL1-Blue MRF′ (manufactured by Stratagene) with the Thy-1 nephritis rat kidney cDNA library prepared in Example 1 together with helper phage ExAssist (manufactured by Stratagene) and performing in vivo excision, a phagemid pBluescript SK(−) region containing cDNA was excised from the vector as a single strand DNA phage, and was released into a culture supematant. The method of in vivo excision was performed in accordance with Strategene's manual. 200 μl of this culture supernatant (titer: 8.5×10⁵ cfu/μl) was added to 7 ml of 10 mmol/l MgSO₄ containing 1.8×10¹⁰ Escherichia coli SORL (manufactured by Stratagene) as a host cell which cannot be infected with ExAssist, and incubated at 37° C. for 15 minutes. The total volume was added to 200 ml of 2-fold YT culture medium (1.6% Bacto-tryptone, 1% yeast extract), and the mixture was cultured with shaking for 1 hour at 37° C., and was infected with single strand DNA phage containing cDNA. Ampicillin was added thereto at a concentration of 50 μg/ml, the mixture was again subjected to shaking culture for 1 hour at 37° C. to culture only phage-infected Escherichia coli. The number of the cells was measured by an absorbance of 600 nm, and the result was 4×10¹⁰ cells. Helper phage R408 (manufactured by Stratagene) was added at multiplicity of infection (moi)=13(5.3×10¹¹ pfu), and the mixture was cultured with shaking for 7 hours at 37° C. to release single strand DNA again in the supernatant. The culture medium was transferred to a sterile tube, centrifuged for 10 minutes at 10,000 rpm at 4° C., and only supernatant containing phage was transferred and recovered in a new sterile tube. After centrifugation of this supernatant under the same conditions, the cells were completely removed by passing through a sterile filter of 0.22 mm pore size (manufactured by Millipore). 20 ml of 10-fold buffer [100 mmol/l Tris-HCl (pH 7.5), 100 mmol/l MgCl₂] and 140 units of deoxyribonuclease I (manufactured by Nippon Gene) were added, and the mixture was reacted at 37° C. for 30 minutes. 2.5 mol/l NaCl solution (1/4 volume of the reacted mixture) of 20% polyethyleneglycol (molecular weight 6,000) was added thereto, and the mixture was mixed well at room temperature and allowed to stand for 20 minutes, and then centrifuged for 10 minutes at 10,000 rpm at 4° C. to precipitate phage. The supernatant was completely removed, and the obtained phage precipitate was dissolved in 400 μl of TE [10 mmol/l Tris-HCl (pH 8.0), 1 mmol/l EDTA (pH 8.0)]. Then, 4 μl of 10% SDS and 625 μg (25 μl) of proteinase K were added thereto, and the mixture was allowed to react for 1 hour at 42° C. After phenol extraction, phenol-chloroform extraction and chloroform extraction, the aqueous layer was fractionated, then ethanol precipitation was performed to obtain 77.6 μg of single strand DNA [vector pBluescript SK(−)] of Thy-1 nephritis rat kidney cDNA library.

(2) Biotinylation of RNA

From 1.2 mg of total RNA prepared from kidney of control group rat of Example 1, 20 μg of poly(A) RNA was prepared by using oligo(dT) cellulose. To 10 μg of it was added distilled water to bring to 20 μl in a test tube, and 30 μg (30 μl) of 1 mg/ml PHOTOPROBE biotin (manufactured by Vector Laboratories) was added thereto in a dark place. The cap of the test tube was opened and the tube was placed on ice, and RNA was biotinylated by light irradiation for 20 minutes using a mercury lamp from a height of about 10 cm. After addition of 50 μl of a solution of 100 mmol/l 1 Tris-HCl (pH 9.5) and 1 mmol/l EDTA (pH 8.0) to the reaction solution, extraction with water saturated butanol was performed 3 times, and chloroform extraction was performed twice, and then the aqueous layer was precipitated with ethanol. The recovered precipitate of RNA was dissolved in 20 μl of distilled water, the above-described biotinylation reaction operation (operation from addition of PHOTOPROBE biotin to precipitation with ethanol) was performed once more to obtain biotinylated RNA.

(3) Subtraction

To 0.5 μg (1 μl) of single strand DNA of the Thy-1 nephritis rat kidney cDNA library prepared in (1) was added 12.5 μl of 2× hybridization buffer [80% formamide, 100 mmol/l HEPES (pH 7.5), 2 mmol/l EDTA (pH 8.0), 0.2% SDS], 2.5 μl of 2.5 mol/l NaCl, and 1 μg (1 μl) of poly(A) (manufactured by Amersham Pharmacia Biotech), and then 8 μl of biotinylated RNA (RNA 10 μg) prepared in (2) was dissolved in distilled water and added. After the mixture was heated at 65° C. for 10 minutes, hybridization was performed at 42° C. for 63 hours.

To the solution after hybridization reaction was added 400 μl of buffer [500 mmol/l NaCl, 50 mmol/l HEPES (pH 7.5), 2 mmol/l EDTA (pH 8.0)], and 10 μg (5 μl) of streptavidin (manufactured by Life Technologies) was then added thereto, and the mixture was allowed to react at room temperature for 5 minutes. Phenol-chloroform extraction was performed and a complex of streptavidin-biotinylated RNA-hybridized cDNA was removed from the aqueous layer. 10 μg of streptavidin was again added to the aqueous layer and allowed to react at room temperature for 5 minutes, and phenol-chloroform extraction was performed twice, after which chloroform extraction was performed and the aqueous layer recovered. After passing the aqueous layer through Unit Filter Ultra Free C3 Plus TK (manufactured by Millipore) to allow cDNA be adsorbed in the filter, and the filter was washed. Then, concentrated and desalted cDNA was recovered in 30 μl of 1/10 TE [1 mmol/l Tris-HCl (pH8.0), 0.1 mmol/l EDTA (pH 8.0)]. Operations using this filter were performed in accordance with Millipore's manual. By this subtraction operation, cDNA of a gene whose expression level was high in both Thy-1 nephritis rat kidney and control group rat kidney was removed from the cDNA library, and cDNA of a gene which was expressed in Thy-1 nephritis rat kidney but almost not expressed in control group rat kidney was concentrated. However, when using only the above subtraction operation, cDNA of a gene which was expressed at a very low level in Thy-1 nephritis rat kidney but almost not expressed in control group rat kidney would also be concentrated. Therefore, reverse differentiation as described in (5) below was performed, and a library was prepared that did not include cDNA of a gene expressing at a very low level in Thy-1 nephritis rat kidney.

(4) Amplification of cDNA after Subtraction

Since it was considered that the amount of the cDNA had decreased considerably following the subtraction operation of (3), in order to perform the reverse subtraction described in (5) below, the amount was increased in the following manner. 14 μl of distilled water and 2 μg (1 μl) of 5′-AP primer was added to a half amount (15 μl) of the cDNA (single strand DNA) after subtraction. After heating for 10 minutes at 65° C., the mixture was left to stand at room temperature for 5 minutes to anneal the primer to the single strand DNA. 5 μl of 10×Bca BEST reaction buffer [which is attached with BcaBEST Dideoxy Sequencing Kit (manufactured by Takara Shuzo)], 10 μl of 1 mmol/l dNTP (mixture of 1 mmol/l each of dATP, dGTP, dCTP, and TTP), 1.5 μg (0.5 μl) of single strand DNA-binding polypeptide (manufactured by USB), 4 units (2 μl) of BcaBEST DNA polymerase (manufactured by Takara Shuzo) and 2.5 μl of distilled water were added thereto, and the solution was allowed to react for 1 hour at 65° C. to synthesize double strand DNA. 50 μl of distilled water was added to the reaction solution, and phenol-chloroform extraction and chloroform extraction were performed. Then, double stranded DNA was finally recovered in 20 μl of TE by using a Unit Filter Ultra Free C3 Plus TK in the same manner as described in (3).

The total amount of the recovered double strand DNA (4 μl) was introduced into Escherichia coli DH12S (manufactured by Life Technologies) by electroporation. To the Escherichia coli DH12S after the electroporation operation was added 1.5 ml of SOC culture medium, and this was then inoculated into 42.5 ml of LB-Ap culture medium (1% Bacto-tryptone 0.5% yeast extract, 1% NaCl, 50 μg/ml ampicillin). The titer at this stage was 4.3×10⁶ cfu. After culturing at 37° C. for 4 hours, the number of the cells were measured by measuring the absorbance of 600 nm, and the result was 1×10⁸ to 1.5×10⁸ cells/ml. Dimethyl sulfoxide was added to the half amount of the culture at a concentration of 7%, and this was stored at −80° C. The remaining half amount of the culture was infected with helper phage R408 of moi=14-20 (5×10⁸ pfu). After culturing at 37° C. for 15 minutes, each 5 ml of the mixture was inoculated into 5×45 ml of 2-fold YT culture medium, and then cultured at 37° C. 2 hours and 30 minutes after the start of culturing, ampicillin was added at a concentration of 100 μg/ml, and this was then cultured for a further 5 hours and 30 minutes to release single strand DNA phage to the culture solution. In the same manner as (1), 30.8 μg of single strand DNA was purified from this culture solution.

(5) Reverse Subtraction

2.5 μg of poly(A) RNA of Thy-1 nephritis rat kidney prepared in Example 1 was biotinylated in the same manner as in (2). This biotinylated RNA was added to 2.5 μg of the single strand DNA prepared in (4) after subtraction, and distilled water was added thereto to bring to 9 μl. Added thereto was 12.5 μl of 2-fold hibridization buffer which was the same as that used in the subtraction of (3), 2.5 μl of 2.5 mol/l NaCl, and 1 μg (1 μl) of poly(A). After heating the mixture at 65° C. for 10 minutes, hybridization was performed at 42° C. for 59 hours.

Streptavidin was reacted with the solution after hybridization reaction in the same manner as in the differentiation of (3). Phenol-chloroform extraction was performed to remove the aqueous layer, and a phenol-chloroform layer containing a complex of biotinylated RNA derived from Thy-1 nephritis rat kidney and hybridized cDNA was recovered. After repeating 3 times the operation of addition of TE and extraction, TE was again added to the phenol-chloroform layer, and the mixture was heated at 95° C. for 5 minutes, to thereby dissociate the biotinylated RNA and cDNA. After the reaction layer was quenched by immersion in ice water, the solution was stirred vigorously and dissociated cDNA was extracted into an aqueous layer. After heating this solution once more for 5 minutes at 95° C., the quenching and extraction operation was repeated, and an aqueous layer containing dissociated cDNA was recovered by centrifugation. After performing phenol-chloroform extraction and chloroform extraction for the aqueous layer, the aqueous layer was passed through Unit Filter Ultra Free C3 Plus TK in the same manner as in (3). After cDNA was adsorbed in the filter, the filter was washed. Concentration and desalting of cDNA was performed to recover cDNA in 30 μl of 1/10 TE.

(6) Preparation of cDNA Library

For the single strand cDNA obtained in (5) after reverse subtraction, half amount of the cDNA was made into double stranded DNA in the same manner as in (4). ⅛ amount of the double strand DNA was introduced into Escherichia coli DH12S by electroporation to prepare a reverse-subtracted cDNA library. From the analysis using one portion of the library, it was estimated that the number of independent colonies of the library was 2.5×10⁴ cfu and that the ratio of cDNA insertion was 98%.

### EXAMPLE 3

### Differential Hybridization

(1) Preparation of Array Filter

Using the reverse-subtracted cDNA library prepared in (6) of Example 2, colonies were formed on LB-Ap agar medium, and 9,600 colonies among them were inoculated onto 100 96-well plates in which 100 μl of LB-Ap culture medium had been added, at 1 colony/well. After each colony was cultured in the 96-well plates at 37° C., 50 μl of 50% glycerol was added thereto and the colonies were then stored at −80° C. (this storage culture solution is hereinafter referred to as “glycerol stock”).

Onto 96-well plates, each well of which contains 100 μl of LB-Ap culture medium, glycerol stock was again inoculated using 96 pin replicators, and the plates were left to stand for culturing overnight at 37° C. Using an automatic microdispenser, Hydra 96, the culture solution containing Escherichia coli was spotted in spots of 0.5 μl each on nylon membranes in the same lattice formation as the 96-well plate (8 vertically×12 horizontally). On one nylon membrane were spotted 384 colonies in a lattice formation (16 vertically×24 horizontally), which corresponded to the total amount of 4 plates of 96-well plates, and one colony was spotted in the same position on 2 membranes so that 2 of the same membranes could be prepared. Membranes spotted with the culture were placed on LB-Ap agar medium, with the spotted surface upward, and were cultured overnight at 37° C.

After the membranes on which colonies of Escherichia coli had grown sufficiently were stripped from the culture medium, the membranes were placed on paper soaked with denaturing solution for DNA (0.5 mol/l NaOH, 1.5 mol/l NaCl), and left at room temperature for 10 minutes to denature DNA. The membranes were then transferred to paper soaked with neutralizing solution [1.0 mol/l Tris-HCl (pH 7.5), 1.5 mol/l NaCl] and left at room temperature for 10 minutes. After abrasively washing the cell clusters on the membrane in a sufficient amount of 2-fold SSC (0.3 mol/l sodium chloride, 30 mmol/l sodium citrate) containing 0.5% SDS which was prepared in a bath, washing was performed by replacing the same buffer two times. Membranes were transferred to polyethylene bags, a reaction buffer [50 mol/l tris-hydrochloric acid (pH8.5), 50 mol/l EDTA, 100 mol/l sodium chloride, 1% sodium lauroyl sarcosinate] in which proteinase K was dissolved at a concentration of 250 μg/ml was added thereto, and the bags were sealed, and reaction was carried out for 2 hours at 37° C. After the membranes were removed from the bags and washed with 2-fold SSC, the membranes were once again put into polyethylene bags, 2-fold SSC containing proteinase inhibitor Pefabloc (manufactured by Roche) at a concentration of 400 μg/ml was added thereto, and the bags were sealed and treated at room temperature for 1 hour. The membranes were removed from the bags and washed with 2-fold SSC. Finally, DNA was immobilized on the membranes by ultraviolet irradiation using crosslinker optimal link (manufactured by Funakoshi). The thus obtained membranes are referred to as “array filters.”

(2) Preparation of Riboprobe

Using poly(A) RNA derived from Thy-1 nephritis rat kidney and control group rat kidney prepared in Example 1, digoxigenin (DIG) labeled riboprobes were prepared in the following manner. Since the number of membranes is large and a large amount of probes is required, 150 μg of probes is necessary for performing hybridization for 50 membranes of 100 cm². Firstly, double stranded cDNA was prepared from poly(A) RNA, and T7 RNA polymerase reaction was carried out with employing this cDNA as a template, to thereby obtain riboprobes incorporated with DIG

(2)-1 Preparation of Double Stranded cDNA

5 μg of each poly(A) RNA was mixed with 8 μg of T7(dT) primer (SEQ ID NO: 161; having T7 promoter sequence at 5′ end), and distilled water (pure water that was distilled a further 2 times, the same applies hereinafter) was added to bring the mixture to 7.8 μl. The mixture was heated at 70° C. for 10 minutes and was quenched on ice. 4 μl of 5-fold hybridization buffer (a buffer attached with commercially available enzyme), 2 μl of 100 mmol/l DTT and 1.2 μl of 10 mmol/l dNTP was added thereto, and the mixture was mixed well by pipetting. After incubating at 37° C. for 2 minutes to perform annealing, 5 μl of SuperScript II reverse transcriptase (manufactured by Life Technologies) was added, and the reaction was carried out at 42° C. for 1 hour to synthesize single strand cDNA, and then the mixture was cooled with ice.

To the solution after reaction were added 92.3 μl of distilled water, 32 μl of 5-fold hybridization buffer [94 mmol/l tris-hydrochloric acid (pH 6.9), 453 mmol/l potassium chloride, 23 mmol/l magnesium chloride, 750 μmol/l β-NAD, 50 mmol/l ammonium sulfate], 3 μl of 10 mmol/l dNTP, 6 μl of 100 mmol/l DTT, 15 units (2.5 μl) of Escherichia coli DNA ligase (manufactured by Takara Shuzo), 40 units (11.5 μl) of Escherichia coli DNA polymerase I (manufactured by Takara Shuzo), and 1.2 units (2 μl) of Escherichia coli ribonuclease H (manufactured by Takara Shuzo), in that order. After mixing well by pipetting on ice, the mixture was allowed to react at 16° C. for 2 hours and 30 minutes to synthesize double stranded cDNA. The Escherichia coli DNA ligase and Escherichia coli ribonuclease H were used after diluting with a 1-fold reaction buffer immediately before the reaction to dilute the stock solutions to 6 units/μl and 0.6 units/μl, respectively. After reaction, 2 μl of 0.5 mol/l EDTA and 2 μl of 10% SDS were added to stop the reaction, and an aqueous layer was recovered by phenol-chloroform extraction. Then, 70 μl of TE was further added to the phenol-chloroform layer excluding the aqueous layer, and extraction was carried out, and the aqueous layer was combined with the aqueous layer recovered earlier. To this aqueous layer was added 12 μl of proteinase K (2 mg/ml), and this was allowed to react at 42° C. for 1 hour. After recovering an aqueous layer by phenol-chloroform extraction in the solution after reaction, 70 μl of TE was added to the phenol-chloroform layer excluding the aqueous layer and extracted, and the aqueous layer was combined with the aqueous layer recovered earlier.

Using Unit Filter Ultra Free C3LTK (manufactured by Millipore), the aqueous layer was subjected to concentration and desalting. Specifically, the aqueous layer was placed in a filter cup and centrifuged at 8,000 rpm for 5 minutes, thereby adsorbing DNA in the filter. Solution which moved to the lower part was removed, and 300 μl of distilled water was placed in the filter cup, which was again centrifuged at 8,000 rpm for 5 minutes, thereby washing the filter. After repeating this washing operation once more, 25 μl of distilled water was placed in the filter cup, and suspended by pipetting to extract DNA. The filter cup was taken out and inserted upside-down into a tube for centrifugation (Falcon 2059), which was then centrifuged to collect the suspension in the bottom of the tube. 25 μl of distilled water was placed in the filter cup once more, and the suspension was recovered in the same manner (total of 50 μl).

(2)-2 Synthesis and Labeling of RNA

From double stranded cDNA obtained in the above-described manner, DIG labeled riboprobes were prepared using DIG RNA Labeling Kit (manufactured by Roche). The method was in accordance with Roche's DIG System Users Guide. Specifically, 20 μl of a mixture of 1 μg of cDNA (prepared to 14 μl with distilled water), 2 μl of 10× reaction buffer (buffer included in kit), 2 μl of NTP labeling mix (included in kit, and containing DIG-11-UTP) and 2 μl of T7 RNA polymerase (manufactured by Roche) was mixed by pipetting, and then allowed to react at 37° C. for 2 hours. After stopping the reaction by adding 0.8 μl of 0.5 mol/l EDTA, 2.3 μl 4 mol/l lithium chloride (1/9 volume of reaction solution) and 65 μl ethanol (2.5-3 times volume of reaction solution) were added, and the mixture was left at −80° C. for 30 minutes (alternatively, at −20° C. overnight) to precipitate RNA. After centrifugation at 4° C., the supernatant was removed, and the precipitate was washed with 70% ethanol, and air-dried in a clean bench, and was then dissolved in 100 μl distilled water. The yield of synthesized riboprobe was assayed in accordance with Roche's DIG System Users Guide.

(3) Hybridization

The method of hybridization and detection of hybridized spots as well as the reagents were adopted in accordance with Roche's DIG System Users Guide.

To 20 ml of hybridization buffer [5-fold SSC, 0.1% sodium lauroyl sarcosinate, 0.02% SDS, 2% blocking agent (manufactured by Roche), 50% formamide] heated to 50° C., was added 1 mg (final concentration 50 μg/ml) of Poly(U) (manufactured by Amersham Pharmacia Biotech) which had been quenched after heating at 95° C. for 5 minutes. This was sealed together with a membrane in hybridization bag, and pre-hybridization was performed at 50° C. for 2 hours. The hybridization buffer was transferred to a tube, and 5-6 μg of riboprobe (final concentration 0.25-0.3 μg/ml) which was quenched after heating for 5 minutes at 95° C. was added to the hybridization buffer and mixed. Then, the mixture was returned to a polyethylene bag, and the bag was sealed again. Hybridization was performed at 50° C. for 1 night to 3 days, while shaking in such way that the filter moved within the bag (approximately 12 rpm). Since 2 membranes spotted with the same DNA in (1) were prepared, 1 membrane was hybridized with a riboprobe of Thy-1 nephritis rat kidney and 1 membrane was hybridized with a riboprobe of control group rat kidney.

(4) Detection of Spots

The membranes were removed from the hybridization bags and washed with 2-fold SSC containing 0.1% SDS at 68° C. for 10 minutes. Then, the membranes were washed again in the same condition with using a fresh washing solution. Then, the washing at 68° C. for 15 minutes with 2-fold SSC containing 0.1% SDS was repeated two times.

After equilibrating the membranes by soaking them for 1 minutes in a small amount of buffer 1 [0.15 mol/l NaCl, 0.1 mol/l maleic acid, (pH 7.5)], the membranes were sealed in a polyethylene bag together with buffer 2 [buffer prepared by dissolving the blocking agent (manufactured by Roche) in buffer 1 at a final concentration of 1%] of an amount such that the membranes could move, and gently shaken at room temperature for 1 hour or more to perform blocking. After transferring buffer 2 in the polyethylene bags into a tube and adding alkaline phosphatase-labeled anti-DIG [antibody Anti-DIG-AP (manufactured by Roche)] in a volume of 1/10,000 and mixing, the mixture was returned to the bag and the bag was resealed. Then, the reaction was carried out while shaking gently at room temperature for 30 minutes to 1 hour. The membranes were removed from the bag, and was then subjected to 2 repetitions of washing while shaking for 15 minutes using buffer 1 added with 0.3% Tween 20. After equilibrating the membranes on opened bag by soaking them for 2 minutes in a small amount of buffer 3 [0.1 mol/l Tris (pH9.5), 0.1 mol/l NaCl, 50 mmol/l MgCl₂], CSPD which is a luminous alkaline phosphatase substrate (manufactured by Roche) which was diluted to 100 times with buffer 3 was applied on the membrane surface at 0.5-1.0 ml per 100 cm². After the membrane was covered with a bag, and the substrates were spread uniformly over the membrane surface, and reaction was allowed for 5 minutes. After excess moisture was removed, the bags were sealed, and reaction was allowed at 37° C. for 15 minutes. Then, X-ray film, Hyperfilm ECL (manufactured by Amersham Pharmacia Biotech), was exposed, and the film was developed. Exposure time was adjusted in such a way that the background concentration was the same level for those hybridized with riboprobe of Thy-1 nephritis rat kidney or riboprobe of control group rat kidney.

454 clones which had strongly hybridized with riboprobe of Thy-1 nephritis rat kidney in comparison with riboprobe of control group rat kidney were selected. Clones were identified from their array position, and each clone was cultured from the respective glycerol stock prepared in Example 3 (1), and plasmid DNA was prepared.

### EXAMPLE 4

### Nucleotide Sequence and Expression Analysis

(1) Nucleotide Sequence Determination

The nucleotide sequences of cDNA of the 454 clones selected by differential hybridization of Example 3 were determined by using a DNA sequencer, beginning with determining the nucleotide sequences from the ends. Regarding these nucleotide sequences, the homology to the sequences in nucleotide sequence databases GenBank, EMBL, and Gene Seq [manufactured by Derwent] was examined using the analysis program BLAST. As a results of this analysis, it was found that 148 clones among the 454 clones were cDNA of osteopontin, suggesting that osteopontin gene is expressed in a large amount in Thy-1 nephritis rat kidney. For clones which were considered to be novel nucleotide sequences from the result of homology analysis, the nucleotide sequence of the complete cDNA was determined. The thus obtained nucleotide sequences of the genes whose expression are increased in Thy-1 nephritis rat are shown in SEQ ID NOS: 1, 3, 5, 7, 9, 13, and 17-142. From the obtained nucleotide sequence of cDNA, the amino acid sequences of a polypeptide encoded by the gene were deduced, and with regard to these amino acid sequences also, the homology to the sequences in amino acid sequence databases SwissProt, PIR, GenPept, TREMBL and Gene Seq was examined using the analysis program BLAST.

(2) Expression Analysis

From the 454 clones selected in (1), clones of interest, mainly those of a novel nucleotide sequence, were selected, and the variations of the expression level in Thy-1 nephritis rat kidney of each gene was examined with time course by comparison with control group rat kidney by RT-PCR method. As a template, single strand cDNA was synthesized using SUPERSCRIPT Preamplification System for First Strand cDNA Synthesis Kit (manufactured by Life Technologies) in accordance with the kit's manual, from 5 μg each of the followings prepared in Example 1: total RNAs of kidney of rats on each of days 2, 4, 6, 8, 10, 13 and 16 after administration of anti-Thy-1 antibody OX-7; a mixture of equivalent amounts of the aforementioned total RNAs (called “Thy-1 mix”): and total RNA of kidney of rats of the control group administered with physiological saline. The single strand cDNA was finally dissolved in 250 μl of distilled water. As to the primers, based on the partial nucleotide sequence of cDNA of a clone to be examined, one set of forward primers and reverse primers for PCR having nucleotide sequences specific to that cDNA was designed and synthesized. (Primers of a chain length of 18-22 nucleotides were designed. The forward primer has the same nucleotide sequence as the partial nucleotide sequence of 5′ side of the cDNA, and the reverse primer has a complementary nucleotide sequence with the partial nucleotide sequence of 3′ side of the cDNA). The PCR conditions were as follows. For 1 μl of each single strand cDNA (equivalent to 20 ng of total RNA) to be employed as a template, there were added 2 μl of 10-fold reaction buffer (buffer attached to rTaq), 2 μl of 2.5 mmol/l dNTP, 1 μl of 10 μmol/l forward primer, 1 μl of 10 μmol/l reverse primer, 12.8 μl of distilled water, and Taq DNA polymerase rTaq (manufactured by Takara Shuzo). Then, the mixture was heated at 94° C. for 5 minutes, and subjected to 20 cycles of a reaction cycle of 1 minutes at 94° C. (denaturation)/1 minute at X° C. (annealing)/1 minute at 72° C. (elongation reaction) using an apparatus for PCR, and the mixture was then stored at 4° C. As the anneal temperature (X), an optimum temperature was selected depending on the primer. An aliquot of solution after reaction was subjected to electrophoresis, amplified DNA fragments were stained with fluorescent dye Cybergreen (manufactured by FMC BioProducts), and the amount of the amplified fragment was determined by Fluoroimager (manufactured by Molecular Dynamics). As a control, glycerardehyde-3-phosphate dehydrogenase (hereinafter referred to as “G3PDH”) gene, a housekeeping gene which is thought to show an almost fixed expression level in Thy-1 nephritis rat kidney of each stage and control group rat kidney, was subjected to RT-PCR (anneal temperature of 58° C.) in a similar manner with each template, using primers having the nucleotide sequences shown by SEQ ID NOS: 162 and 163, and the amount of the amplified fragments was determined. The comparison of control group rats with Thy-1 nephritis rats were then carried out based on the amount that was adjusted by dividing the amount of amplified fragments of each gene by the amount of amplified fragments of G3PDH gene for which amplification was performed with the same templates.

As a result, it was confirmed also by RT-PCR that 7 genes of TRDH-110, TRDH-122, TRDH-292, TRDH-344, TRDH-271, TRDH-284 and TRDH-363 show increased expression in Thy-1 nephritis rat kidney. These genes are described below.

(3) TRDH-271 gene

A nucleotide sequence of cDNA clone of TRDH-271 gene was determined (shown by SEQ ID NO:1), and its homology with sequences in databases was searched. As a result, there was no completely matching sequence and it was a novel nucleotide sequence. However, sequences showing extremely high homology existed in mouse EST (accession number AA981464 and the like).

In the nucleotide sequence of SEQ ID NO:1, ORF of 694 amino acids shown by SEQ ID NO:2 exists, and it is considered that TRDH-271 gene encodes a novel polypeptide having this amino acid sequence.

The result of RT-PCR (anneal temperature of 60° C.) using PCR primers having the nucleotide sequences shown by SEQ ID NOS: 145 and 146 showed that, over the entire period from day 2 to day 16 after administration of antibody, TRDH-271 gene showed a somewhat high expression level of 1.2 to 1.6 times more than the control group. RT-PCR for this gene was conducted with the number of reaction cycles being 23.

(4) TRDH-284 Gene

A nucleotide sequence of cDNA clone of TRDH-284 gene was determined (shown by SEQ ID NO:3), and its homology with sequences in databases was searched. As a result, there was no completely matching sequence and it was a novel nucleotide sequence. However, sequences showing extremely high homology existed in mouse EST (GenBank accession number AA050211 and the like).

In the nucleotide sequence of SEQ ID NO:3, ORF of 350 amino acids shown by SEQ ID NO:4 exists, and it is considered that TRDH-284 gene encodes a novel polypeptide having this amino acid sequence.

The result of RT-PCR (anneal temperature of 60° C.) using PCR primers having the nucleotide sequences shown by SEQ ID NOS: 147 and 148 showed that, over the period from day 4 to day 16 after administration of antibody, TRDH-284 gene showed a somewhat high expression level of 1.2 to 1.9 times more than the control group.

(5) TRDH-363 Gene

A nucleotide sequence of cDNA clone of TRDH-336 gene was determined (shown by SEQ ID NO:5), and its homology with sequences in databases was searched. As a result, there was no completely matching sequence and it was a novel nucleotide sequence. However, sequences showing extremely high homology existed in mouse EST and human EST (GenBank accession numbers AA117617, AA315924 and the like).

The nucleotide sequence of SEQ ID NO:5 encodes the amino acid sequence shown by SEQ ID NO:6, and it is considered that TRDH-336 encodes a novel polypeptide having this amino acid sequence.

The result of RT-PCR (anneal temperature of 64° C.) using PCR primers having the nucleotide sequences shown by SEQ ID NOS: 149 and 150 showed that, over the period from day 2 to day 16 after administration of antibody, TRDH-363 gene showed a high expression level of 1.5 to 2.8 times more than the control group.

(6) TRDH-292 Gene (Secreted Polypeptide Gene 2)

A nucleotide sequence of cDNA clone of TRDH-292 gene was determined (shown by SEQ ID NO:13), and its homology with sequences in databases was searched. The results showed that it had a high homology of 86% with deduced human secretory polypeptide gene 2 (WO98/39446; SEQ ID NO:15) having a homology with stromal cell derived factor-2. SEQ ID NO:16 shows an amino acid sequence encoded by human secretory polypeptide gene 2. Therefore, it was deduced that TRDH-292 gene was secretory polypeptide gene 2 in rat. SEQ ID NO:14 shows an amino acid sequence of a polypeptide of 220 amino acids encoded by the nucleotide sequence of SEQ ID NO:13.

The result of RT-PCR (anneal temperature of 60° C.) using PCR primers having the nucleotide sequences shown by SEQ ID NOS: 151 and 152 showed that, over the period from day 2 to day 16 after administration of OX-7, TRDH-292 gene showed a high expression level of 1.3 to 2 times more than the control group.

(7) TRDH-344 Gene (TSC-22 Analogous Protein-2)

A nucleotide sequence of cDNA clone of TRDH-344 gene was determined (shown by SEQ ID NO:7), and its homology with sequences in databases was searched. The results showed that it had a high homology of 78.8% with the gene of human TSC-22 analogous protein-2 (WO98/50425; SEQ ID NO:159). SEQ ID NO:160 shows the amino acid sequence of human TSC-22 analogous protein-2. Therefore it was deduced that TRDH-344 gene is TSC-22 analogous protein-2 gene in rat. SEQ ID NO:8 shows an amino acid sequence of rat TSC-22 analogous protein-2 of 150 amino acids encoded by the nucleotide sequence of SEQ ID NO:7.

The result of RT-PCR (anneal temperature of 60° C.) using PCR primers having the nucleotide sequences shown by SEQ ID NOS: 143 and 144 showed that, over the entire period from day 2 to day 16 after administration of antibody, TRDH-344 gene showed a somewhat high expression level of 1.2 to 1.6 times more than the control group.

(8) TRDH-122 Gene (mac25)

The complete nucleotide sequence of cDNA clone of TRDH-122 gene was determined (shown by SEQ ID NO:157), and its homology with sequences in databases was searched. The results showed that it had a high homology of 80% or more with genes reported as mouse mac25 [GenBank accession number AB012886; Cell Growth & Differ., 4, 715 (1993)] or human prostacyclin-stimulation factor [GenBank accession number S75725; Biochem. J., 303, 591 (1994); SEQ ID NO:11], and it was therefore deduced that TRDH-122 gene was mac25 gene of the rat. SEQ ID NO:12 shows an amino acid sequence of human prostacyclin-stimulation factor, i.e., human mac25. mac25 has also been reported as IGFBP-7 belonging to the IGF binding protein family [J. Biol. Chem., 271, 30322 (1996)], and is considered to be involved in the regulation of cell proliferation. mac25 has an activin binding ability and is reported to inhibit the proliferation of cancer cell-derived cell strains HeLa, P19 and Saos-2 at a concentration of 10⁻⁷ mol/L [Mol. Med., 6, 126 (2000)]. The nucleotide sequence of cDNA of TRDH-122 gene shown by SEQ ID NO:157 encoded the amino acid sequence shown by SEQ ID NO:158.

The result of RT-PCR (anneal temperature of 58° C.) using PCR primers having the nucleotide sequences shown by SEQ ID NOS: 153 and 154 showed that, over the entire period from day 2 to day 16 after administration of OX-7, TRDH-122 gene showed an increased expression level of approximately two times more than the control group.

(9) TRDH-110 Gene (α-2u Globulin)

A nucleotide sequence of cDNA of TRDH-110 gene was determined and its homology with sequences in databases was searched. The results showed that it matched with rat α-2u globulin cDNA (GenBank accession number U31287). Rat α-2u globulin is a secreted polypeptide having 162 amino acids (precursor containing a signal peptide has 181 amino acids), and has been reported to male-specifically form a vitreous body with a toxic substance in proximal renal tubule by administration of a certain species of nephrotoxic substance and cause cell proliferation or tumorigenesis [Crit. Rev. Toxicol., 26, 309 (1996)]. However, there have been no reports concerning the expression level of this gene or polypeptide in Thy-1 nephritis rat. The nucleotide sequence of rat α-2u globulin cDNA is shown in SEQ ID NO:9, and the amino acid sequence is shown in SEQ ID NO:10.

The result of RT-PCR (anneal temperature of 62° C.) using PCR primers having the nucleotide sequences shown by SEQ ID NOS: 155 and 156 showed that, while α-2u globulin gene showed an extremely high expression level of 44 times more than the control group on day 2 after administration of OX-7, the amount was 2.3 times on day 4 and the expression was hardly observed on day 6 and thereafter, thus showing a transient expression pattern. As the reaction solution for RT-PCR, solution obtained by adding 5% dimethylsulfoxide to the composition of (2) was used. Further, in the PCR, denaturation was carried out at 94° C. and not 95° C.

The changes with time course in the relative expression level of the above 7 genes in Thy-1 nephritis rat kidney are shown in Table 1, with the level of expression in control group rat kidney taken as 1.

TABLE 1
Changes with time course in the relative expression level of each gene in Thy-1 nephritis
rat kidney, with the expression level in control group rat kidney is taken as 1

| Gene | Overall* | Day 2 | Day 4 | Day 6 | Day 8 | Day 10 | Day 13 | Day 16 |
|---|---|---|---|---|---|---|---|---|
| TRDH-271 | 1.14 | 0.97 | 1.66 | 1.49 | 1.30 | 1.83 | 1.84 | 2.32 |
| TRDH-284 | 1.24 | 0.97 | 1.25 | 1.52 | 1.23 | 1.92 | 1.60 | 1.52 |
| TRDH-363 | 1.44 | 1.76 | 1.48 | 2.22 | 1.84 | 1.74 | 1.98 | 2.83 |
| TRDH-292 | 1.72 | 1.39 | 1.95 | 1.57 | 1.89 | 1.31 | 1.91 | 1.93 |
| TRDH-344 | 1.30 | 1.18 | 1.63 | 1.25 | 1.32 | 1.49 | 1.62 | 1.36 |
| TRDH-122 | 1.78 | 1.74 | 2.08 | 2.00 | 1.89 | 1.85 | 2.15 | 2.09 |
| TRDH-110 | 14.64 | 43.79 | 2.33 | 0.16 | 0.24 | 0.00 | 0.00 | −0.14 |

*“Overall” represents the values for a mixture of equal amounts of samples from each of days 2, 4, 6, 8, 10, 13 and 16.

### INDUSTRIAL APPLICABILITY

According to the present invention, a polypeptide useful in exploration and development of a therapeutic agent for actively repairing tissue which suffered from damage in renal disease, DNA which encodes the polypeptide, and an antibody which recognizes the polypeptide, as well as a method of utilizing the polypeptide, the DNA and the antibody, can be provided.

Sequence Listing Free Text

SEQ ID NO:143—Description of artificial sequence: Forward primer for amplification of TRDH-344 DNA.
SEQ ID NO:144—Description of artificial sequence: Reverse primer for amplification of TRDH-344 DNA.
SEQ ID NO:145—Description of artificial sequence: Forward primer for amplification of TRDH-271 DNA.
SEQ ID NO:146—Description of artificial sequence: Reverse primer for amplification of TRDH-271 DNA.
SEQ ID NO:147—Description of artificial sequence: Forward primer for amplification of TRDH-284 DNA.
SEQ ID NO:148—Description of artificial sequence: Reverse primer for amplification of TRDH-284 DNA.
SEQ ID NO:149—Description of artificial sequence: Forward primer for amplification of TRDH-363 DNA.
SEQ ID NO:150—Description of artificial sequence: Reverse primer for amplification of TRDH-363 DNA.
SEQ ID NO:151—Description of artificial sequence: Forward primer for amplification of TRDH-292 DNA.
SEQ ID NO:152—Description of artificial sequence: Reverse primer for amplification of TRDH-292 DNA.
SEQ ID NO:153—Description of artificial sequence: Forward primer for amplification of TRDH-122 DNA.
SEQ ID NO:154—Description of artificial sequence: Reverse primer for amplification of TRDH-122 DNA.
SEQ ID NO:155—Description of artificial sequence: Forward primer for amplification of TRDH-110 DNA.
SEQ ID NO:156—Description of artificial sequence: Reverse primer for amplification of TRDH-110 DNA.
SEQ ID NO:161—Description of artificial sequence: Primer having T7 promoter and polythymidylic acid sequence.
SEQ ID NO:162—Description of artificial sequence: Forward primer for amplification of G3PDH DNA.
SEQ ID NO:163—Description of artificial sequence: Reverse primer for amplification of G3PDH DNA.

1-44. (canceled)

45. A method for treating renal disease, which comprises administering a polypeptide comprising an amino acid sequence selected from the group consisting of SEQ ID NOs: 8, 10, 12, 14, 16, 158 and 160 to a patient in need thereof.

46-50. (canceled)

51. The method according to claim 45, wherein the polypeptide comprises SEQ ID NO:8.

52. The method according to claim 45, wherein the polypeptide comprises SEQ ID NO:10.

53. The method according to claim 45, wherein the polypeptide comprises SEQ ID NO:12.

54. The method according to claim 45, wherein the polypeptide comprises SEQ ID NO:14.

55. The method according to claim 45, wherein the polypeptide comprises SEQ ID NO:16.

56. The method according to claim 45, wherein the polypeptide comprises SEQ ID NO:158.

57. The method according to claim 45, wherein the polypeptide comprises SEQ ID NO:160.

58. The method according to claim 45, wherein the polypeptide consists of SEQ ID NO:8.

59. The method according to claim 45, wherein the polypeptide consists of SEQ ID NO:10.

60. The method according to claim 45, wherein the polypeptide consists of SEQ ID NO:12.

61. The method according to claim 45, wherein the polypeptide consists of SEQ ID NO:14.

62. The method according to claim 45, wherein the polypeptide consists of SEQ ID NO:16.

63. The method according to claim 45, wherein the polypeptide consists of SEQ ID NO:158.

64. The method according to claim 45, wherein the polypeptide consists of SEQ ID NO:160.

1-44. (canceled)

45. A method for treating renal disease, which comprises administering a polypeptide comprising an amino acid sequence selected from the group consisting of SEQ ID NOs: 8, 10, 12, 14, 16, 158 and 160 to a patient in need thereof.

46-50. (canceled)

51. The method according to claim 45, wherein the polypeptide comprises SEQ ID NO:8.

52. The method according to claim 45, wherein the polypeptide comprises SEQ ID NO:10.

53. The method according to claim 45, wherein the polypeptide comprises SEQ ID NO:12.

54. The method according to claim 45, wherein the polypeptide comprises SEQ ID NO:14.

55. The method according to claim 45, wherein the polypeptide comprises SEQ ID NO:16.

56. The method according to claim 45, wherein the polypeptide comprises SEQ ID NO:158.

57. The method according to claim 45, wherein the polypeptide comprises SEQ ID NO:160.

58. The method according to claim 45, wherein the polypeptide consists of SEQ ID NO:8.

59. The method according to claim 45, wherein the polypeptide consists of SEQ ID NO:10.

60. The method according to claim 45, wherein the polypeptide consists of SEQ ID NO:12.

61. The method according to claim 45, wherein the polypeptide consists of SEQ ID NO:14.

62. The method according to claim 45, wherein the polypeptide consists of SEQ ID NO:16.

63. The method according to claim 45, wherein the polypeptide consists of SEQ ID NO:158.

64. The method according to claim 45, wherein the polypeptide consists of SEQ ID NO:160.


SEQUENCE LISTING (163 sequences)

SEQ ID NO: 1 - DNA, 2470 bp, Rattus norvegicus
  CDS (79)..(2160)
ggcggggatctgcgcggcggcggaggcgggacctctggcatcagtagcaccgtgagcccagacattcctctctgagtcatgacggatgccaagtatgtcctctgccgatgggagaagcgactgtggcctgcaaaggttttggccagaactgagacttcagcaaaaaacaagagaaaaaaggaattctttctagatgttcaaatactctcactaaaggaaaagatccaggttaagagctcagccgtggaggcactgcagaagtcacacattgagaacattgccgccttcttggcctctcagaatgaagtcccagctactcctctggaggagctgacttaccgacggtccctgcgagtggccctggatgtcttgaacgagaggaccagtttgagtcctgaaagtcatccagtcgaaaatgggagcacaccatctcagaagggcaagccagatgcagatgtggcctcgcgggtctctagtgctccttctccatcttttctcagtgaagatgatcaggctgtggcagcccagtgtgcatccaagaggaggtgggagtgcagtccaaaaagcctgtcgccgttgtctgcctcggaagaggatctcaggtgcaaagtggaccccaagacaggcctctcagagagtggagccctggggactgaagtgcctgcccccactggggatgagtctcagaatggctctgggtcacagctggaccatggacaggagagcacaaccaaaaagagacagaggaattcgggagagaaacctgcccggcgtggaaaagcagagtctggcctttccaagggagacagtgtcgcagagagcggaggacaggcaagcagctgtgtggccctggcttcacccaggctgccctcccaaacctgggagggggatccatgtgctggagtcgaaggctgtgacccagttgagtcatctggcaacatcaggccgcttctggactctgagagaagcaaaggacgcctcacaaagaggccacgcttggacggaggccggaacccactgcccagacatctaggaaccagaactgtgggggcagtgccctcccgtaggagctgctctggggaggtcacgacgctgcgcagggctggagacagtgacagaccagaggaagatcctatgtcttcagaagaatctacagggttcaagtccgtccactccctgctggaggaggaggaggaggaggaggaagaggaggaggaaccaccccggatccttctgtatcacgaaccacgatcatttgaagtaggaatgctggtctggcttaaataccaaaaatacccattctggccagccgtggtcaagagtgtccggcggagggacaagaaggccagtgtgctcttcattgagggcaacatgaatcccaagggccgaggaatcaccgtgtcgctgcgacggctcaagcactttgactgcaaggaaaagcatgcactactggacagagccaaagaggactttgcccaggctattggctggtgtgtctcgcttatcactgactaccgcgtgcggctgggctgcggctccttcgccgggtcgttcttggaatattacgctgctgatatcagctatcctgtgcgcaagtctatccaacaggacgtcctggggaccaggtttcctcagctgggcaagggggaccctgaggagcctatgggggacagccggctgggacagtggcggccatgcaggaaggtgctgcctgaccgctccagggctgcccgggataaagccaaccagaagctggtggagtacatcgtgaaggccaagggtgcagagagccacctgcgggctatcctgcacagccgcaagccctcacgctggctgaagacgttcctgagctccaatcagtacgtgacatgcatggagacgtacctggaggatgaggcgcagctggatgaggtggtggagtacctgcagggcgtctgccgagacatggatggcgagatgcctgcgcgcggcagcggcgaccgcatccgtttcatcctggatgtgctgctgcctgaggcgatcatctgcgccatctcggcagtggaggcagtggactacaagacagccgagcagaagtacctccgtggccccacactcagctaccgggaaaaggaaatctttgacaatgaactcctggaggagaggaaccgtcgccgtcgctgatgccgtagtctccacctggccagcaccgtgtctgtggcctgccagaggcctgtgagaatgtgctagaagcaagaggcctagtaatgtgctgactttgatctgtgcatgggttctgcgtcttcagccctgagcctgggagatcagaggccatcttcacactagaagactgctgcatctatgaacagctgcttctggaagtttctgtgtgtgtacgcgtgtatgtttggttttatttttttaattattattttgtttataaatgcgtttgaatgcaaaaaaaaaaaaaaaaaaaaaaaaaaaaaaaaaa

SEQ ID NO: 2 - protein, 694 aa, Rattus norvegicus
MTDAKYVLCRWEKRLWPAKVLARTETSAKNKRKKEFFLDVQILSLKEKIQVKSSAVEALQKSHIENIAAFLASQNEVPATPLEELTYRRSLRVALDVLNERTSLSPESHPVENGSTPSQKGKPDADVASRVSSAPSPSFLSEDDQAVAAQCASKRRWECSPKSLSPLSASEEDLRCKVDPKTGLSESGALGTEVPAPTGDESQNGSGSQLDHGQESTTKKRQRNSGEKPARRGKAESGLSKGDSVAESGGQASSCVALASPRLPSQTWEGDPCAGVEGCDPVESSGNIRPLLDSERSKGRLTKRPRLDGGRNPLPRHLGTRTVGAVPSRRSCSGEVTTLRRAGDSDRPEEDPMSSEESTGFKSVHSLLEEEEEEEEEEEEPPRILLYHEPRSFEVGMLVWLKYQKYPFWPAVVKSVRRRDKKASVLFIEGNMNPKGRGITVSLRRLKHFDCKEKHALLDRAKEDFAQAIGWCVSLITDYRVRLGCGSFAGSFLEYYAADISYPVRKSIQQDVLGTRFPQLGKGDPEEPMGDSRLGQWRPCRKVLPDRSRAARDKANQKLVEYIVKAKGAESHLRAILHSRKPSRWLKTFLSSNQYVTCMETYLEDEAQLDEVVEYLQGVCRDMDGEMPARGSGDRIRFILDVLLPEAIICAISAVEAVDYKTAEQKYLRGPTLSYREKEIFDNELLEERNRRRR

SEQ ID NO: 3 - DNA, 1585 bp, Rattus norvegicus
  CDS (48)..(1097)
accggaagttgtatcgaggcttccgcacatggatacttctggagaacatgccactggtcgtggtttgcgggctgccgtccagcggcaagagccggcgtacggaagagttacgtcgggcgctgaccggcgagggacgttcggtgtatgtggtggacgatgcttcggtgctgggcgcgcaggattccactgtgtacggcgactctgcgggtgagaaggcgctacgtgctgcgctgcgggccgcggtagagcggcgcctgagccggcaggacgtggtcatcctagactccatgaactacatcaaggggttccgctacgagttgtactgccttgcgcgagctgtgcgcacgccgctctgcttagtttactgcataaggcccggctggccaagccgcgggcttccggtgcctggcgcctgcgagagctcggacccggctgtcagtgtgagctggaggccgcgcgccgactacggcgagaagactcaggcggtcggcgctgtagagcagcgcgccatcagccccttagcaaatgggggagtcccgaccgctgtccccaaggaactggatccaaaggatatcctgccatcaaatcctccagctgtaatgactccggaatccgagaaatctgcagagcctgcgccatgtgcctttcctcccgaacttttggagtccttagcgctgcgctttgaagctcccgactctcggaaccgctgggatcgacccttgttcaccgtggtgggtttagaagagccattgcccctggctgagatccggtctgcactgttcgagaatcgggctcccccaccccatcagtctacacagtcccagcccctggcctctggcagctttctacaccagttggatcaggccacgagccaggtgttgactgctgtgatggaaacacagaagagcgctgtacccggagacttactaacgcttcctggcaccacggagcacctccgatttacccgtcccttgaccttggcagaattgagtcgcctccgtcgccagtttatttcctacactaaaatgcatcccaacaatgagaacctgcctcaattggccaacatgtttcttcagtatctgaaccagagtttgcactaatgggatagtggtctgcagcggtggctcttgtctgaattcccctgtactttggctaggaaaaatagtccgaaggtctgcaaagcgcactgtagtactgagatgctaaatttgactcattttcttaactgcctctgccataccctgagtgtgctgcataagctgaggcattgagcaccagctccaaaaataccaggtggcttcggttggaatctacttggggattcttcatacactgttttcctttcatcgggacggagaattgttaagtcaactgtgagtagaaaccgaagataacagttttgtatttatgatggccctttcatactacaaatacttttgagcacagtgcctcttgctatctatcctggaacttcgaacacagataaatcttgttctgcccctgggaaactgatatttgtataagacagccattagatatttcctctaataaaatcttctaaaattaaaaaaaaaaaaaaaaaaaa

SEQ ID NO: 4 - protein, 350 aa, Rattus norvegicus
MPLVVVCGLPSSGKSRRTEELRRALTGEGRSVYVVDDASVLGAQDSTVYGDSAGEKALRAALRAAVERRLSRQDVVILDSMNYIKGFRYELYCLARAVRTPLCLVYCIRPGWPSRGLPVPGACESSDPAVSVSWRPRADYGEKTQAVGAVEQRAISPLANGGVPTAVPKELDPKDILPSNPPAVMTPESEKSAEPAPCAFPPELLESLALRFEAPDSRNRWDRPLFTVVGLEEPLPLAEIRSALFENRAPPPHQSTQSQPLASGSFLHQLDQATSQVLTAVMETQKSAVPGDLLTLPGTTEHLRFTRPLTLAELSRLRRQFISYTKMHPNNENLPQLANMFLQYLNQSLH

SEQ ID NO: 5 - DNA, 1879 bp, Rattus norvegicus
  CDS (343)..(1410)
ctagcccgggcaggcccggcggggggggcgttgaccttgcgggcggtcaaaccggccacccgtttttccctggcggtggcgctcgggagtctgggtgggggcctcggagccaggggccacggactgcatcacggtagagagattcgcgagcctcaggcgagggacgcaacctccagctccgcggagaccgagggtggccacgtccagggacatctccggttcattcattgggttcctactgtgtgctcttatacggcgctcagccagcccaactgatgtggagcgctgtgcgcggccctgctaggcttctttgtggatggccggggcgaggtcctcttcactatggcccggcgtgcacggagtagcagggcatggcactttgtcctgagtgcagcacgccgagatacagatgctcgagctgtggctctggcaggcaactctaactggggctacgactctgatgggcagcacagcgactccgactctgaccctgagtactcttccctgccaccatccatccccagtgctgtgcctgtgacaggagagtccttctgtgactgtgagggccagaatgaggctaccttctgcaacagtttacacacagcacaccgtggcaaggactgccgttgtggtgaggaggatgaggattttgattgggtatgggatgacctgaacaagtcctcagccaccttgctgagctgtgataatcgaaaggttagctttcacatggagtacagctgtggcacagcagccattcggggcaccaaggagctaggggatggccaacacttctgggaaatcaagatgacctctccggtgtatggcactgatatgatggtgggcatcgggacatcagacgtagacctggacaagtaccaccacacgttctgcagcctgctgggcagggatgaagacagctgggggctctcctacacggggctcctccaccacaaaggcgacaagacgagcttctcttcacgcttcggccagggctctatcattggcgtacacttggacacctggcatgggacactgacttttttcaagaataggaagtgcataggagtggctgccactcggcttcagaacagaaggttctacccgatggtctgctcgaccgccgccaagagcagcatgaaggtcattcgctcctgtgccagctccacatccctgcagtacctgtgctgctaccgcctgcgccagttgcggccagactcaggggacaccctcgagggcctgcccttgccacccggcctcaagcaggtgctgcataacaagctgggctgggtcctgagcatgaactgcagccactggacatcccctgcaccccctccgggcacagctgccccagccgctgagagagattcccgggagaccaggccctgtcagaggaagcgctgccgaagaagctgacttctccccgggaatgcagacacctttctttcttgcccttccagggcagcaggagaggggagaacggaggtctaggcttttccctgtctccccgaggccaggacagtcttctctgttggccatggagtgtgacagctgttctaccgcctgtgctggtagggaaacagcactccttcctgtttgtcctttgagttgccatgtatcctgggagctgcagccaggcgtctggacctagattccaagcctgggaggctggctgacgaagtggagtgcattcatatcccagggaagagatgggctgtcccgacccacaggtctgtggggttttcctgacttgcattgcatgttgtcagcgcctgctcctgtcacagagatgtcagtgggtgccctgggaagggattctgtctcgtccccataggttctatcattaaaagcgtcctcacaaatgaaaaaaaaaaaaaaaaaa

SEQ ID NO: 6 - protein, 356 aa, Rattus norvegicus
MARRARSSRAWHFVLSAARRDTDARAVALAGNSNWGYDSDGQHSDSDSDPEYSSLPPSIPSAVPVTGESFCDCEGQNEATFCNSLHTAHRGKDCRCGEEDEDFDWVWDDLNKSSATLLSCDNRKVSFHMEYSCGTAAIRGTKELGDGQHFWEIKMTSPVYGTDMMVGIGTSDVDLDKYHHTFCSLLGRDEDSWGLSYTGLLHHKGDKTSFSSRFGQGSIIGVHLDTWHGTLTFFKNRKCIGVAATRLQNRRFYPMVCSTAAKSSMKVIRSCASSTSLQYLCCYRLRQLRPDSGDTLEGLPLPPGLKQVLHNKLGWVLSMNCSHWTSPAPPPGTAAPAAERDSRETRPCQRKRCRRS

SEQ ID NO: 7 - DNA, 1055 bp, Rattus norvegicus
  CDS (102)..(551)
ctcaaaccctgccctccctgagggtagaagtggagtctgggggtttagcagccggaacccctcctctgtcacggagaagagatggagcagttcggctgaggatggagttagttgctccagaggagacagggaaggtacctcccatcgactctcgccccaactccccagccctctacttcgatgccagcctggttcacaagtctccagacccattcggagctgcagcagcccagagcctcagcctggctcggtccatgttggccatcagcggtcacctggacagtgatgacgacagtggttccggaagcctggttggcattgacaacaagattgaacaagccatggacttggtgaagtcccacctcatgtttgccgtgcgagaggaggtggaggtgctgaaggagcagatccgggacctggcagagcggaatgctgcactggagcaggaaaatggattgctgcgtgccctggccagcccggagcagctggcccagctgccatcctcggggctcccaaggctcgggccctctgcacccaatgggccttccatctgagccttctttccctcacaatgtgcctttgggggctgccactggccgccgggccttgtgccagcagcctgccccctcttcctatgtagctttaatgcccacgcccgaccccaatgcccagggatgggagttgaggctaaatattggcctgtcccttcccacctggtctccccagaagcctcaggccttgccggaagagaaagaacccaggaggggatgtttatctgaagcccctcatccatgaaagaacccagccccacctccttccctgggtattagtgttctggggagcccctcagcagcagatggctcagaaagatttggaggttccctggcaggccccctcaccatcccaccttgttctcttcaagtgccccctctcctctgcccagggagggggtatggacagtatcttcaacttcttggattcaggttgttattaaaataataattataattaaaaaaaatctgaagaaacttgaaaaaaaaaaaaaaaaaaaaaaa

SEQ ID NO: 8 - protein, 150 aa, Rattus norvegicus
MELVAPEETGKVPPIDSRPNSPALYFDASLVHKSPDPFGAAAAQSLSLARSMLAISGHLDSDDDSGSGSLVGIDNKIEQAMDLVKSHLMFAVREEVEVLKEQIRDLAERNAALEQENGLLRALASPEQLAQLPSSGLPRLGPSAPNGPSI

SEQ ID NO: 9 - DNA, 878 bp, Rattus norvegicus
  CDS (54)..(596)
ggcacgagcagagagattgtcccaacagagaggcaattctattccctaccaacatgaagctgttgctgctgctgctgtgtctgggcctgacactggtctgtggccatgcagaagaagctagttccacaagagggaacctcgatgtggctaagctcaatggggattggttttctattgtcgtggcctctaacaaaagagaaaagatagaagagaatggcagcatgagagtttttatgcagcacatcgatgtcttggagaattccttaggcttcaagttccgtattaaggaaaatggagagtgcagggaactatatttggttgcctacaaaacgccagaggatggcgaatattttgttgagtatgacggagggaatacatttactatacttaagacagactatgacagatatgtcatgtttcatctcattaatttcaagaacggggaaaccttccagctgatggtgctctacggcagaacaaaggatctgagttcagacatcaaggaaaagtttgcaaaactatgtgaggcgcatggaatcactagggacaatatcattgatctaaccaagactgatcgctgtctccaggcccgaggatgaagaaaggcctgagcctccagtgctgagtggagacttctcaccaggactctagcatcaccatttcctgtccatggagcatcctgagacaaattctgcgatctgatttccatcctctgtcacagaaaagtgcaatcctggtctctccagcatcttccctaggttacccaggacaacacatcgagaattaaaagctttcttaaatttctcttggccccacccatgatcattccgcacaaatatcttgctcttgcagttcaataaatgattacccttgcactt

SEQ ID NO: 10 - protein, 181 aa, Rattus norvegicus
MKLLLLLLCLGLTLVCGHAEEASSTRGNLDVAKLNGDWFSIVVASNKREKIEENGSMRVFMQHIDVLENSLGFKFRIKENGECRELYLVAYKTPEDGEYFVEYDGGNTFTILKTDYDRYVMFHLINFKNGETFQLMVLYGRTKDLSSDIKEKFAKLCEAHGITRDNIIDLTKTDRCLQARG

SEQ ID NO: 11 - DNA, 1124 bp, Homo sapiens
  CDS (23)..(868)
gccgctgccaccgcaccccgccatggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataaacctccagaatattattagtctgcatggttaaaagtagtcatggataactacattacctgttcttgcctaataagtttcttttaatccaatccactaacactttagttatattcactggttttacacagagaaatacaaaataaagatcacacatcaagactatctacaaaaatttattatatatttacagaagaaaagcatgcatatcattaaacaaataaaatactttttatcacaaaaaaaaaaaaaaaa

SEQ ID NO: 12 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 13 - DNA, 1043 bp, Rattus norvegicus
  CDS (15)..(674)
ggccggccggcacgatgttgggcgcgagccgcgggttagcgggtctgacgctgctggggctgctgctggcgctctcggtgcggagcggtggcgcgtcgaaggccagcgccgggctagtgacctgcgggtcagtgctgaagctactcaacacccaccacagagtgcggctgcactcacatgacatcaaatacggatccggcagcggccaacagtcggtaaccggcgtggaggcgtccgacgatgccaatagttactggcgaattcgcggcggctccgagggtgggtgcccgcgcgggctcccagtgcgctgtgggcaggcagtgcggctcacgcacgtgctcaccggcaagaacctgcacacgcaccacttcccgtcaccgctatccaacaaccaggaggtgagtgcttttggggaagacggtgagggtgatgacctggacctgtggacagtacgatgttctgggcaacactgggagcgagaggccagtgtccgtttccagcatgttggcacctctgtgttcctgtcagttactggtgaacagtatggtaacccaatccgtgggcagcatgaggtgcatggcatgcctagtgccaatgcacacaacacgtggaaggccatggaaggaatcttcatcaagcccggagcagatccctccacaggtcacgatgaactctgagccggatgggaagggagggtggctgagtgggaatccgcagggctgctcttgtgtaagactctgtaggggccctcaagtgcctttctgattaaagaatgttggtttgtgattatttttgctgtaccctggggaggacctgagggtgctagtcatatctgtccacatcatcatctcacatgtctcaagtacctgttcaaataatttttgagaccgtcccactatgtatccctggctggcctggaactcccagagatccacttgcctctgcctcctgagcgctggtattaaaggtgtatacgaccacagctggccccaacctgttcaataaactaatttttattacagtgtgaaaaaaaaaaaaaaaaa

SEQ ID NO: 14 - protein, 220 aa, Rattus norvegicus
MLGASRGLAGLTLLGLLLALSVRSGGASKASAGLVTCGSVLKLLNTHHRVRLHSHDIKYGSGSGQQSVTGVEASDDANSYWRIRGGSEGGCPRGLPVRCGQAVRLTHVLTGKNLHTHHFPSPLSNNQEVSAFGEDGEGDDLDLWTVRCSGQHWEREASVRFQHVGTSVFLSVTGEQYGNPIRGQHEVHGMPSANAHNTWKAMEGIFIKPGADPSTGHDEL

SEQ ID NO: 15 - DNA, 844 bp, Homo sapiens
  CDS (39)..(701)
ggcccctgggcccgaggggctggagccgggccggggcgatgtggagcgcgggccgcggcggggctgcctggccggtgctgttggggctgctgctggcgctgttagtgccgggcggtggtgccgccaagaccggtgcggagctcgtgacctgcgggtcggtgctgaagctgctcaatacgcaccaccgcgtgcggctgcactcgcacgacatcaaatacggatccggcagcggccagcaatcggtgaccggcgtagaggcgtcggacgacgccaatagctactggcggatccgcggcggctcggagggcgggtgccgccgcgggtccccggtgcgctgcgggcaggcggtgaggctcacgcatgtgcttacgggcaagaacctgcacacgcaccacttcccgtcgccgctgtccaacaaccaggaggtgagtgcctttggggaagacggcgagggcgacgacctggacctatggacagtgcgctgctctggacagcactgggagcgtgaggctgctgtgcgcttccagcatgtgggcacctctgtgttcctgtcagtcacgggtgagcagtatggaagccccatccgtgggcagcatgaggtccacggcatgcccagtgccaacacgcacaatacgtggaaggccatggaaggcatcttcatcaagcctagtgtggagccctctgcaggtcacgatgaactctgagtgtgtggatggatgggtggatggagggtggcaggtggggcgtctgcagggccactcttggcagagactttgggtttgtaggggtcctcaagtgcctttgtgattaaagaatgttggtctatgaaaaaaaaaaaaaaaaa

SEQ ID NO: 16 - protein, 221 aa, Homo sapiens
MWSAGRGGAAWPVLLGLLLALLVPGGGAAKTGAELVTCGSVLKLLNTHHRVRLHSHDIKYGSGSGQQSVTGVEASDDANSYWRIRGGSEGGCRRGSPVRCGQAVRLTHVLTGKNLHTHHFPSPLSNNQEVSAFGEDGEGDDLDLWTVRCSGQHWEREAAVRFQHVGTSVFLSVTGEQYGSPIRGQHEVHGMPSANTHNTWKAMEGIFIKPSVEPSAGHDEL

SEQ ID NO: 17 - DNA, 927 bp, Rattus norvegicus
  n all: unknown
angctcgaaattaaccctcactaaagggaacaaaagctggagctccaccgcggtggcggccgctctagaactagtggatcccccgggctgcaggaattcggcacgagggacagagagcgcatggagatgggaagactgtcgccacccagacagccacgcaagtgcctttaactttgagaaggccttttctccttttctgatttggtgctacggactcacgacagaactcagacaccagcagacaagagtctcggcctaggtggcggtggccactctggccagacgaaagccagtttgtttctgatttttgccttctttacaactaagcagttttgtgtagcagggcaggcctgttccggccagctttcttttaagatccgggttaattttcctttccagcagccttctctctggagtggcctctaccacactaacaggaggtgtcttcagagtatggacagctagccacgaggcccctccgctcctgggagggctactccgttcctaggacaccagaggccacaaactagggttgggccacaagcacacaatgctttcttccacggcaggaattcataccaaaaccacaagcaaaaaacaaaacaaaaaaaaaaaaaaaaaactcgagggggggcccggtacccaattcgccctatagtggagtcgtattacaattcantgggccgtcgttttacaacgtcgtgactgggaaaaccctggggttacccaactttaatcgccttgnagcaaatcccccttttggccagctggggtaatagcgaagaaggcccgnaccggattggcctttccaaaagttgcgcagcttgaatggggaatgggaaattgtaaggggtaanaatttggttaaaaatcggngttaaaattttggtaaaatcaggcccanttttttaacccaaaaaggggggg

SEQ ID NO: 18 - DNA, 933 bp, Rattus norvegicus
  n all: unknown
caaggtcgaaattaaccctcactaaagggaacaaaagctggagctccaccgcggtggcggccgctctagaactagtggatcccccgggctgcaggtttttttttttttttttcacnagcttgatctatctctccgttctccatggctctgatgtcaggccgcagcctgcagtcagtcttgggaatcacggtctccatctccttgtctacctcattcaagaccatggcaaagctggtaaaattatacatctgagcagagttgggaggccgcggggctatccgccacaggagcgcactgccaggaataacaaatacgctctcagagtccggcactgggcatttcgtcagactcctcagatggcgcttgcctgtttggctgttcttcttctcttctgtgtttttcttatcgttttttttgtaagcgtcaaaagtggcagggtctacactgtacaagcactcggtccacttcccgtagagagcacagagtttctttttgcttttancttggatgtagccttcaactttgtgtaattccttgccaaaaagaccacatggcttaaaattcaacacacacttgtccccagtcttgtggtttangatttccacattgccatactgttcgatccagagtttgcccacgatgatgttatgcacacagcagtggggtttgtccatgtgtatggggggggcccggtacccaatcgncctanagtgagtcgtaatacaattcacngggccggtcggtttacaaagtcgtggaatgggaaaaacctgggcggttacccaaacttnaatcgccttgcagcaaaaaccccctttcggcaaanngggggnaaaaagcgaanaaggcccggaancggaattggcnccttccccaaaaagnttgcggacnctngaaaagggggaaatgggcaaaatggaaaancggttaaaa

SEQ ID NO: 19 - DNA, 933 bp, Rattus norvegicus; identical to SEQ ID NO: 18
  n all: unknown

SEQ ID NO: 20 - DNA, 942 bp, Rattus norvegicus
  n all: unknown
caagngcgaaattaaccctcactaaagggaacaaaagctggagctccaccgcggtggcggccgctctagaactagtggatcccccgggctgcaggctgactttataggaaaactgattatatcaatgtgtatatgtgttatatatacatatattcaatactgccttctctttttgtctacagtatcaaaattgactgacggaatcatgaaaagaatgttccccatcaccatttagagttttatttttgttttctttgtttatcaatgaatggtgtaagaatcaagtctcttgtttttttgaagaaaaaaagcaatattccttgaagagcaaggaggattgaaggattttgtttgagtgaggaacagagttcataactagtttgttggatacttgtaaggttggtatctttgtgggcctatatactctaaaatgaaccttggtggcttgtgggccattacttgacctatgaatctttaagggcacaatcagttatcttttacatataaagatcgcttggagtgatggccaccgctcctgcccgncctccctccctcccttccttccgggaaaannngcgggncnnnnnnccnccnnncnnncccnncnnnnnnnnnnnnnnnnnnnnngccgngggggggnccggnnnccnnnngnccnnnnnnggggncgnnnnnnnnnnnnnnngggnngnngnnnnnnnnngnnnnnnnnnggggnnnanccnggngnnnnnncnnnnnnnnnnngnnnngnnnnnnnnnnncccnnnnnnnnnnnngggggnnnnnnnnnnnnggnnnngnnnnngnnnngnnnnnnnnnnnnnnnnnnnnnnnnnnnnnnnggnnnnnngggnnnnnnnnnnnnnngnnnnnnnnnnnnngnnnnnnnnnnngnggnnnnnnnnnnnnnnnnnnnnnnnnnnnnnnnnnnnnnnnnnn

SEQ ID NO: 21 - DNA, 929 bp, Rattus norvegicus
  n all: unknown
ncaagcgcngaaattaaccctcactaaagggaacaaaagctggagctccaccgcggtggcggccgctctagaactagtggatcccccgggctgcagggttgcgggctttgaaccgcctggccgcgcggcccgggggccagcccccaaccctgctccttctgcccgtgcgcggccgcaagacccgccacgatccgcctgccaagtccaaggtcgggcgcgtgaaaatgcctcctgcagtggaccctgcggaattgttcgtgttgaccgagcgctaccgacagtaccgggagacggtgcgcgctctcaggcgagagttcacattggaggtgcgagggaaattgcacgaggcccgagccggggttctggctgagcgcaaggcgcaagaggccatcagagagcaccaggagctgatggcctggaaccgggaggagaaccggagactgcaggaactacgggatagctaggttgcagctcgaagcacaggcccaggagctgcggcaggctgaggtccaggcccagagggcccaggaggagcaggcttgggtgcaactgaaagaacaagaagttctcaaactgcaggaggaggccaaaaacttcatcactcggggagaacctggagggcacggatagaagaggccttggactctccgaagagttataactggggcggttcaccaaagaagggcaggtggttcaggaactgagaacagaggctcttcaggcccaaataaggacatgcttgcctaaggatggatattggggtagaaattggtgcatcccaggagggtngcaanancttgttccagagcnagcccccatttcatttctnaganttngcaccaaggtatagtaccctgttcttgacaccaacatnccaaacttcgggacagcanttaaaactcctgggnaanttctatcaaaccagaagg

SEQ ID NO: 22 - DNA, 925 bp, Rattus norvegicus
  n all: unknown
ncaagcgncgaaattaaccctcactaaagggaacaaaagctggagctccaccgcggtggcggccgctctagaactagtggatcccccgggctgcaggaattcggcacgagatggcagacgtcctcccaggtatgcagaagacaacttctggagtcagttctgtcaaccatgtgggtctcagggatggaactcagttggtcatgattggcagcaagcacctttacctgctgacccacctcagcactcctgatggagcagatgtagatgaacaatatcatctatgaaacattccaaacaaaactaacttgaatccatcaagccttcccatcaaacacgcaatttttttaatttgttttattattttttgatgtctgtgtgttatgtctgcatgtatgtctgtgtgccatgtttgttcccggtgccccctggaagtcagaagaaggaatcagatcccctagaactggagtttcagaaagatgagctgcaggtgggggctgggaattgaacctgtgtcctctggaagagcagctggtgctcttaacagctgaacacctctccagtgccaaacacaccatttataagaaatacaacaggtggaaataacaaattctgtggccattctggaggataactggtgtataagcttcaacaatgtatcatcctggaagaaacaatggctgtgtggggaaaaaaaaaatctaagggacattacagcctgacccagatccnattctggaacagacaagctataaaacacctttcagcacaattggaaggaggaacgaaagccatgggaatatttggataagatgaagttgtgttgccatgcaagccttgggaggncattaaggaaacgggcaagtccncaaaaagggggngntgnnccanaanaaccccggggttaaaaannnnaaaaggggggggg

SEQ ID NO: 23 - DNA, 1828 bp, Rattus norvegicus
  n all: unknown
ncaagcgcgaaattaacnntcactaaagggaacaaaagctggagctccaccgcggtggcggccgctctagaactagtggatcccccgggctgcagggttttgccgaggggtcttgggctggggcggacagtgtacgggatggaggcgactttggagcagcatttggaggacacaatgaagaatccatccattgttggagtcctatgcacagattcacaaggacttaatctgggctgccgtggtaccctgtcggatgagcatgctggagtgatatctgttctcgcccagcaggcagctaagctgacctctgaccccaccgacatccctgtagtgtgtttagagtcagataacgggaatgttatgatccagaaacacgatggcatcacagtggctgtgcacaaaatggcctcttgacatctgatgccagctctccagtggtctcccaccgggattcagtcatgcctgtctcagttaacttgtaaaactattaaagttccagaaatcgggccattcacttaatgtccaatgtggacttcttattaatatgacagtcagttaccaagacgtcagttaggagtgtggtggccttgtctgggcttttgtcactctgctctttggtgacagccactgtagtccaggatcatatccctcaggcctagaactgtgtagcccaggctgacttcaaatttatggtcttcctgcttcaaactcctatatcctggggatttagcattgtcatgggtctaggtcactttgtatatagaactttgttgtgggtcaataaaccgggggggnccgggtacccaattcgnccnaaagtggagtcggaattacaaattccactggccgtccgtttttacaaaggtcgtgactggggaaaaacctgggcggttancccaacttnaaacggcctgncaagcgcgaaattaacnntcactaaagggaacaaaagctggagctccaccgcggtggcggccgctctagaactagtggatcccccgggctgcagggttttgccgaggggtcttgggctggggcggacagtgtacgggatggaggcgactttggagcagcatttggaggacacaatgaagaatccatccattgttggagtcctatgcacagattcacaaggacttaatctgggctgccgtggtaccctgtcggatgagcatgctggagtgatatctgttctcgcccagcaggcagctaagctgacctctgaccccaccgacatccctgtagtgtgtttagagtcagataacgggaatgttatgatccagaaacacgatggcatcacagtggctgtgcacaaaatggcctcttgacatctgatgccagctctccagtggtctcccaccgggattcagtcatgcctgtctcagttaacttgtaaaactattaaagttccagaaatcgggccattcacttaatgtccaatgtggacttcttattaatatgacagtcagttaccaagacgtcagttaggagtgtggtggccttgtctgggcttttgtcactctgctctttggtgacagccactgtagtccaggatcatatccctcaggcctagaactgtgtagcccaggctgacttcaaatttatggtcttcctgcttcaaactcctatatcctggggatttagcattgtcatgggtctaggtcactttgtatatagaactttgttgtgggtcaataaaccgggggggnccgggtacccaattcgnccnaaagtggagtcggaattacaaattccactggccgtccgtttttacaaaggtcgtgactggggaaaaacctgggcggttancccaacttnaaacggcctg

SEQ ID NO: 24 - DNA, 936 bp, Rattus norvegicus
  n all: unknown
caagcgcgaaattaaccctcacgtaaagggaacaaaagctggagctccaccgcggtggcggccgctctagaactagtggatcccccgggctgcaggtctacagcgatctctcgttgatctccaactgccgcctccattcgccatggaccccaactgctcctgtgccacagatggatcctgctcctgcgctggctcctgcaaatgcaaacaatgcaaatgcacctcctgcaagaaaagctgctgttcctgctgccccgtgggctgtgcgaagtgctcccagggctgcatctgcaaagaggcttcggacaagtgcagctgcagcgcctgaagtgggggcgtcctcacaatggtgtaaataaaacaacgtagggaacctagcctttttttgtacaaccctgacaggttctccacacttttttctataaagcatgtaactgnacaataaaataaaaaaacttggacttggattaaaaaaaaaaaaaaaaaaaactcgagggggggcccggtacccaattcgccctatagtgagtcgtattacaattcactggccgtcgttttacaacgtcgtgactgggaaaaccctggcgttacccaacttaatcggccttgcagcacatccccctttcggccagctggcgtaaatagcgaagaggcccgcaccggatcgcccttcccaanagttgcgcacctggaatggcgaatggcaaattgtaagcgttaatattttgttaaaattcgcgttaaatttttgntaaatcagctcattttttaaccaatagggccgaaatcggggaaaatcccttaataaatcaaaagnatagnccggagatagggttgantggttgttcccagttttggaaccaaggagtccaccnatttaaagaaccgtgggactccaanggccaaaagggnggaaaaaaccggnntaatcagggg

SEQ ID NO: 25 - DNA, 941 bp, Rattus norvegicus
  n all: unknown
ncaagcgcggaaattaacccgtcacgtaaagggaacaaaagctggagctccaccgcggtggcggccgctctagaactagtggatcccccgggctgcaggaattcggcacgagcttccttgagactactgcgccatgagagcgaagtgggcggaagaagagaatgcgcaggctgaagcgcaagagaagaaagatgaggcagaggtccaagtaaaccatcttgtgcacccacgaagcctgcgggagcagaagtaagggatgctgaagcccggaacaagtggttggactgtatgctgctgtcggtaataagtctcagtagacccggaatgtcacctcgccgagatcagctgggaaaatgactaccttcctcacaaccaaaacagtcccgctggccctctgccctgggacctttgggcattctgggactagttctgttctcttgtggccaagtgtaactcgtgtacaataaaccctcttgctgtcagctggaagaatcaaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggggtnaatagcgaagaggcccgcaccgatcggcccttcccaacagttgcgcacctggaatggcgaatgggcaaattgtaagcgttaataatttgttaaaattcgcgttaaaatttttgttaaatcagctcattttttaaccaatagggcggaaatcggcaaaaatnccttataaatcaaaaaggataggaccggagataggggntgaagtggttggtnccagntttnggnacaaagagtccccctaattaaaaggaangggggggcctcccaaanggtcnaaang

SEQ ID NO: 26 - DNA, 929 bp, Rattus norvegicus
  n all: unknown
ncaagcgcggaaattaaccctcactaaagggaacaaaagctggagctccaccgcggtggcggccgctctagaactagtggatcccccgggctgcaggctcaggatgagagagcacgtcatgaatgagattgataacaacaaagaccgattggtgactctggaggaattcttgagagccacagagaagaaagaattcttggagcccgatagctgggagacactggaccagcagcagttattcaccgaggaagagctcaaagagtatgaaagtatcattgctatccaagagagtgaacttaagaagaaggcagatgaactgcagaagcagaaggaggagctgcagcgccagcacgaccaccttgagggcccagaagcaggagtatcagcagggccgttacagcagctgggaacagaagaaattccaacaagggattgctccatcaggggccggcaggagagctgaagtttgagccaaacacataaaagtcctgatgtctgccagaacttgggaagaaaaccgttgactcaacatctgtttcatctttcaacatcccttcttttctcttcactcaataaatactttaaaagcaaaaaaaaaaaaaaaaaaaaaaactcgagggggggcccggtacccaattcgccctatagtgagtcgtattacaattcactggccgtcgttttacaacgtcgtgactgggaaaaccctggggttacccaacttaatcgccttgnagcacatccccctttcgccagctggngtaaatagcgaagaggcccgcaccggatnggcccttcccnaacagttgngcaccttgaaatgggcggaatgggcaaattgtaagcgttaanattttgttaaaattcgcgttaaatttttngnnaaatcaggcccanttttttaacccaatagggccgaaatcggnaaaatn

SEQ ID NO: 27 - DNA, 921 bp, Rattus norvegicus
  n all: unknown
ncaagcgcgaaattaaccctcactaaagggaacaaaagctggagctccaccgcggtggcggccgctctagaactagtggatcccccgggctgcagggagaactatctcgagttttttttttttttttttttttaattttgagactgggtctctctatgttgtccaggctagtcttgaacttctggattcaagtcatctacttgtgtcagcctcttagctcctaccaccacacttgactttgcttgtaactttgaaaagtccattcaaaattaagctcttaagagactgaatggaaaggcaattttgtctgaaggatatttcctatgtaagggagaatagcatttgcagaatataattctggtgctgctaggggaaaaatcagtaggaagttatagttcccagttggctttaaccaactacaaccttctctcaatataaagtattcaagaataaagagtatggtatctacttatcagaaaggcatgtttcctattgggcaaagttagtgaaaaagtgactttactcattttgcatttacctcggctgtataagcatttcctagcgcaggatgcttcttccagaaatcaagaaccaggtgaatacaggactaagaccttcctggatgttcttcccacatctagtatgttgaccccaacactgaacttggcaaatcttaagttgaccctggaatactcaggcttccccnatttcccttcagctgataacagaatcntttggaaagctctcagcagatccgnanagttgcttacccgataataaatgcatatcaaagcctttaaaggaaggaatccnangccaaaggatccancccttnggnnttacnaaaggntacctaggggggattaanggaaaaaaggnttggcccccccaaggtccttcccagntncnggggaggnaana

SEQ ID NO: 28 - DNA, 925 bp, Rattus norvegicus
  n all: unknown
ncaagcgcgaaattaaccctcactaaagggaacaaaagctggagctccaccgcggtggcggccgctctagaactagtggatcccccgggctgcaggtgggaagatagtcttaagaataaccttttaatgaaggagttggcaaatatttcaagttgtgcctgctggttccagggttcttaacctctctagttaagggcttagctttcttgggacatcaactgtcttatttctgaaaaagaccaaatgtaactggtgtcaccagcagtgtgggaatgaccaagtatgactttgtccctgtgattcaaaagatgtttgtcaggtagagttgggtgaatgccattattgtgtgcatgggtatgtatgggtgggatatggtctcctggcagactggaaataaatcagagcaatttaaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaanagttgcgcacctggaatggcgaatggcaaattgtaagcgttaaaatttgttaaaatcgngttaaattttgttaaatcagctcatttttaaccaataggcgaaatcgggcaaaatcccctataaatccaaagnatagaccgngataggggtnagtgttgttccagtttgggacaagagtccccctatttaaagaaccgtgggctcccaaaggtccaaanggggggaaaaaccggcctaatccangggcgatgggcccactaacggggnacccatcaacccnnaaancaaggtttttttgggggcccaaggtgc

SEQ ID NO: 29 - DNA, 918 bp, Rattus norvegicus
  n all: unknown
ncaagcgngnaaattaaccctcactaaagggaacaaaagctggagctccaccgcggtggcggccgctctagaactagtggatcccccgggctgcagggttctgatggtataagcaaaacaaataaaacatgtttctaaaagttgtatcttgaaacactggtgttcaacagctagcagctaaagtgattcacaccatgcattgttagtgtcacagactttgtggttatgtctaatagctgtttctgaagtattttcgtttatcttttgtctaatttaaccctaagtgaattctctcctttttcttgaggacacacttatgctcaaagtgttgactctgccgtagtggcataaagagagtgtaccgtttgacagagatgcaaagttcagcagtggacctaaccagatgtcctgtggctgggatctgtgctagcagtttggagcacgagctgtgtgcctgtgaactggaatgccacttgtcccactccatctacgccttgcagaatcagttccacttgttaaaggcaaaggctacttaccaccttaatgctattttctgtaaagaaattaaattttacttttagccttttgcaaactttttttttccaagccggtaatcagccactccaaaacaactattctcagatattcatcattagacaactgggagttttttgcnggttttgtagcctactaaaactgcttaggctgttgaacattccacattcaaagttttgtagggtggtgggataatgggggaaacttcaatgnttaatttaaaaataaataaaataagttcctggacttttaaaaaaaaaaaaaaaaaaccccgaggggggggccnggnacccaattcgncccaaaaggggggccggatnacaaattcccngggccgccggttttaaaacggncgggaccggg

SEQ ID NO: 30 - DNA, 918 bp, Rattus norvegicus
  n all: unknown
ncaagntcgaaattaaccctcactaaagggaacaaaagctggagctccaccgcggtggcggccgctctagaactagtggatcccccgggctgcaggtcgagtttttttttttttttttttttttaaaaggtgagtcaagatacacagctttaatacatattagaaatatccaatgtgccaccaatacgattcccctaaaacacagcaagtgccagcgcttggggccacactcatctgtctttgtatcactagacatctgaatgaccaaccatccatttttcccacatcctgccattcattaaggtattttcagccagattttttagcaatatgctttttttctttctttcaaatacaacaagccacacagggagttctactatggaatgtccaacaacaacagggctgtatgggggccaagccttttctggaaaaacatggcggatctctaaaagattctctgtcttccctttatggagtcagcagtgctccacgttaattaagccacttcaatttactgtatcagtttggatattcgttttaattgtgggactagacacagaaactcacatttctggccttttcctctgcatttctcaatatactatgggtttttttttcccacaccgtaaatacagcatggattgacaggtagaaactcgtgtcaatagtctgtgggntttatgccaactcagtggagtgatactatatattantnccagntccctcncaaggcctanantaagatgnngnaatagttgcnatggtgggtaaccttcctggcggttaagagaagtgacggcancctgnccttagatcagaaggtaaaaacccccaattggccaaggaaaaggccggcccaggngggaccggncaggntnaaaggaaangccttaannaaatgggaacccccggnng

SEQ ID NO: 31 - DNA, 925 bp, Rattus norvegicus
  n all: unknown
gcaagcgtcgaaattaaccctcactaaagggaacaaaagctggagctccaccgcggtggcggccgctctagaactagtggatcccccgggctgcaggcttccctgacccacagttggaccgggcattgtagccagggtccgtcgcacttttcggtggtctgcacggactgagccaactcggtgtggacgatctcctggctgtggctgtgtggctagtccacctttgcagttccagtagtcagactggagtctctttggaagcagctctaaggaatacaccaggatctcagggtttatctgtgtagccctggctatcctggagctgtctctgtagaccaggctgggctgagactgaggcccagttgcctctgcctcttgagtgctgggattaaagagtctcaatgtcttcgctgacgctcctttggatgtacccccaaatctctggaccacatcaccctgggacccccgatgcggctgcctgagcaacctgggagatggaaagcctgagaatgagacaaagggggaccaagaaacccccgaaaggggagaggagccacggagaagcccagcccctgacttccccacctgggaaaagatgccgttccaccatgtaactgctgggcttgttgtacaagggaattacctcaaccgntcttctgtctgcaggcagcgacagtgagcagttgggctaatatctctgtggaagaatcgatgataagagtcaaaatcgttcaaaggaaagctgggcttggtggctgtccactggaancccagtatccaggggactaaagaccaggagctgatgccggnttccantacccagggggnaattgtcctttggaaaaccaacgggtgaanaatgtaagccccgtggnaaaaantgcnggggccttgtgctgcaaaaaagcnngtttaatnaaanncc

SEQ ID NO: 32 - DNA, 921 bp, Rattus norvegicus
  n all: unknown
tntgcaagcgcgaaattaaacctcactaaagggaacaaaagctggagctccaccgcggtggcggccgctctagaactagtggatcccccgggctgcaggtgggcctcgtgcgtttgggtgtgtggtataactccttccgggcctggaagggaggcttctctggaaactttgaaggcgaaggcttcatcctcggaggggtttttgtgataggatctggaaagcagggcgttcttcttgagcaccgagaaaaagagtttggagacagagtgaacctgctctctgttctggaagccgtaaagaagatcaagccacagaccccagcctccaggcaaagctgatcacctgctggctgggggggggggaacgggggcctgtgcagtgttcaccagatgagctgtgctttcactgtgaccccaagagctaggaggccattgcaccatatttactgggaattggtgatgtattttaaaattgtctgtttaggtcccagaatgtttaacattccgtttagacccaatagggcaaataggtcccagacagaacagagtaaaatctaacaaatcagtgagagttatttgaggaaagatctagaaaatttaaggcctaaagttgactgttaagcctcccgttcacaggaatatgtcctaagtgccagggatgtgaagtagaggaagntttcatgcctaattaaaaagaaaacatctgaaatctgagaaaagtggggactaagaaacaactacaactccagtggtagagcatttacctaacgtgcacatggncctgggtaggataccccagaccagaccagaccattcacaccacctaagagaagctgatgggttgacttgataattaggggaatatcctaaagccaattgtgccggngttcctnggacagtttggccaanggnaaaattccaa

SEQ ID NO: 33 - DNA, 933 bp, Rattus norvegicus
  n all: unknown
ncaagcgcgaaattaaccctcactaaagggaacaaaagctggagctccaccgcggtggcggccgctctagaactagtggatcccccgggctgcaggcgtggtcgcgctcgcgtgctccgttccctgcggctgcccggacccttggccatgtcctgaatgggaaacagcacatcctcgttttgggggaagtcagccactactcctgtgaaccagatccaagaaacaatttctaataattgtgtggtgattttctcaaaatcatcctgctcatactgttcaatggccaagaagattttccatgacatgaatgtcaactataaagtcgtggagttggatatggtggaatatggtagccagtttcaagaggctctttacaagatggactggagaaagaactgttcccagggatatttgtgaatggaatatttatcggaggtggcgggccgacactcacaggcttcacaaagaagggaaattgctggcctctggttcaccagtggctatttaaacaaaagcaagaggaaagacgtcgaatgacatggctagtcgccgtaccagtaaacgttagtgcagtcataacctttcacttgaggatgttttcagtgtgtgggatgccctcataaagatgaaaataatgaacaataaattgccatggacccctcaaaaaaaaaaaaanaannnnnnnnnnnnnnnnnannnnnnnnnnnngggnnnnnnnnnnnnnnnnnnnnnnnnnnnnnnnnnnnnnnnnnnnnnnnnnnnnnnnnnnnnnnnnnnnnncncgggggggggnnnnnnnnnnnnnnncccnnnnnnnngggnnnnnnnnnnnnnnnnnngnnnngnnnnnnnnnnnnnnnnnnnnnnggggnnccccnnnggnnnnnnnncnnnnnnnnnnnnnnnnnnnnnnnnnnnncnnnnnnggggggg

SEQ ID NO: 34 - DNA, 945 bp, Rattus norvegicus
  n all: unknown
agttatgcaaggnngaaattaacccgtcactaaagggaacaaaagctggagctccaccgcggtggcggccgctctagaactagtggatcccccgggctgcaggaattcggcacgagggcggccagaagaaggagagacttcggagcacaatgccagcatggactttgcagaccttccagctctatttggggccactctgagcgatgagggactccaggggttccttgtggaggcccacccagaaaatgcctgcagtcctattgccccaccaccctcagccccagtcaatgggtcagtctttattgcactgcttcgaagattcgactgcaactttgacctcaaggtcctaaatgctcagaaagctgggtatggtgcagctgtggtacacaacgtgaattccaatgaacttctaaacatggtgtggaatagtgaggaaatccaacaacagatctgggatcccatctgtatttatcggagagagaagtgcagagtacttacgagctctttttgtctacgagaagggggctcgggtgcttctggtcccagacaatagcttccccttgggctattacctcattcctttcactggggattgtaggactgctggttttgggccatggggaacagtattgatagttcgttgcatccagcaccggaaacggcttcaacggaacagacttaccaaagagcaactgaaacagattcctactcatgattatcaaaaagggagatgagtatgatgtctgtgccatctgtctggatgagtatggaggacggggacaagctttcgggatacttccctggtggcnccaaggcnttaccaacagtcgctgtgtgggancccctggggtcaattcagaacccggcaagaacctggccccaancnggnaaaanaagcctggtccaaccggggggggccttggggggatnaagggaaaaaggnan

SEQ ID NO: 35 - DNA, 975 bp, Rattus norvegicus
  n all: unknown
gtcgttgttnntngnannggtnnnaatnaacccncacgtaaagggaacaaaagctggagctccaccgcggtggcggccgctctagaactagtggatcccccgggctgcaggctcctcttcttcctcctcttcctcctcctcttcctcctcttcctcctcttcttcctcctcttcctcctcctcttcctctctctgtgtgtgcgtgtgtgtgtgtgtgtgtgtgtgtgtgtgtgtgtgtgttattatctaagtgaatgtgtatttaccatttcttttatgaaaacgaaccccacactgtttgtctccctggtaagtagtatctccagttaaatgaggcccctccctgcctctgcttctgagttcagtcttagaggaccagggatttaaacaggttgcagtggacacagtcttccctaccatgttctccacctcatccagtgtacacgcacatagccgtcctttctaaaacaccaagaaccttggaattgcgtgagtctcccctagcttctcaataaacactgatttttttttctccagaatctgaaaactaactacacaaggaaattattttcaaatggctgctcagttttggtggcttgggctatatacactgtcccaaaacctggctggactttnaaaaganacatatactttaaatctaaagcacttncacacaaganatccagagattcacaatcaaaaggggacactgatgtgggaattctccaaaatactcaaaaaggcatgaatttgtnttgaatttggtttctgggagaattctttctttccttcataataaaaatagctccnatngaagggctggaataagnaaacggaacaatggcaaaggcctaagtncaaaggggggggggnccggggnacccnaaanttcggcccctaanaaggtggaagccgggaannnancaattccaactggggccgggccgggttntaaanaanggccggtggaacntg

SEQ ID NO: 36 - DNA, 1036 bp, Rattus norvegicus
  n all: unknown
cgcacattaaccctcactaaagggaacaaaagctggagctccaccgcggtggcggccgctctagaactagtggatcccccgggctgcaggctggagagatggatcagcagttaagaacactaactgttcttccagaggtcttgagttcaattcccagcaaccacatggtggctcacaaccatctgcagtgggatccgatgccctcttctggtacacacaactacagtgtactcatacataatanataaacctttaaaaaaagatacataactgcaagtaattaanaaaaaaaaaaaaaaactcgagggggggcccggtacccaattcgccctatagtgagtcgtattacaattcactgggccgtcgttttanaangtcgtgactgggaaaaccctgggcgttacccaacttaatcgccttggcaggcanatccccctttcgccagctggggtaatagcgaagagggcccggcaccggatcggcccttcccaanagttgcggcagcctggaatggcgaatgggaaattgtaagngttaatattttgttaaaattcgggntaaanttttgttaaatcagcnnatttttnaaccnntaggnggnaaangggcaaaannncntaaaaatnaanggntttgcancgagtcanggttnngccatntnncagattgggcnaaaaagnccnccaccgatagnccntggcaacaantgcgnaccgggaatggcgaatggcaaatngtaagcgntaanaatttggttaaaaattcgcgtnaaaattttgttaaaatccagccccantttttaaaccaatagggccggggaatccggnaaaanggccccnnnngnaattcaaaaagantaaanccggnnaaaaggggtttaaatngnnggtncccanttttgggaacaaanagnncccccnnatttaaagnaacnngggggcccccaacggnccaaaaggggggaaaaaacccggncnaantaaggggngnannggcccccctaaanggngaacccatnnncccccnaancaaaggg

SEQ ID NO: 37 - DNA, 1023 bp, Rattus norvegicus
  n all: unknown
ncaagcgggaaattaaccctcacgtaaagggaacaaaagctggagctccaccgcggtggcggccgctctagaactagtggatcccccgggctgcaggttctatacattgcctacagtgatgaaagcgtctacggtctgtgaagttgctgtcccggaggtgggggttccattctacaaagagaggtggcgctccttccttggcatccagttcctccttcaggctcaaacaccatctcctttcttcaggacctgcacttaatgtttgaggctgtctctccagtccctctgagcaggaggggtaatggtagatgtacagcggggggggcccggtacccaattcgccctatagtgagtcgtattacaattcactgggccgtcgttttacaacgtcgtgactggggaaaaccctggcgttacccaacttaatcgccttgcagcacatccccctttcgccagctggcgttaatagcgaagagggcccgcaccgatcgcccttcccaacagttgcgcacctggaatggcgaatgggcaaatgtaagcgttaatattttgttaaaatcgcgttaaattttgttaaatcagctcattttttaaccaataggccgaaatcggcaaaatcccttataaaatcaaaagnatagaccgagatagggttgagtgttgttccagtttggaacaagagtccactattaaagaacgtggatccaacgtcaaaagggcgaaaaaccgtcnannaggggggatggcccacnaacgtgaaaccaattntccctggngggaagangttttggggnaaggaagtaaactgggggaancccttaaaagggggggaccccgaantttggaggccttcnagggggggaaaagccgggngnaaaggtgggggnngaaaagggaagggggaggaaaagggaaaagggaanggggggggtaaggggggtttgggaaaatttaanggggtaaanttgggggggaaaannaanaaaaaacngggggggggtttaaatnggggggtaaaagggg

SEQ ID NO: 38 - DNA, 979 bp, Rattus norvegicus
aagcgcgaaattaaccctcactaaagggaacaaaagctggagctccaccgcggtggcggccgctctagaactagtggatcccccgggctgcagggagtaatggtggagcagaatggatgccacttgtcactgtgctcgatgacaagttgcagcccataaaaaggttgacttgcttgcaaacacattgtgttcgttggcattttctcagcttctcctcactacctctgggtggagatgggcaccttctgtgggcctgggctgggggccacccctgctatgcaatggagaggcaaaggcagaggtccaggaataaggaggcttctaccaatgattttgtttaatggtgcttgacagagatattgtatggttctctggagagctcccctggaaaaccttacctccaaccacacaagggcttcctcccagagagcgctcgctgggcagcaagggacacactcccatacttgccaagcatatcaagtacccaaagattggcagaaaagatcctggcctgaccacccagccacatccttcagggctccaccggattgactgtgtgtctgagatggagagggctttgtgacatttaagtgcctttcagaaatgccttatacggtgagaagccaaaggtttatgtcagcatggcagagctcctgagaccgaagccttcctggagcctttcgttactggcagcgttctttccgaagccaccggggtncattccacagatcgtattaaggaggagctcnacaaaanctcgtggggcnagttttcagcaagggcgatagnngntgcttgcaaccatgantccnagcaactggccnnnngaannagtnggaaanaaannancccggnagcantcnagggggtntaagnanaggggncaanccanggnnnngnantgggaanttgggatgcgatngnaaantnccggnnaaanccgggttgaaancgganagttgaaaaangggtcgggatt

SEQ ID NO: 39 - DNA, 1112 bp, Rattus norvegicus
aaacggcantanccctnactaaagggnacnaaagctggagctccaccgcggtggcggccgctctagaactagtggatcccccgggctgcaggttgaatattaactcgtgccagcaggtgaaacaaaaagaaaccttctgtcgtcgtagaagaatatttcgcccaggctgtgcgacgacattcacagcatttcaaaccagaccatctctgtaaatagctgagtgcctaataaaccattattttggtaaaaaaaaaaaaaaaaaaaaannaaaaaaaacncgngggggggcccggtacccaattngccctatagtgagtcgtattanaattcnctggccgtcgttttanaacgtacgtgnactgggnaaaccctggggttacccannttaatgcgccttngggtnanntccccctttgcgccagctggngtgaataagcgaagaggccnggancattggccnttncccanaaaattgngcnnnnnnatnggnaaanggnaaattnngnggggtaanaatttgngtnaanagnngcgcgtnaaannttnaggngaaangcggngcanttntnagcnaaaagggccaaaaaggggannaaancgccngangatgagaanaggataggacgngnngaanngnnagggatgagggaganaatnnngnaanaangggnacngnnaggaagaaaggnnaggngnaagtgganaganngacaaagtngagagaagnanagngggagangagggacggagagggaanaanngagaganngnggagntanncgggaggtnnangnggntnnggagagnagagngngnanaggnngaggagangagagggngganggaagagacgaaagngggaggnnnannnnggggatggggagngnnnggancagngnaggggangacaggtnntggantgggggnagaatngagantgtgnagngagggntnnatatagagaggtgnagagaantggggganagntgngacnnngagataaggagaagganacngacgaganggngaagnaggnagagtantgangaaagaaanacgagagaagagagtnannancntagatanacgaggngaagnnnagnnacgnggtc

SEQ ID NO: 40 - DNA, 1026 bp, Rattus norvegicus
aagcgggaaattaaccctcactaaagggaacaaaagctggagctccaccgcggtggcggccgctctagaactagtggatcccccgggctgcaggggacgagatgctcagcatggtgagtgaaggggagggaaaacccatgagagagtgagatggtcagagaatgggagctgattgtgacatggaactgcagagagaagcacagacttgaaacatcgctaagatgtgtgcatacaaaaatgaagcaagttatgctaagtacacacagtgtccagcacattttattttcacttttggttttgaagacaaagtctcattatgcagaccaggttgactttgaattcagatctgcctgtctctgcctctggagtattaggatgaaaggtgttatgtcaccatgcccagcctcttagtatatttcagaacagtaaatactgcatgaaaggtcattgtaaattcccctcttaattattgcttcaatcaagttggaaatgctttcatgtattaaagacaatgtttttaatggcaagaaaaaagtaatgttttatttttatagtttataagccatgcattacnatttttatgtaaaaaaaagnactaatgtagaatttnggccgaatataaaagtggngttgtgatanaattaaaaaattagggggcnggggttnagcccaatggganagcgcntgnccgaaggaagncacaaggncncggggtangggnnccccagncnccggaaaaaaaagacccnnganaanaaganangaaaaaacnccnagggggggggncccgggtnanccnaaantcggccccnaannggggaagnccnaaagnannaanttcncnngggncggnngggtttaacaaanggcggngggcnngggggaaaaaccccggggggannnncccagcgnganttnggcnnggnggggggnaanccccccnnnnnnngccnggnggggnggnnnatnggngnnnggaanccnngcgnnnngaaagnngggannnncnaanngaanngggggncg

SEQ ID NO: 41 - DNA, 1044 bp, Rattus norvegicus
aagcnngaaattaaccctcactaaagggaacaaaagctggagctccaccgcggtggcggccgctctagaactagtggatcccccgggctgcaggcgagtttttttttttttttttttttttttttttttttttttgatttttatggaaattttaattggcaaatttaaaaaaataagtttgtaaccattattttatatagaaatattcaactttcccaagatttctcacaaacagnggtacaaaagttggctctaaattcatccaaggtattttaagaactaaatggncttgcacttgattgactccagtctcagtgatgctgggaaggaagcctaggaccttgcacatgcncagtaagagctttaatgccaagccacaggcccattccncagttgacnccttatcaataatcttcatcttgggagttttcnccaagaatcaattcacagggntgttcagtctttctctacctcaaccctacccagtgngnctaaatcancagtttagtccatttcgggaaacaaaccacttgtcaaacgnggaaatgaaatgaagagatcttagtagtcaggnattntggtaccancccaactgggggggncaatagtagaaatggctgtaaacaaaagngaatctaancnaaggggggggcncggtnnccaaanncgncccaaaangnggagngcggaaacaaaaaatcngccgggnggnccgttataananangttggggganngnggnaaaaaccctgnggtgttngcngaaantnattcggccggtggggggganaaaaccacnnccttgggancngggggggaaaaaaagagaaaaaagncccnganggggggggcccggttancccaaattcggccncnaatnggagnaggncggaaatgngaaattcccntgggccggncggttttnanaanggnccgggganctggggggaaaaancccnggggggntaaccccaacccttaaancggcccttngnggnganaatnccccccttttggncaaggcggggnggnaaaaaagcggag

SEQ ID NO: 42 - DNA, 997 bp, Rattus norvegicus
aagcgngaaattaaccctcacgtaaagggaacaaaagctggagctccaccgcggtggcggccgctctagaactagtggatcccccgggctgcaggcttcttttagtgccagctcagtggctttatcgctcaagagagacagccggtaacagataggctgcccctctgctcacttttctgtttcacagacacaaggtgtttttgtcccaagaaagcctcctggcttagctgtgtgactaaatgctatttgccctcttcagtggacctctattctcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcacctgaatggcgaatggcaaattgtaagcgttaatattttgttaaaattcgcgttaaatttttgttaaatcagctcattttttaaccaacaggccgaaatcggcaaaatcccttataaatcaaaagaatagaccgagatagggttgagtgttgttccagtttgggaacaagagtccactattaaagaacgtgggactccaacgtcaaaggggcgaaaaaccgtctatcaggggcgatgggcccactacgtgaancatcaccctaatccaagtttttttggggncgaggtggccgtnaaaagcnactaaaatcgggaacccctaaaagggagccccccggtttagaagctttnaaggggggaaaanccngggggaacgtgggccnagaaaaggnaggggnggnaaancggaaaggggncggncgctnaggggannaggccaagggnnaannggntngngntgnggggnannccnnnnnnnannccnnnngggggngaaaanncgggggnaaaaacgggngnnnnaaggnnnggg

SEQ ID NO: 43 - DNA, 1019 bp, Rattus norvegicus
aagcnggaaattaaccctcactaaagggaacaaaagctggagctccaccgcggtggcggccgctctagaactagtggatcccccgggctgcaggcgaactccagcacttctgctcttgtttttgttttgttttctgcgtaaacctctggcccactctcaaaaggcaagatgtccagtcatgtcccggcggatatgattaatttgcgcctcatcttggtgagtggaaagacgaaagagttcctcttctccccaaacgactctgcctctgacatcgcaaagcacgtgtatgacaactggcccatggactgggaagaagagcaggtcagcagcccgaacattcttcgactcatttatcaaggcagatttctacacgggaaacgtgcaccctagggagcattaaaacttccttttggcaaaacaacagtgatggcatttggtggccagagagaccctggccagagcccaattcacaaggtcagagaaaccgggagaaaactggtgagagcaactgctgtgtgatcctgtaacatcgtcgccagcgcagtgtggcagtctgttaccactgcggggacagaggagactcggcagcttccgganacctgtgggacagtcgcccgcacatcnaggactgaaccactncatgagctctgtgatctctcctcacaaagttaaaaggaaccaaggaacatttcncagttctggtcctttantccngtnnctcttgtctggtgtttgagccantctgnaaatggcacagggggtcttcnaagggggnaaattagcgaagtcttctnaagggggggtttctgnaaggggggggggcccgggtaanccaaatttnggccctaanaaggnggngnnggnaattnacaannttcaactggggccggnggtttttaaaaanggtcgntggnnnnggggaaaaaccttgggggggnancncaaanttnnaanngngntttnnggggnaanncnccnttntggaaagngggggggaaatttgggnaanaanggggggg

SEQ ID NO: 44 - DNA, 952 bp, Rattus norvegicus
agctngaaattaaccctcactaaagggaacaaaagctggagctccaccgcggtggcggccgctctagaactagtggatcccccgggctgcaggcgagttttttttgtggtttggtatttatttgaaccacgtgatctcgtgtagtttgggctagcctcaatttaaacttaaactcctaaccttccttcctccactctgagtgaggggcttgggggatataccaggctctaattctttttactttacttttttagatgtacttacgtcactttatgtgtatgaacgttttgcctacatgcatgtatgttcactgtgtctgtaggctcctcctaggattacagacagttgtgagccaccatgtggtgtctggggaatggagtctgggttctcttcaagagcaacagtgttctcggcccctggaagtcaggttctaatacctgttaggtaagcagtgttgggctgatcagatgcaaagtgatttagcccctatcataacagactgtcagtctcggcctccaggcactccaccacctgctactccagttgaagtgtcctgccaggtgaccttggctgggctatcggatcatgtgaaatacagaccctgctcaaaggaacaagcttgcgggntggagagaggctcancggttaagagcacctgactgctctccagaggtccgagtcaatcccagnaaccacaggtggctcacaancanctntaaagagatccgaagccnncttctgggggaacngaagananctacagngnactanannnaanaaaaggngaannaaacntnannaaaaanaannnnnnnnnnnnnnnnnnnnnnnnggnnnnnnnnnnnnngnnnnnnngnnnnnnannnnnnnnnnnnggggggggggggnngnnnnnnnnnnnnnnnnnnnnnngnnnnnnnnngnaaaannnnnnggnnnnnngggnaaaatg

SEQ ID NO: 45 - DNA, 993 bp, Rattus norvegicus
aagcgcgaaattanccctcacgtaaagggaacaaaagctggagctccaccgcggtggcggccgctctagaactagtggatcccccgggctgcaggcaaactggatgaaactttgttctaaggggaatttcatttaaaagtttacctttaccagagcaggaggcgtagagtcagctctggggaaggagtgggtaacttcacgacactctcattctccgcacttactgctccacctgagtagctgtaaaggaacttgggctgggatggggtggcaggcagtgtctctccttcatgggcctatggctgaatcaaacaatccttccatagcacatgcttaaccctggactcactcttaagtcccttctttcccattctgctacaaagtcaggctccctaataacatgtaactggagctgccttgtcaacagagaaagaagaaagctaacgaatacccatgatcctattcttcaccgtccatgtctcgatgctccatctccttcctggatcctcttgttgctttctagaattttcaccaactatcactcganttantagtccaatctgtcttgaaagaaaaataaagttgaacaaagcaacaaaanannaanaannaaaaaaaaactcggagggggggcccggnaccnaattgggnctannagngngggggnaataacaatganggggggtngtttnnanaangnnggggntggggaaaacnctggggtngancnnaattaatgggnctngnaggnaaagggccnntttngggggagggggggganagaagggnagggggnccggnannggngggggcnnnngnnagnnntgggggaggnnggantgggngaaggggngnaagngnganngnggnnaagnattnngngaaaaaanngggggnnaaaatnnnnggnaaanngggggggnantgggntaangnannggggngnanannggggagaaaangggngnnnggaagnnnaaannggnggncggg

SEQ ID NO: 46 - DNA, 1033 bp, Rattus norvegicus
aagcgngaaattaaccctcacgtaaagggaacaaaagctggagctccaccgcggtggcggccgctctagaactagtggatcccccgggctgcaggcaagtttagtcctagtgcccacatnaagtggttcacagctgccctgtaaccccagctccagaagatccaagaccccctctagcctctgagcacatagccccatgcatacacacatcacatacatgatttaaaattaagtaagctttttaggccttatatttaattcacctattaaatgcttagacaccttcaagaaatttggcaagtttgaagtaataagggaaggaaatgagtattggttgagtaaaacagcctcaagacagacacctgggtcaaatgtatgtggcagcagcatgccaaggccctagctccagnttactggtgagaaactggagcttgagagagaccacataacctgggagtgagtcataatgaaaaccaagtggcagacctgtttcaaaagtataacctcaggggttggggatttagctcagtggtagagcanttgcctagcaagcacaaggccctgggttcggtccccagctccgaaaaaaaanaaaaaaaaaactcgagggggggccnggtnacccaattnggncctanngtgngncgtattanattnantgggccgggcgttttaanaangtcgngacnggggaaaaccnngggggttngccnnanntanangggngtgggaagcgagatncggccntgtgggggagntggggnnngataggggnngngggnncnngnnnnnggantcgggccnntgnnnnanggagnnggggnnggnnggaantggggnananantgggananttngganngngngtnaggnnnnngnggnggaangagttgnggggttgaagntntgnggggaggaannnggngnggganttntggaaancggaggaaggnggngaaanggggngagnanngcnggngaagangngaanaaggnnngggncggggganngaggggnnnggnngttttnttg

SEQ ID NO: 47 - DNA, 1005 bp, Rattus norvegicus
aagcgnggaaattanccctcactaaagggaacaaaagctggagctccaccgcggtggcggccgctctagaactagtggatcccccgggctgcaggaggctgccgtttntgagtttnagtccttgagaggctgggaaggcacagagctctggctctgcactgttcttactgagtgactaggtgtgagccctttacattagagggaacctggtttgagctcacttgtactttgtgtggcgttagtgttccattactggcccctctaagtaatggtcttcacagtgcacagcaagttcccagtgtgtagaaagccatacaccaggatgtgggtcaaccatgaagatgtggcattgcagacaggggaacatgtggatgcatggntatcaccttgagcagcccctgcagttgcttgtgttaacacaaaagtgtttagcattctgccgnttttatatttatgtaataactctttaaagccattcagatggataactatttaatttcttaaagacagttgtaaaggtctctctctgaggacaatgacttggtaaaactgggggcacagccagtcccagacactggtcgtggntacagtgggntttttgggctcaggntcaacacgcatcagagtaggactggggncaacangtggtgggngtgtgcaaacaggnnggcnctngancagcccaggncctttggagagcacgtnctctggcaccaaggnccctcngntntgggaagggggaaaactttcacaagggaaatgggngncaagcttttannccncngaaggncntgggnggggggcangggcaagngggggcgggngggggacnnntgntttggggggnannttntgggggncngaggggnnnaaanccgcnccctgnaggnnggcggaggngggtgnnannnaccngttggagnaagagcgngggggntnaagggnggtggnaaggatgtgggncggaacgttgngggaagtnggaagagnnagggnganccgcgg

SEQ ID NO: 48 - DNA, 975 bp, Rattus norvegicus
aagngcgaaattaaccctcactaaagggaacaaaagctggnagctccaccgcggtggcggccgctctagaactagtggatcccccgggctgcagggtcatacttaggaatttctcctactctacactctctgtacaaaaataaagcaaaacaacaacaacaacaacaacaacaacaacaaccataccagaacaagaacaagaacaacaatggtttacatgaacacagctgctgaagaggcgagagacagaatgataatccagtaagcacacgtttattcacgggtgtcagctttgctttccctgaaggctcttggtgacagtgtgtgtgtgtgtgtgtgtgtgtgtgtgtgtgtgtgtgtggtgtgtacttgtttggagaagtacatgtgtacacatgtgaggacctgggggcacctggaccagaacgaacaagggcgaacccctttcaaatgggcagcatttccatggaagacacacttaaaacctacaacttcaaaatgttcatattctatacaaaagaaaaatagataaatataaacattttgaagttgtagcatttccatgaagacacacttaaaacctacagggggggcccggtacccaattcgccctatagtgagtcgtattacaatcactgggccgtcgttttacaacgtcgtgactggggaaaaccctgggcgttacccaacttaatcggccttgcagcacatcccccttttggccantggngnaatagcggagangcccgcaccgattggcccttcccaacanttggcggcnnctgaaatgggcggaatgggccaaatttgttaaggcggnnaaaaattttggttaaaaaattngcgggtnaaaatttttgggaaaaaccagcccnattttttnaanccaatagggggggaaattngggaaaaaacccccctnataaanncnaaanggaatnaggccccggngaaanggggttgnaattgttgttc

SEQ ID NO: 49 - DNA, 949 bp, Rattus norvegicus
aagctcgaaattaaccctcactaaagggnaacaaaagctggagctccaccgcggtggcggccgctctagaactagtggatcccccgggctgcaggattagacttaccgctaccaaacaattctttacttagattataggtgccctcctcccattgttagcatgggggatattaggataatatcactttaagataacacatgaggggttggggatttggctcagtggtggagcgcttgcctggggagcgcaaggccctgggttcgatccccagctccgaaaaaaaaaagaaccaaaaaaaaaaaaaaaaaactcgagggggggcccggtacccaattcgccctatagtgagtcgtattacaattcactggccgtcgttttacaacgtcgtgactggggaaaaccctggcgttacccaacttaatcgccttggcagcacatccccctttcgccagctggcgttaatagcgaagaggcccgcaccgatcgcccttcccaanagttgcgcacctgnaatggcgaatggcaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccatttggnacaagagtccactattaaagaangtggactccaacgtcaaagggcgaaaaaccgtctatcaggggcgatggcccactacgtgaaccatcaccctaatcaagttttttgggggtcgaggtggccgtaaagcnctaaatcgggaaccctaaagggggncccccgatttagagccttnangggggnaancccgggggaaacgtgggcggagaaaaggaaggggaagaaaaccgnaaaggnancnggggcgctaaaggggnctgggaaaattgtancgggnn

SEQ ID NO: 50 - DNA, 958 bp, Rattus norvegicus
aagntcgaaattaaccctcactaaagggaacaaaagctggagctccaccgcggtggcggccgctctagaactagtggatcccccgggctgcaggaattcggcacgagatcatggctgcagtcagatctccgttgtctctgtggaggttccagttgagcactcggcgagcacggcgggtctgtactcgggtcgcagcccagcgccactccgatgctctgctcgcgacgtggtcccagccctttgaagtggggcagcctcgccgccctttcagctccgaggcagaatctggtagctcaaaagtcaagaaacctacttttatggatgaggaggtccagagcatcctcaccaagatgacaggcctggacttggcagaagactttcaagcctggctgtacaaccactggaagccaccaacctacaagttaatgacccaggcacagctggagggaggctacgagactgggcagttgaggcagctaaagtacgattaaagatgccaccagttctgggaagaacgaaagccaataaatgatgtgttagccgaggataagatcttggaaggaacagaaacaaacaaatatgtgtttactgacatatcgtataacataccacaccgggaacgttttattgttgttagagaaccaagtgggcacactacgcaaagctttcatgggaaagaacggggacanggtgatacaaatttatttcccgaaagaaggtcgtagagttttgccaccagtaatttttcaaagntgagaaccttaagaccatgtacagccaagaccgggcatgctgatgtncctcnaatctctgtgttgcccagtttttgagccagattccantggggtatanccaaggggtnntcacccagacccnnnggaggntttnngnccggncntgggnaaananggggttnttacggggccaanaanggcancttttgggggggaatggggtg

SEQ ID NO: 51 - DNA, 979 bp, Rattus norvegicus
gcaagntcgaaatnaaccctcactaaagggaacaaaagctggagctccaccgaggtggcggccgctctagaactagtggatcccccgggctgcaggagatcaaacactcctggttttgatctgtgagctcattatcacatgttagggaagaancaaactgtgataatgagctcacagatcaaaaccaggagtgtttgatgtttgcactaggagctcctgaacaaataaagtttagcaattgcagcataaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgttaatagcgaagaggcccgcaccgatcgcccttcccaacagttgcgcanctgaaatggcggaatggcaaattgtaagngttaatattttgttaaaattcgcgttaaatttttgttaaatcagctcattttttanccaataggccgaaatcggcaaaatcccttataaatnaaaagnntagaccgngatagggttgatgttgtttccagtttgggaacaagagtccactattaaagaacgtgggactccaacgtcaaaggggcnnaaaaaccgtntnatcaggcgatggccccactacgtgaaaccgtcaccctaanccaagttttttgggggtcgaagggtgnccggnaaaagcacttaaatcggggaaaccctaaaagggggaggcccccggatttttagagcttggacgggggggaaagnccgggnggaacgttgggnggaaaaaagggnaagggnanaaanccngnnaaaggnaggggggnctnnaggggcgngggaanagnagggggggnnngggggggggngagnagcgagnaaagacncggggggnannganagggggggg

SEQ ID NO: 52 - DNA, 951 bp, Rattus norvegicus
aaggtcgaaattaaccctcactaaagggaacaaaagctggagctccaccgcggtggcggccgctctagaactagtggatcccccgggctgcaggcacatatctaagttgcccaaagcaccttagaagcagaggctacacagcttttctctgctatccattttccttacccttcctacaccacctctacagccaaagaagggggaggtgggtgcttgtagccccagccccacttagcactgatgtcctacccctccccagcactgagcaggcaagtgctccaagacctcttcctagggacagccagcctggctggcacatttccccaacaaatgctccctggccacacggggcagctctcaccacctccgggctggccaaacagcagtctgcgagtcagtaagtagtccgaggctagcagtctcccagccagctctcccgggatgctcctgccagcacagggttcagcagggcatgcatgccccaggcagagagaatgagccatgctgccctttcctgctcagggncccttgtcctttgggttaagtgtaagacgggggtggtgaaggctccacattgtcagtgctcaggaatgtgaactgggagaacgctgaagccataatccccaactatttcccttggctggatgcccaagtaatcagctgggccaatctacagccagactccagccctgctgcttcaaatgtgggaagtttagagaagaggccatgaagaatctgaatggattgcacagttactcctgtgggttcatcttaactgggaaanantttgttctgtagatataataaatattaacctagnattgggaaaaaaaaaaaanaaaaaaaacccnngggggggggccnggnanccnaatttggcccnaaaaaggggggnnggnnttaaaattcnatngggngggggttttaaaaaggnngggaatnggggaaaac

SEQ ID NO: 53 - DNA, 962 bp, Rattus norvegicus
gcaagntcgaaattaaccctcactaaagggaacaaaagctggagctccaccgcggtggcggccgctctagaactagtggatcccccgggctgcaggaattcggcacgagattatactataaaggttttcagtggatcaaaatgttctactatggaatatatggggagccagccttaatcataaataggagttaactgagggtggacaagagcaggctattcttacaaatgttctgcataaaatgatgcattatataattaagaaaagggtatttcatttttcttatgtgtatgggtgttttgtatgcatgtgtgtctctgtactgtgtgtgtgcagcaccctctgagttaagagaagggaatttggaactagggatgcagatggttgtgnagatgctatgtgggtgtagggaatagaagtcaggccctctagaagagcaaccagtgttcttaactgctgagccatcgccctatcccaatatttataattttaatttttttggaaacaccgcctcagttatcctaggctggccttggaatatgttctgtagctgagcatggccttggaacttctcatcctcctaactccagcctccctgccggattacaggtgagtgctatcatgctcagtttatggcatgttgacatttaaggccctggttcactaattttatgcaaagatctacatctctagccccatacatatattatttaagggggttanttttataatgggatatanggtanatgggccttagcattccnatcaaaaaataaaatgggatttangaaaaataggaatataggcaggaaacntncntttttggnttggccngaagggatggaaatncatgggncctggnaaccanacntaagggcaaaccattaaggncacctgaggntaanggagcccccaggtncccaaaggaanttttgggggaccnggaggccctaaccgggaag

SEQ ID NO: 54 - DNA, 991 bp, Rattus norvegicus
ncaaggtcgaaatanccctcactaaagggaacaaaagctggagctccaccgcggtggcggccgctctagaactagtggatcccccgggctgcaggcaaagtggaattcaagttatgtctattatagatgctaaactgaagcacctactgatctctacctacatcagattaatgctctgtgtcccaaatgttcgtcagtgttgtgtgacggtgtttagaaattggcctatcatatcagtaccttcaggcatgtgatataaaaccgctatgtgatgttacttatggtatttaatgaactgctcactctaccttttatacgtgaaactagttcatcagcgtggtacaaaatttaatattttatcaaaactatcattctggccaaatattgttaaactaattttaaagggcggaatgcattagcatttactgcaggtgagcaaaaaaaatttattttggcttttctgggaaatcaaaaggtcatgctgtcttgccagccgtgagtaccccaaatgtcaatataattaatagataattganataaaaatttcgtcaactgggcatctgtaattcagctccatatacaacttcgtcctttccaaccctggtgtacagggttgtgccccttcananttgggntgtacgttccaccatatagttaggtttgtattnacctaaaacaaacttngttanagctgggtggaaancagcccatcggaactagccccatccaatggtcacggtattttagattccttaatcnaacgnncaaaacnagnggtcagttccacaaancttangnggaanaaaatngggccaagggaaaagacnattgaagnaaaaagccgctttanaggccaaggggggtgggcgncccgtaacccggggggncctgggggngccggagncnccgaaaccaccgaggtcaccnggccgnattnaaaccnaacggnttcccagggagccaggggctggcttccaaggcggtgnacntc

SEQ ID NO: 55 - DNA, 956 bp, Rattus norvegicus
aaggccgaaattaaccctcactaaagggaacaaaagctggagctccaccgcggtggcggccgctctagaactagtggatcccccgggctgcaggcacagccaagtggctatgacaacctgaccttcctcccagacaacaaggccaagtggtcacccacctccaaccggaagccagagccgggccctgagcctgtccagccgcccctccggcctcctagtcccatgtcttccagtcccacgccccccagctccatgcctcctagccctcagcccaaagcttccgggtctcccaagacagtccaggcaggggacagtccttcagccgtgagtctatcctggactaaggagcggcggccggnaggggagggcggctacaagggctgtgtggttcgggcaaggacatcgggggcagaggctgatgtggtggttctcaacgaacccaccgccgacgtgggacagcgccagtgcctcgggaagtgaggggagcgatgatgatgatgaccctgaccagaagaagagtctccgccttggcgcagtcgcagacaacacttacgtctagctcagcgcccggactctccgccccaagccactcccctttcctcctctaattaaatagcacttttcgaaaaaaaaaaaaaaaaaacttcgaaggggggcccggtacccaattcgccctatagtgagtcgtattaaaatttcaatggccgtcgttttaaaaagtcgtgactgggaaaaacctgggggttanccaacttaatcggcttgnagcaaatcccccttttggnanntggggtaatagngaagaaggcccgnaacggattggnncttcccaaaaatttggggcagnttgaaattggggaatgggaaattgtnaagcggttaaaaattttgggtaaaaattcgggggttaaaatttttggtnaaaatcaaggncaatttttttaaaccnaaagang

SEQ ID NO: 56 - DNA, 969 bp, Rattus norvegicus
aagctcgaaattaaccctcactaaagggaacaaaagctggagctccaccgcggtggcggccgctctagaactagtggatcccccgggctgcaggcccctggagaggtgaatagatatgaactcagggaactgggaaggcctggtgtcctagggtattggaggcaggtactagatgtgattgctgaaagtccccggggcagagtgtcctttcagcgtaaggataaacacacacacacacacacacacacacacacacacacatgtgcaccccctgattatttatgaatcgaaatatttgtgacttaaaatttttaatgcaaaaaaaaaaaaaaaaaaaaaaaaaaaaaaaaaaaaaaaaaaaaaaaaacncgggggggggcccggtacccaattcgccctatagtgggtcgttttanaattcactggcngtcgttttanaacgtcgtgactgggaaaaaccctggggttacccaanttaatcgccttgnagnaaatccccctttngccagctggggtaatagcgaaaaggcccgcaccgttggcccttcccaaaagttgggcncctgnaatggngaatggcaaattntaagngtnaatattttgttaaaattcgngttaaattttngtnaaancagccccattttttaaccaatagggcnggaaatngggnaaaatcccttntaaatccaaaagantagcccgngnaangggttgantgttgttcccgttttgggaacaagnggnccnctattnaangaacggngggactcccaacggtcaaaagggggggaaaaaaccgtctattcaggggggagggcccnncnnggggnanccattnacancannatcaaagnttttttngggggncgagggtnccggaaaaggnncttaaatttgggaaccccnaaagggggncccccggattttagagnnttngacngggggaaacccggggaaacnttggngatg

SEQ ID NO: 57 - DNA, 888 bp, Rattus norvegicus
aagcncgaaattaaccctcactaaagggaacaaaagctggagcnccaccgcggtggcggccgctctagaacnagtggatcccccgggctgcaggggggcgacgaggtgtggctggccgtcaacgactacaacggcatggtgggcactgagggctctgacagcgtcttctctggtttcctactgtttcctgactagaatggcaggctgggtccagcacccggacgcccgcctcgctccctctgctttccccatcctcactcagacctcttccttcaggaagtccaccctggttcctgacccatcagccctctgtctcctcagagtttctctgggaatcactgactggttccattccagtggncagtttatcgagacctttatgagactatttttttttcaggtgggaagagagaaaaataaatagatcactaaataaaaaaaaaaaaaaaaaaaacncgagggggggcccggtacccaattcgccctatagtgagtcgtattacaattcacnggccgtcgttttacaacgtcgtgactgggaaaaccctggcgttacccaacttaatcgccttgcagcanatncccctttcgccagctggcgtaaatagcgcaagaggcccgnaccgatcgaccttcccaacagttgcgcagctgnaatgncgaatggcaaattgtaagcgttaatattttgtnaaaattcgcgttaaaatttttgttaaatccagcccaattttttaacccaatagggcggaaaatcggcaaaaatnccntaataaaatccaaaaggaatagaccggngaataaggggtttnaagtggtngntnccaagtttgggaaanaagaaggcccancgaatttaaaggaacggtggganctcccaanggtcaa

SEQ ID NO: 58 - DNA, 931 bp, Rattus norvegicus
tagcgcaagcncgaaattaaccctcactaaagggaacaaaagctggagctccaccgcggtggcggccgctctagaactagtggatcccccgggctgcaggcttcaatcccaacctttacaatgatggcaaggtttgtttaagcatcctgaatacgtggcatggaagaccagaagagaagtggaatcctcagacatcaagttttttgcaagtgttggtttctgtccagtcccttatattagtagctgagccttacttcaatgaaccaggatatgaacggtctagaggcactcccagtggcacacagagctctcgagggggggcccggtacccaattcgccctatagtgagtcgtattacaattcactgggccgtcgttttacaacgtcgtggactgggcaaaaccctggcgttacccaacttaatcgccttgcagcacatccccctttcgccagctggcgtaatagcgaagagggcccgcaccgatcggcccttcccaacagttgcgcanctgaatggcgaatggcaaattgtaagcgttaatattttgttaaaattcgcgttaaatttttgttaaatcagctcattttttaaccaatagggccgaaatcggcaaaatcccttnataaatcaaaagaatagaccggagatagggttggagtgttgtttccagtttggaacaagattccactantaaaagaacgtggganttccaaacgtccaaaggggcgnaaaaaaccgtcctatcaggggcgnatgggccccactaacggtggaaaccatcaacccttaaatccaagnttttttttggggggtaaagggtgcccggtaaaaagccacccnaaaatcgggggaaccccctaaaaggggaagcccccccgggattttaagaacccttggaacgggggggaaaagcccgggcgaaacggtggg

SEQ ID NO: 59 - DNA, 964 bp, Rattus norvegicus
nnaagcncgaaattaaccctcactaaagggaacaaaagctggagctccaccgcggtggcggccgctctagaactagtggatcccccgggctgcaggtgagctcttgctgcagcttctctcctctgccctggtttctgcctgacattagaaagcagcccaggagaaaatcgactccccggacgctgatttcctgtgtcaccttttgatgagtgttcctgggctctgccattggttttcgcctccctgcgacacacacaggaatggccatctccagggtgtggcggaaccgcctgtccttcatggccatcatgatcctcgtggccatggtcctgtccctgcatgtcctacgctctcctctggaaggctggacaacctccactgacgtacctaacctgcagaatcggcttctcacaacttnctgtctgtggcaaggcaggcacattggcttctctnagagtgttacaactttcctggagctgggggtgctggggatacctcaagttggccttgccctggctagggcttggtgtgtatggagccctggtcctcgcatcttcgtccctctgnctctcctccttgcccagtgcaacagtggatgcagggcacaatggcgggctagccgtgggctntnctgggggcaatccnctgatgctggttgngacggngggaactaaagncctcttgcnctnttccttgggnggtggaaangggctccangntcctcncttttcctggggggncctgngcctttnctaancnnctngtggacnctnggccccaaggaccnttaactccaaancaatcntnnggcctnaatgggcctaaggggtnaaanggntccccaancnaaccgaagganaaaagaaaggggancaanggaaaaccaactgggggnaacaaactggcctnaagncccaaggncccntnaacaaaaaagggccctaanngnggaaanggggaaatg

SEQ ID NO: 60 - DNA, 868 bp, Rattus norvegicus
ggcaagcncgcaaattaaccctcactaaagggaacaaaagctggagctccaccgcggtggcggccgctctagaactagtggatcccccgggctgcagggcctggtgctgaccatctttgctaacctcttcccctcagcctacagcggcgtgaacgagcgcacgttcttggcagtgaagcccgacggcgtgcagcggcggctggtgggcgagatcgtgcgtcgctttgaaaggaagggcttcaagctggtggcactgaagctagtgcaggcctccgaagagctactgcggngagcattatgtcgagctgcgggagagacctttctacagccgattagttaaatacatgggctctggtcccgtggtggccatggtgtggcaagggctggatgtcgtgcgcgcttcgnggnccctcataggggccactgacccaggggacgccacgcccggtacgatccgtggtgatttctgtgtggaggttggcaatgctcagagagagatcgctctttggttccgtgaggatgagcttctgtgctgggaggacagcgcgggacactggctatatgatagacgctaaatcaacattaccaatctggaggttgttggtcttctgtgatcttcacatgaacatgctatgtgggtgcaagtccacccaacccagtctgtccaggggcaaccacttccacatcccaccctctatttcctttcataataaaccgcagaaaacccttttgcgctggtgcagtttcaagacaaaaaaaaaanangnnnannnaannnnnnnnagnngaanngnnnnnnnnnnannnnnaaaaaaaacctcgagggggggggcccggnaaccccaaattccgccccaaanaggggntg

SEQ ID NO: 61 - DNA, 887 bp, Rattus norvegicus
tnggcaagcncgaaattaaccctcactaaagggaacaaaagctggagctccaccgcggtggcggccgctctagaactagtggatcccccgggctgcagggagaactagtctcgagttttattttatttttttattttctatttttttgcgatgctctaactgtaaagtagactgaagacaaaaggaaaaacacaacaagacacagttcttcgagcagcaaccaacagagagtcagagtcacaggagaacgcctcacgcagccgcgggttaccagggttgtgcaagcatctcccagcatccttgtgctgctgctttaggctcaaccagtctcgccccgggcgttcacgttctacactgtaagaattggacgctccgtgcatccgtataaacgtgcaaggtttgctttgcttgggtggacagcagcccctgtaccatttgaactcattttgtaacagcaatttcgcttgcaaaaaaaaaaaaaaaaaaaaaaaaaaaaaaaaactcgagggggggcccggtacccaattcgccctatagtgagtcgtattacaattcactggccgtcgttttacaacgtcgtgactgggaaaaccctggngttacccaacttaatcgccttgcagcanatccccctttcgccagctgggttaatagcgaagaggcccgcaccggatcggcccttcccaaaagttgngcagcctggaatggcgnaatggcaaattgtaagnggttaatattttgttaaaattcgcgttaaatttttggttaaatcagccncattttttaaccaatagggccgaaatcgggcaaaaatcccttaataantncaaaaaggattganccggnggattaggggttgaantggtggttnccagntttnggaaana

SEQ ID NO: 62 - DNA, 864 bp, Rattus norvegicus
tnccgcaagcncngaaattaaccctcactaaagggaacaaaagctggagctccaccgcggtggcggccgctctagaactagtggatcccccgggctgcaggaattcggcacgagccaagacgatcttttcctttaggttgaatatttgaatcttatgtgtatcaaaaaagaaatgggttttagtactttctgtgccctgatattttgtatactcctgacttccccagtgtgctggctctgagggcgtgtggagagctctgtaatgcctggttgggcactgctgaggggcctgccgagcttgtttctatttcatactttttatactttgtggaaaaagtcaacggaaaactatagtattggagggaaccagtgtgaccaaggnaaaagatgatttcaacaagcagcctccatgggnacttggcgtgcactctgggttccagttatctcgagctgctccacccctccccagcccaacggttctctctgcaaacgcttggatctaagaagctagtctcctgggttagctgatgcctgccctgctttctggttacttacattctgtttcttgctttaaaagaaagacaagactgttggaccagtattgcaattctgtagagtcgtttcttattaaaacaataatgtgattaccaaaattggcatatttaaggcctaatgccattctaataaaggcaaaaatttctttttacnactaaaaaaagaaaananaaaananaannaaaaaaaaaacccgagggggggcccggtacccaattngcccnatagggagncgtattacaaattcactgggccggncgttttaaaaaangtcggtnactgggggaaaaccctngggggttacccaaa

SEQ ID NO: 63 - DNA, 864 bp, Rattus norvegicus
cncaagcncgaaattaaccctcactaaagggaacaaaagctggagctccaccgcggtggcggccgctctagaactagtggatcccccgggctgcagggggggggtgttatgtgtacagtggaatgaagaccagaagagggcattggttacagggagttgagatccaccatgcaggggctgcaaatccttttgtaagagttcttagagcatatttttgttgttgttggttttttgtttgttgtttttttttttgtggaaacagggttttcctctgtatatctggctatttgaactcagatctatctgcctctacctcatgagtgctggggttaaagacctgtgccaccatactgagctctgtagtaacagctcgtaaccttggaaccattggcttaagtctgggnaaacncctaatagtggttatttctaagacctggaacttggaatcattagttttggtgggtatttttcagttgagtggaatgaatcactcaaattactgaagttataatcttccaattaaaaaaaaaaacatctgcgggttggggatttagctcagtggtagagcgcttgcctaggaagcgcaaggccctgggttcggtccccagctccgaaaaaaagaacccaaaaaaaaaaaaaagggggggcccggtacccaattcgccctaaaagtgagncgtattacaattcacnggccgtcgttttancaacgtcgtgactggggaaaaccctggcgttacccaacttaatcgccttgcagcaacatccccccctttcgccaactggcgtaatagcgaagaggcccgcnaccgantggccctttccccaaaccaagttggcgcaagcctggaaatggcggaaang

SEQ ID NO: 64 - DNA, 899 bp, Rattus norvegicus
caagctcgaaattaaccctcactaaagggaacaaaagctggagctccaccgcggtggcggccgctctagaactagtggatcccccgggctgcaggaattcggcacgagagtgtaaccgctgtctgcctggttgaacttctgggatcaagaaggtgtgttgaaatcggtttcctttgggagcggtgggcacagctaacgcaactgtgaacagacacgtctcacacaatcacctgctgctggcactcggcctgggtctgcctttgcccgccctgccctccgccatagctgtgtggtggcccttagaatagatggggaggcttcaggtagcagccgtgggactgaccaccgctgggcttggggcgctttggctgcacccctgctttcttaagtcttaagtgattgccccatccaagccatggtccccactcctccactcccacccttgggccaaagcttagattgtaatctcccttccctctggaaattggccgtgggtgaggaattcagggcttcccgtctccccacctttatcaaggggtgctgctttcccctcctcaagtcccttgttgcccgtcaccacccaacacttgctgtggccagaagccaccagatgaggttggaagagcctggcctccctcaattagctccggaccacaatcgttcacctgccaacagcctgggaagggagcgccgggtcctcgggccctgccaacaaccatcagcccttgagctttgagctcaggtctagaggtgaacagagcagtcaacgggggcgaatcaagaaggggccaancgntcaaggggtcccttgggaatataantgccttagaagaaaagggccaatgcnggagaagntccttcgggtggnanaatggggtnctgnagtttgggttcctttg

SEQ ID NO: 65 - DNA, 941 bp, Rattus norvegicus
gcaagcgcgaaattaaccctcactaaagggaacaaaagctggagctccaccgcggtggcggccgctctagaactagtggatcccccgggctgcaggcactttcctaaatagaaaanggtagctcacaggcggcagagcacagaaacactggtgggtgtgcccagccagatgccagagtttctgtgctctgccgcctgtgagctaccactttcctaaatagaaaatggcattatttttatttactttttgtaaagtgatttccagtcttctgttggcgttcagggtggccctgtttctgcactgtgtacagtaatagatgcacacggttgacctgtcctggggcctaggtgggttgtacactgagcatcagctcacgtaatggcattgcctgtaacgatgctaataaaacgtctccttcttaaaaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgnagcacatccccctttcgccagctggggtaatagcgaagaggcccgcaacgatcgccctttcccaaacagtttgcggcanctggaatgggcgnaatgggcnaattgtaagcggtttaaataaatttggttaaaaattcgcgttaaaaattttggtaaaatccagccncaatttttttaaacccaananggccggaaaatcgggcaaaaatnccccttataaaatccaaaaaggnattagacccggngaataaaggggttnaaatggttggntncccagttttgggaacaaagaggtcccacctaattaaaagaaancggngggaccncccaaanggtcaaaaagggggggaaaaaanccgggctatcaanggggggnanggc

SEQ ID NO: 66 - DNA, 877 bp, Rattus norvegicus
aagcgcgaaattaaccctcactaaagggaacaaaagctggagcnccaccgcggtggcggccgctctagaacnagtggatcccccgggctgcagggtctgagctccctaccctgnaagaggctgtgggcttgctcgtagcctaacccctcaccgacagtttgatggcgaagagacggtccttgtgcgtaaggaaggacagttctcacnccgcagaataacaggagacagaaatgttcaattaaaaagagtttactttagaccacagcctgctgtgtgcccactcactgccctgtctgctgggaggcgggatcaggggagataggcggatggctgtctcattaatgtgctatgcctagttagtgtggaggtgggagaaaggaggtgtgtgtgtgggggggggcgggggatcttgttatgtaaccctgtgctttctgtccttgaaccnctggggtgggaggaaccctattatctgcctctcggataacaaaggacggattgattattctggggacncctaagtggggagaggggtgaggcatttgcaagtgacccctgggacctggaaccctcaagaggagcctaatgtctctgcccacaggaacatctgtggnttcantacttcttgttttgtccctgtatgcttttctctgcactcaaatggggtgatgagggaaggcggggggctctcaaatcctgtctgtgaacttttcccttcttgctgatgcnacttcttcgncaagccaaggtctgaaagaaggagtcctcccaggntgntgncngnttgccggagatcataaagcananggtcaggggctggncnccctgggnnncaagtncgggctttggccngtgggatgccnggggcaaaaggacctng

SEQ ID NO: 67 - DNA, 895 bp, Rattus norvegicus
caagcgcgaaatnaaccctcactaaagggaacaaaagctggagctccaccgcggtggcggnccgctctagaactagtggatcccccgggctgcaggtgggacgccggattcgcaagcccgatttggtcagtcggtgaaggggcttctcacggaaaaggtgaacacctgcggcaccgacgtaatcgcgctcaccaagcaggtgctgaaaggctcgcgaacttctgagctgctggggcaggcagctcgaaatatggtgctacaggaagatgccatcttgcactcagaagatagtttaaggaagatggcgataataacaacacaccttcagtaccagcaagaagctattcagaagaacgttgaacagtcgcctggacctgcaagaccagctggagtcatttactggaagtagctctcgccagaacagcagttggacttctttggcctgatgctgagaaggacgcgggccttagtttccattttcatctccaaaaattcatctagaaaagacttgtgaaaagaagaagccaagtgaccaaacgtgaaagcacttcttaagttgggagtaactcatcttcaagtggttttatattaaaattaataggttgaatcatttagcatcagatccctctctctctctccctcctgtgtagcccgctgcttttgaactcataatccccgtgccgtagcctctcaagtagtgagattaaaaaccaccatacctggccactggagtaacacattagtcgcagatacttgggaggctgagggcagggaggatcctgtggagccccnaacggttgagtccatccagggnnacacaggaagaacctatctccaagaaggagaaggggcaaggggaagggggcttctaactggggatcacgtgagaagtgnt

SEQ ID NO: 68 - DNA, 947 bp, Rattus norvegicus
tntgcaagcncgaaattaaccctcactaaagggaacaaaagctggagctccaccgcggtggcggccgctctagaactagtggatcccccgggctgcaggaattcggcacgagctactttccagtgtaaccatagcctgcttagcttggggtaagacttagtagaaaatggtgcttcagtaaaccgcttacttccagtcacaatcaccttgttgctgtgggacccgaccctgtttgagccggctgccctcattcccacttaaatcaaagcagctggctagtccccctgtttccttcccaaatcctgttttcatgtacaagacaaaataaaggactcaattctccctacaaaaaaaaaaaaaaaaaaactcgagggggggcccggtacccaattcgccctatagtgagtcgtattacaattcactggccgtcgttttacaacgtcgtgactggggaaaaccctggcgttacccaacttaatcgccttggcagcacatccccctttcgccagctggcgtaatagcgaagaaggcccgcaccggatngcccttcccaacagttgcgcanctgnaatgggcgaatgggcaaattgtaagcgttaanattttgttaaaattcgcgttaaatttttgntaaatcagctcattttttaancaatnggccgaaatcggcaaaatccctttataaaatcnaaaagantaggaccgagatagggtttgagtgntgttccnagttttgggaacaaagangtnacacctatttnaaaagaaacgtgggagctcncaacggtnaaaaggggcggaaaaaanccggtctaatncangggcggatngggcccaacggaanggtgaaacccaatggaanngtaaatcnaaggattnttnnggggggncncgaaggtngccgggaaaaggcacctaaaaatncggggancccntaaaagggggng

SEQ ID NO: 69 - DNA, 895 bp, Rattus norvegicus
caagcgcgaaattaaccctcactaaagggaacaaaagctggagctccaccgcggtggcggccgctctagaactagtggatcccccgggctgcaggtttttttttaaggatggaaactgtttatttatgggtaaagaatagctgagagaacacttgaatttgatgaagctgtgccactttgcaggtcgggttggtttatcatcaaaaggtccaaaataaaagttacttcacagaaaggaggaggaagcaataagttaatgcataataagcgcttttacaagcatactttataggaaggagattcataattatagccaatatattctagacagtaactttgactatttcacaagaacataaaattactgagtatggaatgggtggcagacacgaccatggacgaagaagggcatatgttgtgtacctggccatggatcacagctcctaagctttggaactacattttggctgtgggacacaagaacataagattttctctaggagttaagggagtggccaatgggctgatagtgggcagtggagaaagaacactgtacattcttaaaagtctgccatagttgaagagatgagtgaggtttgtagttaacaaaaacatggactttttcctttttaatacaggtttacctgctaatgcaaatttagaaggaatttaaccaagtcagtaaaaatgttgaaggctttcaccggaaccaatgactgttttggcctctttattcaaagtacaagatggatgtcaccaaaactgggatttgagantggaaaatttccaaaagggggagaaaaatccnggggttatttanaggttaaaaaaccggggaaggatttggttaaaggccancaggataggtncaggacccaattgggaacccatatnc

SEQ ID NO: 70 - DNA, 896 bp, Rattus norvegicus
ncaagctcgaaattaaccctcactaaagggaacaaaagctggagctccaccgcggtggcggccgctctagaactagtggatcccccgggctgcaggaattcggcacgagaagaactcaagagagtagccaccatcttaaagcaaagtagcaggtggggaaaaggtgggtagaggagatgctacttagggggtgggattttccagtcagggccatttagacacgggaatcgctgaggctttcagtcgatggggctttcttttttccctgcttcatctctcggcctcaggagaggtattaacagtattatcaccatttatatcctagctgtcctgagccaaatctgccattggaggtgtcttccctgtgttggttctccaagggacgttgcatggggatttgtgggggcagggcagcaaggccttgttctctaaatgtccagaaacactctaattaccatttccacctgggtgcctcacacactccccagaggggaggttaacatctctgccccatttccctcatgttccttggcttggtcattccctacctttctattttgtgttaaacttggcttttttttttttttcatattgaaaagatgacattgccccgagagccaaaaataaatggggaatggaaaaaaaaaaaaaannnanaannnananannnannannaaaaaaaancccgngggggggcccggtacccaattngcccnaaaagggggnggnatnaaaattccngnggccggncggttttaaaanggncggnangggnaaaancccnggggggttnacccannttaaancgccttnggaggaaaaatccccccttttnngcaaaannggggggaaanaanggaaaaagggcccggaacnggnatggcncttttccnag

SEQ ID NO: 71 - DNA, 929 bp, Rattus norvegicus
cncaagcgcgaaatnaaccctcactaaagggaacaaaagctggagctccaccgcggtggcggccgctctagaactagtggatcccccgggctgcaggaattcggcacgagaatagagcttttgtgcggcggcagcggcggcggcgtctctctgatttgaacgccgaacagcggtagcttctcatctgtggcctgacctcgaagcctaagaacagagcggcgagatgacggaccggtacaccatccacagccagctcgagcatctgcagtccaagtacatcggcacgggccacgccgacaccaccaagtgggaatggcttgtgaaccagcatcgggattcctactgctcctacatgggtcacttcgacctcctcaactnacttcgccattgctgagaatgnagagcaaagcgcgcgtgncgttttcaacctggatggagaaaatgctgcagcccagcgggccgaccggcgggacaagccggaggagaactgaggcgagcgcttcccagccttccccatctgccatctgtggaccgatcctcctgactcctgcttctcgaccattctccgttgggtgtatcgcctgacctggcttacctgtgggacggttccgaacaagtcatcgagagactgtcgggtctcctggggaagctgtgcgggaaggagtgatcccagaatcgggcaaagcgangggagaagactgcctggggaatggatgacgcattccgagttcagcttttcgaataagttgatgtcgttctcgccttttttntttttttaaatanntannatacataaaagttagggattttgntaaaaaaaaaaaananaaaaaaaaaacttcgnagggggggggnccgggtaccccaaatttcggcccctaaanaaggnggaagtncggnatttaaaaaatttcaacttgggccngntg

SEQ ID NO: 72 - DNA, 944 bp, Rattus norvegicus
ntcaagcgtcgaaattaaccctcacntaaagggaacaaaagctggagctccaccgcggtggcggccgctctagaactagtggatcccccgggctgcaggcggccttggaaccgctcgatttctatagagaagctggcggctggtgccagtggcttctgagttctcgattcttggacnccgagaaggctgcaaggccatgctggcttggcgcgtggcacgcggcgcgtggagggtcccttcgcgtggctgtccggcctccgggggcgcggctcggcaggggcggctcccgcaggccctgctaccacccgcggcctgctgcctgggctgcctggccgagcgctggcggctgcgtccggccgcgttcgccttgcggctgccaggcaccagcccgcggacccactgnctccggcgccgggaaggcagccccggagcccgcagccggaggagatnccgccggcgcagncccctangnncccggtgggtccgggcgagcgcaaccanctcgtatgaaaatccatggacaatcccaaatttgttgtcaatgacaagaattancctggccccantgttgggctatctgattcttgaagaanattttaatgttgcacnagggtgtttttgctttaactaggactaacgggatttgttggnatgggnatttaatnncnnnaaaactnnggcncaatnaaaaatnaagctttngggaaantgctcttggatcccaactngcttgnanaaangtttnttaancaaggaatcttgaanaagaatnangccnggnanctaatgnnnagaatccttaaattcgaaantncccaagnnaacttaacaggnntaaattttcaangaagnatggaaangggtggaaacggcaggcngagnnntnttaannnnnaanataaacgagaaannnntggngaaaaaaccggggaaacagantanng

SEQ ID NO: 73 - DNA, 886 bp, Rattus norvegicus
caagcgtcgaaattaaccctcactaaagggaacaaaagctggagctccaccgcggtggcgggccgctctagaactagtggatcccccgggctgcaggccatcgccccactccaccctccttcctcttgggcttggtgtggatggacctgtccatcagcttggtgtggtgttcacaaataactgacaggccagggaaggtcctgctgtggccgcctgagagatggctcaagtactctgtgattccttgtggcccagagccctgtgtgctggctgctatcagacaccttgcccctgtgctggtcttaaagaatcattatccatcgtggtcactacgtcctgcttcctctgggcatctggagttcccagattcctctgtccctttcctggatatgcttttgtatacacatttttagcacgagactttccccctttacaaaggtgtcaacttgaaaaatgttttaaaccacaggatagcactttcaatctaacttttgtggtatcttccatcagaatcatcttttcctatctgttttttcccttcacgggttaaggttcacatccccatggaaaccagtttaaatctgcctcagaacaatttgtatctttgggaaggaattgtgtccttggaggcccatgtaagtggattctaagtgggggccagctgctcctgtgtgcatgctgggactctggggaggagagccccctggcatacacctcaatttgccctcagtcaaggtgaggcaaggggtgctggagtttcctcancccagcaaggctttgctgtcaataaggaaggagggaagaaaagtcncccggtggcnaatgaggaccatagcatacctaananggcccaataagaanggaaacaagtggnctacnaaagccg

SEQ ID NO: 74 - DNA, 888 bp, Rattus norvegicus
gcaagcncgaaattaaccctcactaaagggaacaaaagctggagctccaccgcggtggcggccgctctagaactagtggatcccccgggctgcaggcatgggcagagttttcaccaccaaaaacatgtgcctcaagccagtacccggatccctgaggccacagaagggaaacctccagacacaagcacggctgtacagtttcagagcacccagcagtccacttttccatctggagcaccatccttgaacaaagagctcacccgccactgggaaacaaccattctcccttcaggctatggtctggaggctaggcctgtggctgaggcaaatgagaaacagcacaaacagcaaaaagaaccaggagctggtgctgggcacacaagccttggtgccggtgctatccctcctgggccatcgtcttcttcctcgtgggcagccatggtctgtgtgctgtgcaacaggaggagtgactcgggcagagctcttcaggtttgcagctctgttatacacctgttgaaccaagaccccagggcctgtagcaagaatggaagctcgtgagagatgacgtgggagaggtggcagacagactggcaggctaggctccatcaacgaactgaatctgagctcattttttctggtgatgttttgaatcaaagtagccattatgtaatctagggacagctttgaactacagagcctgtgccccgacctcctagattctgggattatagatatatcctactgcatctgccctcgtctaatttcataaataaggtntaaattttcanggttttgttttggtttagcggaagaatcttatttagnccaagccaacctcaaaatnccccaagggaancnctaanggnccccaggccttccttaaaaaag

SEQ ID NO: 75 - DNA, 893 bp, Rattus norvegicus
caagcgcgaaattaaccctcactaaagggaacaaaagctggagctccaccgcggtggcggccgctctagaacnagtggatcccccgggctgcagggagaagagatcctggatcacagtgctgtccgccatgacagaggaggcagctgttgcaatcaaggccatggcaaaataactggcttccagggtggcggtggtggcancagtgatccatgagcctacagaggcccctcccccagctctggctgggcccttggctggactcctatccaatttatttgacgttttattttggttttcctcacnccttcaaactgtcggggagaccctgacccttcaccnagctcccttgggccaggcatgaaggggagccatggccttggtgcaagctacctgnccttcttctctcgcagccctgaatgggggaaagggagtgggtactgcctgtggtttaggttcccctctccctttttctttttaattcaatttggaatcagaaagctgtggattcnggcaaatggtcttgtgtccnttatnccactcaaacccatctggncccctgtnctccatagtccttcanncccaagcaccantgtacagactgggggaccagcccccttcccngcctgtgtctcttcccaaacncctctataggggtgacaagaagaggggggggangggacacgatccctcctcaaggcatctggggaangccttgcccccatggggctttaaacctttcctgtggggtttctccctgaaaaaatttggnaaaaatcaaaacctgnataaaacganaagnttaaatatggaaaaaaaannnnnnnnnnnangnnnnnnnggnnnnantnnannnngnanaannnnnnnnnnnanggggnngggggggggnnnn

SEQ ID NO: 76 - DNA, 940 bp, Rattus norvegicus
nancgcaagcgcngaaattaaccctcactaaagggaacaaaagctggagctccaccgcggtggcggccgctctagaactagtggatcccccgggctgcaggaattcggcacgagccgcatccaagaagacaggtggcagttctaagaaccttggtggcaaatcacgaggcaaacactatggcatcaagaaaatggaaggtcactacgttcatgccggcaacatccttggcactcagcggcagttcagatggcacccaggcgcccatgtgggactggggaagaacaagtgcctgtatgccctggaggaggggatagttcgctacacgaaagaagtctacgtgcccaatcccaaaaactcggaggctgtgaatctggtcactagtctgcccaagggtgctgtgctctacaagacttttgtccacgtggttcctgccaaaccggaggnaaccttcaaactggtagacatggctttgaagtcctgttgagaccatcggatgacgggcgaccggaacccaggtcacaggagcaagtgatgatggaagtcaagggtcagggtgaggacaaggtctccacagaagaggcctattggatggggactctgcaggggcctttgtgctgtggttgctggaaanctcttggnagctctggcatgantgtcaataaagctgnaggaattcctggaaaaaaaanaaaaannaaanaaaaaacctcgagggggggggcccgggttaccccaaattcgnccctaatannngaaancggnaanttaacaaattcaacngggccggtcngntttaaacaaaggtccgtnganctggggnaaaaaccccngggnnggtttaccccaaacttaaatccggnncttggagagggaaaattcccccccttttgggccaaggcctgggggnaagataagcgganaaaagggccccgcaacccg

SEQ ID NO: 77 - DNA, 896 bp, Rattus norvegicus
cgcaagcncgaaattaaccctcactaaagggaacaaaagctggagctccaccgcggtggcggccgctctagaactagtggatcccccgggctgcaggtgttagcctccggtaccggctctcttctttttcaatgtagcgcttgaagccctagctagagcaataaggtaaggaaactaaaggagcataaatagaaaaagtcaaattatccctattagccaatgatattatgcatatgagattctaaacactttcaggagagtggaaggatacaaaaacaaattacaaaacccagtagctcttctctatgccaataacagcatcctgcgaaagaaatcctggaaaaccatcccattcacaacaacctcaaacacgtatacattcgtactcatccccataaaaagcctaaccaaggggttgcgagacctctacaatgatgattttacatctctgagggaagacaatagaggatgggaagaccttccacgctcatggactggtagaattcagagtgtggcaatggtcacgcctaaaaaccatgtgcggattcatgacagtgcaaccacctggtaatcaaccaccgaaaaatgcaaacgagaggacagaaagtattatttacaaacggggctggaaaacattggatgtccacaggtagaagaatgagattagaacctttaccgcttcctctggcacaaaaccaactcccaccaggtccaagatgccagtgngaaactttcagtctctgggaaccctgcagactcttggagagcaatggttacacagggagaatcctcatttccggctttatggggggagactacctggagggaaaagtgcccngtccttccccnggggcttctaaggaaacccttcttagaggaggggtaaaaattaaacc

SEQ ID NO: 78 - DNA, 892 bp, Rattus norvegicus
caagctcgaaattaaccctcactaaagggaacaaaagctggagctccaccgcggtggcggccgctctagaactagtggatcccccgggctgcaggcccaaccagcccacatttgttctctaaacccaaacagctgtcccctgtctgtctgtggctttttgttcattttatttagtgatgttttttcagttaaaccaccgtggacaaatgtctcactaagaaatccgtgtgaagctgtatagcttacacctgtaattgtagaacgtgggaggctggactgaggggatagcattgagttcaaggccagccagagctgtcagctatatagagttccaggctgatctcagtcacagagtaagaccctttctcagaaagacaaagaatctaaatgaggtaggattctgtgggctcagtggtaattggcctaacttggctgttcacacttgtaaggcccagaatttgatccccacaccctccaaaaaagaagtttggggatgtgaactgaattagcatcagtgcctctgatcctctctcagccgtagactagaatgacgaggagccctggtttaaccttggcactgctgcctaccctctctaagctcgctttcctcatctgtgaggagcctcgggatggagcctcagggagtgcgggtggatatttttatattgtctattaaaatgtaggcattaagctccaacatttntgcttgttacaattttanggcctatattttattgattaaaaaatgcccctggcggggttgggggatttagccccagtggtagagcggcttgnctagcaagcgcaaaggccctnggggnttgggttcccagcncccgnaaaaaaaaaaannannnnnannanaaaaaaaaccnncgagggggggggcccg

SEQ ID NO: 79 - DNA, 979 bp, Rattus norvegicus
aagcgcgaaattaaccctcactaaagggaacaaaagctggagctccaccgcggtggcggccgctctagaactagtggatcccccgggctgcaggctgacatgtggcactggtggtttttcattaccttggatgtcagaggactttcattgaaacaaaattttatgttggactggaaaatgggggctagaagtatcatgtatggtcaaacggaagactgggtaacctccaaacagagcattcatgaaattgtcaacagtattcgtcccaagtattttcatatgctgtcacacaagaggaccagatgctggaggatctccagtctgtcccctctcagcagagaggaaagacagtatggcagaaaacctgtgtagctttagcttcaggtcctgttaaagcattactgtttgcacagcaggaaattccccctgnaactgtcagcttttccctgtgttactggcactgttggaatgaggtggaaagtacacgaatggatgccatggtcttgttggtggtcagggtcctactgccgtgtaatgaaggcctgcagtgcagacactcacttgtttctctctattcacagtattctccggaaacgcattcgagaggatagaaaggctacaaccgctcagaaggtgcagcagatgagacagaggctaaatgaaactgaacggaaaaggaaaaggccaagattgacagacacctaaatgttcatgacttgagactattctgcagctataaattttgaacctttgatgtgcaaagcaagacctgaagcccactccggaaactaaagtgaggcttgctaancctgtagattgcctcacaagntgtctgtttacaaagtaagctttacatccaggggatgaagaaacgccaccagcagagacttgcaaaccccttaaantnganggaattggnnttttaaccanggnggtatgaattggaggaaagatgtaaagnaaaatnaatttagggggg

SEQ ID NO: 80 - DNA, 973 bp, Rattus norvegicus
agncaagcgcgaaattaaccctcactaaagggaacaaaagctggagctccaccgcggtggcggccgctctagaactagtggatcccccgggctgcaggtttttttttttttttcacaatgaatatgtctcatttattaggtagaaaacacttaactgcataaaacttacagggaaaaatggccgtatttgaaaacagctaaaaggatcagagtagaacacagaaccgtaatgagcagtgtcacggagcacactaaggaggtgtgtatagtcagtcccactggcacctgcactgtcaaattcttaagtattgatttgtactgcatgtttttccactgggcagatctcctcactcttcaaagaacaagggagctgctactttctgactgagcccagcatttcaaaattggggaactcttggtcacagtgcatcagtaagtcagggttgttgaccacaatggagggtgtctccatccttcttatgtggacgcaattttggggctccttcgggcacttcgggcacctctacagccaccgcggcaccggcgggcgggtttggcggatttggaacaacaactacaactgcaggctctgcattcagcttttctgccccaacaaacacagggcagtacaggccttctcggcggnactcagaacaaaggttttggctttggcactggttttggcacatcgacgggtactggcactggtttaggcactggcttgggaancggacttggattcggaggatttaacacccagcagcagcagcagcagcagcagacttctttaggcggtctcttcagtcagcctgcacanggccctgcgcagtccaaaccaactcatcaaactggcagggtctttctggnccaaagcnaantggggggtgaaaaaangccancttggcaaagtgggaccagttgcagggcttctgggggaaagggaaaggggatttccataanaaaaaatcccccc

SEQ ID NO: 81 - DNA, 1004 bp, Rattus norvegicus
cgcaagcgnngaaattaaccctcacgtaaagggaacaaaagctggagctccaccgcggtggcggccgctctagaactagtggatcccccgggctgcaggtttgatttcaaatggatctacactgttaactgaatgatgagactccactgtgattcactcgtttacttaatcaaaaaatttcagggatgtctgtaaatttcagtgttgtgcacaacaagaagtgctgttggttgatttaaggagggaccagaaataatttctactattccagtactgaaggaaaaaaaatactgatttatactgtttttaaaaactaaatattaataaagccccctgtcagaaatttaaccttaaaaattattttaaatatcatcctatattattagaagggaactacaagtgactggataaataccaaaaagattcacaagcagcttcatttaaaaagcacaaagaggttctggtgtgaaatgcccaaatctcaaatgttttctgtagttctgagtttacagatgtaaaagctgtccctggaaggctgcagtacctgtatctgctgtccctgtgaactacactcgcaccaccccagaaaccctggtactggatagggtagcgtgggggtacagtctcagagggggtgagccacagtcacccaacggggtcagcttgcagaaagaccaaaacagggaaaggcgagtgggataattacttaacagcacattcactctctacggagtaatcacatatgggttgaaatttgaagagcagattgtggatcattttgacccctggncaaaatccctgtacttggagaantttggaggcggacgtgggcatccacgtgggcggttgcctttaccaacaggnccgtgggactggttgccaaaaaggncggggtncnctggaaaaaaagccaggnccccccncggtggcctggcaaaggaccaggttangggaaaggaaaancccnnnntatntttgnngaacccaaanttttaatnccccgg

SEQ ID NO: 82 - DNA, 1003 bp, Rattus norvegicus
aggcaagcgcngaaattaaccctcactaaagggaacaaaagctggagctccaccgcggtggcggccgctctagaactagtggatcccccgggctgcaggaattcggcacgaggctatccagacgggcagagttacccaggagctgcaggacaggtacctggaccacaccccggtggctactatcctggacctccccatggtgggggccagtatggcagtggattcccccctggtggttacggagctcctgcccctggaggaccctatggctaccccagtgctggaggaaccccctctggaactccaggcggaccatatggcggtggacctccaggaggcccctatggtggtggacctccaggaggcccctatggtcaggcacatccaagtccctatggtacccagccgcctggaccttatggacagggtggtgtcccccccaatgtcgatcctgagggcctactcctggttccagtcagtggatgccgaccacagtggctatatctcacttcaaggagctgaagcaggccctggtcaactccaactggtcctcattcaatgatgagacgtgcctcatgatgataaacatgtttgacaagaccaagactggccgaattgatgtcgtcggcttctcagccttatggaattcctccagcagtggaagaacctcttttcagcagtatgaccgggaccactcgggatccatcagctccacagaagctgcagcaagcgctgtcccagatgggctacaaacctgnagcctcagttcaagcaagcttcctggttttcccgaatactgtaaaaggntctggncaattcccggccaatgcagctgggaatggtttcaatcaagggtggtgtnacccaagctttcaanggtgttgaactgaaggccttccggggaagaaagggtanggggttgtaaaaggggaaaaaattccgggntcnaggcttttgaaggganttttgncaacnatgnaggggtttaanggatngcaatnnn

SEQ ID NO: 83 - DNA, 1004 bp, Rattus norvegicus
cgcaagcgcggaaattaaccctcactaaagggaacaaaagctggagctccaccgcggtggcggccgctctagaactagtggatcccccgggctgcagggtgggcagagggattggccctgctttgtgacccctacctgatgcctccttccacaacatgattgcagcccttagaacctagctcagagcccctcagcacaagccccgcccccagcagccagccaaagccactgggtgagcgggtacatctgcggacccatccctcagcctcgatcaaccaggatccctccttcccagagcctgtccctgggagagcttttgccaccaaggttctagccctggactttctaaccacttcttctcatgggagacaccctggctggctactnccaagggaccagtttggcttaacttcacagcccacatccatggtcatctttaaccttctttcctggcagattgccaggttgctaatctgctgtcccctctggctgtaagcacagtgtcaggacctagtgaggaggtacaaggagcaggccgtgcttggagtgcccattccccctaaccctctgggntggcgcctcctcctcactggagacccggaactctgnagagagccaaagcacacagggacccagcagtgtgggttagacaaagctgcagctaagatcggggagtcctggnactgcaggccaggccagggtccccacctaagccacataatctgcctgccggagnctggcccccgagccccctcctggggaaagtgctgcccatttgccagtgtctgcccaggaggaaggggatctgcttcagagggnttcctgagaaccctgggtcccaagnctnagctggtaaaggcctctggggtgggaaaaggnttgctggtgggggaagnccaaaaacggggaaagttttgnaanaagggnganaaggtttttaagnaggnggagcggaggcaaaaaggggtttttagagccaagggncaanaaattttnttttaatg

SEQ ID NO: 84 - DNA, 982 bp, Rattus norvegicus
aagcgcgaaattaaccctcacgtaaagggaacaaaagctggagctccaccgcggtggcggccgctctagaactagtggatcccccgggctgcaggaatcatcgctgaggccctaacaagggtcatctacaacctgacagagaaggggacgcccccagacatgccagtgttcacggagcagatgcaggtccagcaggagcagatagactcagtcatggactggctcaccaaccagccccgggccgcccaactgctggacaaggacgggacgttcctgagtacactggagcacttcctgagccgctacctgaaggacgtgcggcagcaccacgtgaaggccgacaagcgggaccctggagtttgtcttctatggaccagctgaagcaagtgatgaacgcttacagggtcaagccagccatctttgacctgctgctggccttgtgcattggggcgtacctggggcatggcatacacagccgtccagcacttccatgtgctgtacaagacggtgcagagactgctgctcaaggccaaggcacagtgacagtggccatgcacaggtggcccaggaggtactagcccacgcacccacagcagccagactgaaacacagagggtttggatgggtcactaggatgcagggacacctctccctgtccatttctttgaatgtccctggaggagagccccgcccgcctgcagacgagcccactgggatggaatgatgacccgggccaaatgcactgaaaggccgcacaatgctgttggcctccccagtggctgggttccagaagcctgtctctgcagtttcccaagaggnagccaacgttcagcctggcttggcccagcaacgggcaaganccaanaatgttntccctgatggtcctcctcaaaacccctgttccggccttgtttgttaacttttgnaacaatttgaaccaaccttgggtnccccaagcttgggattttgagccaccctgagg

SEQ ID NO: 85 - DNA, 983 bp, Rattus norvegicus
caagcgnggaaattaaccctcactaaagggaacaaaagctggagctccaccgcggtggcggccgctctagaactagtggatcccccgggctgcagggtttttttttttttttttttaaaacttaaaaacattttttatttttttggtttcgagacaggatctaagtagtgcagattgatcttaaactcagcctgcctctgactttcaagagctgggattaaagatacatgacactatatcaaccaattcaccccaatttatttttcttatatatttaattgtttccttcccacttaaaaaatcataacaaaataatagatttcatgacacttccaaaggaatcattgtactttgctaatatttgctatttgttccagtaatatggagactaaatttagccactcactcctgnaccagtcctaactttaaatgtgtttggattaaacttgtaatcccaggtatttgggaattaggattgaaaagggaggctaggggttggggatttagctcagtggtagagcgcttgcctaggaaacacaaggccctgggttcggtccccagctccgaaaaaaagaaccaaaaaaaaaaaaaaagaaaganaaaaaagaaaatgggaggttacatagtaacttcaaggccaacctagacgggggggcccggtacccaattcgccctatagtgagtcgtattacaattcactggccgtcgttttacaacgtcgtgactgggaaaancctggcgttaccccaacttaaatcgccntggcagcanatccccctttcgccagctggcgtaaatagcgaagnagggcccgcaccgatcggcccttcccaaacagttgcgcancctgaaatggcggaatgggcaaattggaagcggtaaanaatttnggnaaaaattcgnggnaaaattttgggnaaaanncagcccaanttttttaacccaanaggggccggaaanccggggaaaaannccccttaaaa

SEQ ID NO: 86 - DNA, 943 bp, Rattus norvegicus
tcaangcgaaataaccctcactaaagggaacaaaagctggagctccaccgcggtggcggccgctctagaacnagtggatcccccgggctgcaggtttgcatgcccggcaggtgcagacacacgaaggtcttcagctcgccaatgagctgggtagtctcttccttgaaatttctactagtgaaaactacgaagacgtctgcgatgtgtttcaacatctctgcaaagaagtgagcaagctgcacagccttagcggggagcggaggagagcatccatcatcccccggccccgatcccccaacatgcaggacctgaagaggcgcttcaggcaagccctgtcctccaaagcaaaagcagcctccaccctgggctgatccatcgcagacagactgacatagtattatcaataagcatttgtgctgccacaaagactggtcctttcctcctttaaaacatatccaggggttggggatttagctcagtggtagagcgcttgcctagcaagtgcaaggccctgggttcagtccccagctccaaaaaaaaagaaccaaaaaaaaaanaaaaanaacnagaacaaaaaaacaaaaaaccatatccagagtttatttttataatggactttattgggctttcaagtgtatgtatatttctgaaaaattcaaacagtggntttttttaatgggtttttnttttnattttatnttantttacngnaaaccgttagccactcttccattaaaggcaaaaatggcaanaccaaaaaangaaaaganganannnnanananngngannnaaaananaanananaangaanaagaaaaaaancnccnagggggggggnccnagaaacccaattggccnnnaaaagggggggaggattnaaanatncnaggngcngnaggnttttaaaaaggcgnnagccagggggaaancccaggggggtc

SEQ ID NO: 87 - DNA, 939 bp, Rattus norvegicus
cgtcaagcgnngaaatnaaccctcacttaaagggaacaaaagctggagcnccaccgcggtggcggccgctctagaacnagtggatcccccgggctgcaggtgacgcaactttgtcangaaaacgaatgcagtcgctctccctgaataagtaancnggcctgtgggagganatgccgggggaactgggccgtgccgccagganctctgccatgtctcacccactctgtgccctggcgcngctgcagcagcccctacggccangagcccctacggcctggggcctcctcttcatcttggcacagaaattgttcaggggaagnggaaggggctggggggaggggcagctgctatctttgagacagaaagatgcaggcacagcatttcatacgtaaccatttgaatgtttttgactgtttttagaattcgggccctggtggggtgggtggggtgcctgggaatggcgtaaggagattccatttgtccagtagattgcacgttagtgtggggaggggggtgtggtgccagcaggcagctgctgtgggagttgatgacaaccagcccagatcatctgggtgctcactcagaggggctctccgggancctgtgcctcgnaagtccgttccgatgaagcctctcctctccactctgcccccttcccacctacctggtcagggctagtgcccatttttaaccctacccattgancatttcaagaaaacctctggttactgtgctcacccagancaagacgtgctcctcaaatncaacttgnatagntgggcagattaaaacaacattnatncanaaaagaaaannnanaaaggggggggcccggaaacccaaattnggccctaataagngaancnggaataccaaattcaatnggccggacgntnttaaaaaacgnccgnganagggnaaaaccctggncgnaaaccn

SEQ ID NO: 88 - DNA, 1014 bp, Rattus norvegicus
nnctcaagcangnaaatnaaccctcactaaagggaacaaaagctggagctccaccgcggtggcggccgctctagaactagtggatcccccgggctgcaggtcgagttttttttttttttttttttcttttttttaagataatggtttttaattgaattattgagatgaagagacagtgaagccctgtttgctacttacatgaaaagattttaaaaacaatcacngcacaaaatacaaaggggcagggtatgctgtggcattgaatttcncctcactttttttcttgacgtctcaagaacaaattaaagtttccacagcaaatttgttctcaaaangccgaatggtgaaacagttacgggcttcacgcttctgnaataccnctaatggtttccctgacgcngcatttgtaggtttccttgtcgtgacacagtcggnaaatgaagaagcccagggggtccacgttttngangcggtcggtgatcaccatgtgctcatggatgaggtatgacngaggcaagtaggtcccggccttgatgtcaataagaagctccaacagtttcngggnggcataacaanggcaggngtncanaggnatcaagntnncacntganccaanattnaagggcncaaataagnaaangaannntgcangtnnaaanntcatncacaatgnttggncaggaaacgctnnnccgcaaanctccagggnaacaggntanacngnatgcaattacnacgggncgnccatcccacnaaagaagcnaaagaaannctcnnncaaaatagttcaggganancgaannancnnggngagcanccgagaanntaagngcaactnnanacanatattgancgnnncaaccnantgaatgaaaaactganannccnaanaannaggannnnacaancaancacanggnnnnaatgngnngaantaanancaatgaagaaggtgagangnnccgacnccangagaaggaacgnaganacggngntnnanaggggncaagattc

SEQ ID NO: 89 - DNA, 955 bp, Rattus norvegicus
accgcngaaactnaaccctcacntaaagggaacaaaagctggagcnccaccgcggtggcggccgctctagaacnagtggatcccccgggctgcagggctgtgcagcggcggaagttatctctgcagggaagatgcttcccttgtcgctgctgaagacagcccagaatcaccccatgctggtggagctgaagaatggggagacctacaacgggcacctggtgagctgcgacaactggatgaacatcaaccttcgagaagtgatctgcacatcgagggacggtgacaagttctggaggatgcccgagtgctacatccgaggcagcaccatcaagtacctgacgtatcccggcatgcagatcattgcacatggtgagggcaagaggaccgccaagggccgagggcgaggaggaccgncagcagcagaagcagccagaaaggccgaggccatgggtggcgctggcagaggtgtgtttggtggccggggccggggtggcatccctggtgcaggccgaggccagccggnacaagaagccagggcggccaggcaggcaagcagtgcagcagtcccagcctgaactgagtccaggaaggtgggtgaggagacctccgggcgcctttgcgtgaagccccacttggcgtctgatccagtgaaatccctgactggccacttactcagtttctggaagttcccagtctgattnactgttaagccttggatgtcctttgaaaggctggcttcttccaggcttgtttgagtttnatgttggagctgccagctccgcacaatgggtggtttanctgtcctttcccaagcccccaccccctaagtttttctggttggaaaaaaaattaaaggcaaaccaaccaacaggaaaaaanaanaaanggnggnnntngcagnnanaaanngnnnnnannnnanannnnnnnnnngangaannnnnnnnngnannng

SEQ ID NO: 90 - DNA, 964 bp, Rattus norvegicus
cgttaagcgntgaaatnanccctcacgtaaagggaacaaaatctggagctcctccgcggtggcggccgctctagaactagtggatcccccgggctgcaggacgcgctcagccacgtttggacacgggactgacgcaacacacgtgtaactgtcagccgggccctgagtaatcacttaaagatgttcctgcggggttgttgctgttgatgtncntgtttttgttttttgttttttgttttttttttggtcttattatttttttgtattatataaaaaagttctatttctatgagaaaagaggcgtatgtatattttgagaaccttttccgtttcgagcattaaagtgaagacattttaataaacttttttggagaatgtttaaaaaaaaaaaaaaaaaaaaaaactcgagggggggcccggtacccaattcgccctatagtgagtcgtattacaattcacnggccgtcgttttacaacgtcgtgacngggaaaaccctggcgttacccaacttaatcgccttgcagcacatccccctttcgccagctggcgtaatagcgaagaggcccgcaccgnatngcccttcccaacagttgcgcanctgnaatggcgaatggncaaattgtaagcgttaaatattttgttaaaaattcggcgntaaattttngtnaaatcagctccagtttttaacccaanaggnccgaaattcggcaaaatcccttataaatccaaaagaaatagaccgagatagggttgantgntgntccagtttggaacaagagtccacctaattaaagaacgtggactccaacgtccaaagggcgaaaaaaccggnctnaatccaggggcgaagggcccactangggaaaccatcancctaaancaaggtttttnggggggncnaaggngccgntaaaggcncctaaaatccgggaancccnaaanngggganncccccnaatttta

SEQ ID NO: 91 - DNA, 945 bp, Rattus norvegicus
aagntcgaaatnaaccctcactaaagggaacaaaagctggagctccaccgcggtggcggccgctctagaacnagtggatcccccgggctgcaggaattcggcacgaggtttacgcggccgccttgcgcggtttgcgaacccggggaaacctatcctgaaacccaacaagcctcttatcttagctaatcgcgttgggaaccgacgccgagagaagggcgttcttcccctccagaggcaacttgtatcacggagatgtcaatgatgatggcttgctggaagcagaatgaattccgcgacgaggcgtgcaggaaagagatccaggacttcttcgattgttcttccaaggctcaggaagctgggaagatgagatcaatccaggagactctgggacatctggaagtttaccnccccacaaaatgnactaagttgttacagagatttcccaataaatctcatctgagctgaaaatggagaaacattttcaacgaactctcatttctgaaagctacacagaggcgtattagggatgtttgcatgncattgccatgcgtttttgaagggtaaaatgaggcaaaacactcaattttgctcttctgaatgaatcgtgttctggatacgtgtcttgaaataaaaccctctaaaaaaaaaangaaaaaaaacncgagggggggcccggtnacccaattcggccctatagtgagtcgtattacaattcacnggccgtcgttttanaacggtcgtgactggggaaaaccctggcgttacccaacttaatcgccttgcangcanatccccctttcggccagcgggggtaatagcggaanaggcccgcaccggattggcccttccccaaaagttgncgcagctgaaatgggcgaatgggaaatttggaaggggtnaanaattnggtnaaaaattccggggttaaaatttttnggnnaaant

SEQ ID NO: 92 - DNA, 968 bp, Rattus norvegicus
aagcgcgcaaattaaccctcactaaagggaacaaaagctggagctccaccgcggtggcggccgctctagaacnagtggatcccccgggctgcaggattcggcacgagctggatcccaagttcctggtgaacttggacccttctcactgcagcaacaacggtactgtccacctgatctgcaagctggatgacaaggacctccctagtgtgccaccactggagctcagtgtacctgctgactaccctgcccagagcccgatgtgggtcgaccgtcagtggcaatatgatgccaaccccttcctgcagtcagtgcaccggtgcatgacctccaggctgctgcagctccctgacaagcactcagtcacagccctggctcaacacctgggcccagagcatccaccaggcctgcctctcagctgcctagcaaacttggaacttcagggacggccagcagcccttctggctgagggtctcataccacctaccaaacgtcactaggtgttggcttcttagagggccggggctaggttacctttcctgcttttaccttctgccttggagacctgcccgctctccccatcttgtgcagtattgaccaggcagctgtggagctggctgcatgaggctgggggtgttcccacaaggttttccattgtcgttttcccccagagtcagtccccacacttctacagcctttctgggcttccatgtccactcagcagcatgagaactcagggtcccatcaaagcatctctgtgttaaaaccccattgtgctcataatctggagaatgtgggaggacacagggaaanccttcaccatacatacgggntctccagtcaaaangggggttcaggctggtgcggcctaaagggaatgcggaaaanggtgcangnattcagnctggaaattaagggggaaaggattttaaggcntgggaagaaaggggcaagtaagggaattcaggagg

SEQ ID NO: 93 - DNA, 958 bp, Rattus norvegicus
aagcgcagaaattaaccctcacgtaaagggaacaaaagctggagctccaccgcggtggcggccgctctagaactagtggatcccccgggctgcaggaattcggcacgaggcccttcaaatttttactaagactgtgcgttccaaccatgaaatgtagggagtcaagagctatctcactgaggacagggtttgtttggatgctgggttcctcacaagatgggtgatatgtttaacagtggagttctgtaaagtcaccagatgtaactgtaaaccacactgtgtcacaaaaggctcacagcacagcatgtgtgggcactcagggtcagtcggggtgagaaagggccagctcctgtgtggtgtggctgttagagcaacctgttgacctgggggcagaagtgaccagggcagaatgaaagcgtacagactgggaggataagggctagtgctgtcttgagggaccaggacccaagctctccctcagctgtagactagtttggtgaagctggtgtcagcgattacatccatgtcatgattctcgatccagagacaatggccccgatgggatggagccggaagcgtncatcgagagtaactggaatgagattgtggatagcttcgatgacatgaatctctcagaatccctcctccgtggtatttatgcctatggttttgagaagccctctgccatccagcagcggagctattcttccttgtatcaagggttatgatgtgattggctcaagcccagtctggggactgggaaaacagctacatttgcccatatccattctgcagcagattgaaattagatctaaaaggccantcaggctttggttctggganccacncgtggaattgggccagcagattcaaaangggggtaatggcactggggagactaaatgggngncccctggccatgncnggaattgggggggaccaaacggtgcgtgnctnt

SEQ ID NO: 94 - DNA, 989 bp, Rattus norvegicus
aagcgcgaaattaaccctcactaaagggaacaaaagctggagctccaccgcggtggcggccgctctagaactagtggatcccccgggctgcaggtgatgaggggtgagcaatgttactaaaggaaattggtaaaatggcagctacattggctgggtgtcctctgatagtgtcctggaaggggtgttttggattcgcatatactcttcacccactattttgaaaatttgtaataacacccaaaaacgtaaaagttcacgcgattctcttctgctagaaaagattgcagattggctatgcacatagagtgtgtgactagaagtggaaatgcttggaaaggaaaaagagccagggggtgaacaaggcttggtgaatgagactggtaatgtaccccatggaagggaaagggaaagaaatagactggaagggaaagtgcagccctgccctgctccctgtgctttcactaccaggtgcccaaatgcctcagggagacaaggggctgggaagagcaggaggcaacacaggcacaaaaggatgatctgtgagtgagtgcactcccacaagattttcttaggcctgcagaaacgcatgcatccttcccagtgtctctatagcatgtgcctgctactgatgctattcctgacagtgagctcacctggtgactggaggaaatgccagattttgaagcatttgacgaatctgttgccttgtatcattacatttncccatantaatgnaacactaaataactaaggccacagaatgaggtgatnccacaagattagatgggaccgggttgatggtccanctgggncaaccaatggccaatggggttttccanggtgggaggccttnaagnntacagganccaggccccngtaaattaaccaagtggggaataacccggntaccggaagaccgagcctgcnttgggngaanggggganccanggcnggnccnttanggggggaataaaancccgaannccngg

SEQ ID NO: 95 - DNA, 998 bp, Rattus norvegicus
caagcgcgaaattaaccctcactaaagggaacaaaagctggagctccaccgcggtggcggccgctctagaactagtggatcccccgggctgcaggaattcggcacgagatatgctgctgccaaggaagctgcaagtcgaagcaaggactctgtaacccacgagatccaaatgctgcttgcatttcacagtagttaaccatgttaaagagagaatgctttaaaaatagactgttttaaagcccgccgtgcgcactcatcttgatgttactaaagactgtgttccaaacgtctgcgtgcggtaaacccggcgtgctatcctagcctatacgtcatcacaggacttttaagttcattccagatcatcgtatctttaatagaataatagtatttaatttcagtacagaaaattctctgggctgtacactttcagaaaaattctcctcagtgtacacttcagaaaaattctcctcagtgtcttaaccttatttagtattatttgactctaaacttccgtttacctcttgccattccaacacgttacagagaaagtcatctctgccactgtttatcctgggggtcatctgggttcctcctcaagacgcagctcctgcatcaagttgtgtttggntcataaagtcctttgcctataatcataagggnacaccagagtgaacagggaatgttgcaagggtgtaatctcatgcaaagagtagcancggtggcattcccttcctccctgcgggaaaatanggcangaaccantttccntctggtagacctctggccccacggacaggctagggattggcccaagcggcaaaaggccagggnatatggaggctggtaactcccnggctggtaancccnaagggggggnaccncccccggtggganggtncanctggnggcccagnggggtaaagntgggnnccnggttnaaaccaacccaccaggaaaggcccctggatgaaagcccaaaagggnaacccaaggngggg

SEQ ID NO: 96 - DNA, 986 bp, Rattus norvegicus
aagcgcggaaattaaccctcacgtaaagggaacaaaagctggagctccaccgcggtggcggccgctctagaactagtggatcccccgggctgcaggcagcctcagtcgcagccgggcctcgctcctcaacttggcaaaaatgcctacagagactgagagatgcatcgagtccctgattgctgttttccagaagtacagtgggaaggatggaaatagctgtcatctctccaaaactgagttcctttccttcatgaacacggagctggccgccttcacgaagaaccagaaggaccccggtgtcctcgaccgcatgatgaagaagctggacctcaacagtgatgggcagctagatttccaagagtttctcaaccttattggtggcttagctatagcatggccatgagtccttcctccagacttcccagaagcgtatctaaccctctccattcccttccagccaccaagtcatcgcctcctccactccttcccccatccacacctggcactggagcccaccacacctaccacacatgcagcccangcctgacagggaaaataaaacaatgtcatttttttaaatgtaaaaaaaaaaaaaaaaaactcgagggggggcccggtacccaattcgccctatagtgagtcgtattacaattcactggccgtcgttttacaangtcgtgantgggaaaaccctggggttacccaacttaatcggccttgcaggaaatccccctttcggcagatggggtaatagcgaagaggcccggaacggttggcctttccaaaagttgggagctggaatgggggaatggnaaattggaaggnggnaaanaattttgttaaaattcggnggtaaaantttggnaaaatcaggcccattttttaaaccaatnagggngggaaaatnggggaaaaanccccttanaaaanncnaaanggantaggcccgnggaanangggttnaagnggttggttcccaagttttg

SEQ ID NO: 97 - DNA, 1006 bp, Rattus norvegicus
aagcgcngaaattaaccctcacgtaaagggaacaaaagctggagctccaccgcggtggcggccgctctagaactagtggatcccccgggctgcagggcatacattaatattttattccaaagagaaatctttggttagaaagacatacaaaaatggaagctaacactgtgtctgtctgtccccccacacttccagcgtatgcagtgttaagagatatcctggcacttgtatctttgggattccattttggttctcgaacattgggaaaaacaaatggcttggttctgtgtgattaggccaaggttggggaagctagacacctgccattccacagataacttgctgaacgtctacactctgttttccttgcagtaatactgtttcctgccatctccccggcttctggccaccctggcaatagcacccttggcctctagagccattggtccagagtatgcatggcacacctgatggcacagaggtgcccagatatgtcctcccaccttccatatctaccacggaggatgccatcacgccataaaatctggaacccagtgatgaatacatttacatgttaaaaaaacagccacttggtaaaaatcagatcttacttaggataaaggaattctgggctttcatagaagcttcggtagttcagggaagaaaacgccngggaggaagatcattcagncaagctgtttgtagggttaggaaaagggaagtaaaaaacactatctnaagtctgtctgggtcactttcattgaaacacgtttctggcanatttccctcaaagggactctaactggaggcctctgggcagactctggcatcggaccctcaaggtgggaggacctgaccnaagaatctgtggtantttaggggaggttttggtncaaaaaacaattttttaaactggcangnaatttaaaaaaaanatcnatnanttnncgngggnctaaaaatgggcnaattccaaaanggatgggaattnggccnaatggttngg

SEQ ID NO: 98 - DNA, 978 bp, Rattus norvegicus
cgcaagcgcngaaattaaccctcactaaagggaacaaaagctggagctccaccgcggtggcggccgctctagaactagtggatcccccgggctgcaggaattcggcacgagattactttgggctcataatagccaacaaaataaatgtagaggccccatttaaaaaagaaagggaggggtggagtgggagagagggtaatggaaacaaaacaaagaatcaagtcttatgactaagggacacaagaaaaagattgaatgagcagatggaaatattaccacctaggtgtaaacttatgtgtctatggatgtaggttcttcacacacttaaagtacgatgcatctctaatgatactaaatatgtatttataggactttagtgtggagggaggacacagcaggtgattcatcttgaataataaaacaaaaatagccctaaatatttacctggaaagtcatgcaatgaaaaggaattagttttactgaatgttaaagttttttacttatctgtgaaacagtaggaatattaaacaccaatacatgattttnttctcaaagacatttttttaagtcatgcctggtggtgcatgcctgtaatcccagcccttaggaggctggggcaggtttgctgtgaatttaatgtcagcttgtgctatagaacaagttccaggctaaatgtagacnacagagttagaaaacctatctnaaaaaaaaaaantcacacaaacatacacccaaaaaaaaaattgtttagnatagtacacgttcacttcacgtgtgtggttagggcccgaacaacctccagcngntggttncccagggagttggtancttttaagggtaaggaaccccaccagccnggaaccnctaaggaagggcngnagcngggtcaacnagccaagncccntanggggctgnccggaccccaaggnncngggggatgnaaaaaggtaanaaggnggnggaccaannccncanccc

SEQ ID NO: 99 - DNA, 988 bp, Rattus norvegicus
aagcgcgaaatnaaccctcactaaagggaacaaaagctggagcnccaccgcggtggcggccgctctagaacnagtggatcccccgggctgcaggccctgtttgtggtgacnggtcatgaagtaagtcaccaggatgaccctgtctttccaattgcccaccacctgtggctcttttnnnccccccnnnncccccatatttcccttccctgacacctcctgtcccaacctctctaacccccctggggcattttctggcttgcctggatagttttagagaccngcttgttggctataatgtcttttcattcattcattcttctttttctttttttaaaaaaacaaaatgaaacaacaaaaccaaaaagtatccagaaaaaaaaaaaaaaaaacnncgagggggggcccggtacccaattcgccctatagtgagtcgtattacaattcacnggccgtcgttttacaacgtcgtgactgggaaaaccctggcgttanccaacttaatcgccttgcagcacatccccctttcgcnagctggcgtaatagcggaagaggcccgcaccgatcgcccttcccaanagttgcgcagnctgnaatggcgaatggcaaattgtaagcgttaatattttgttaaaattcgngttaaattttgttaaatcagctcattttttaaccaataggngaaatgggnaaaatcccttatgaaatcaaaagatagaccgnaganagggtngagtgttggntcccantttgggaaaaagaagtccacnattaaagaacgtgggacnccaaagtcaaaggggggaaaaaaacggtntaatcaggggngatgggccactaaggggaaccaatcaacccnaaatcaagttttttnggggggcgaagggngccngnaaaangcactaaaancggggaaacccctaaaangggnngccccccgnattttaagaagantgangggggaaaanccgggggaaangngggngaagaa

SEQ ID NO: 100 - DNA, 971 bp, Rattus norvegicus
aagcgcggaaattaaccctcacgtaaagggaacaaaagctggagctccaccgcggtggcggccgctctagaactagtggatccaccgggctgcaggttccccccctgtttgggtgaaagtggttctagaacctgcactgaatagtagtaaagcaataaggcccgattcatcccacagcactgatcatctttcaatgccccaccccaagcgaacggtaagaaggcctctcttaagaaggggagacagatggccctaactactcagtgacagaggcagttactgtgagagacttgtaggaatccttttctttcctagcgaagtcaaagctctctctgaatgtactgtgtgacaatgcatcatggcatgaaccttcggtcagggacgtcattggggaagtgacttcaaaagtattcaaaatttgacatgctgtttgtttagtcactacagtgccctcaaagggcagacattgcagcctttttatattgcctgccaaaatttgaagtattagaacaaagtgtgccatgagagaaaaacttaacaaggagttttgaaaagtaatgcaaagaacaaaactacaacactatttttaaaaagttgagtatctgagttaaaattttcaaatctttattttacaccacttaaaattatacgagaacaaggtacatgcattatgtgtcacattactgggcaaactgttcaaatattttttttaaaccnccctgtatagaaaatatcattaagggatgtaaaagccatgcttgcctatttgcngtatacatgtaatgaaattggtagataaaaggggtagtgcattggaaaccaaatggaacaaaaaagtagntacttttactataccaagggtgccnggtgcaggaaaaaaatataananaanttngtgggnaatggnagcantttaaaaccnttccaagggggtataaaaaaaaaaagggggggggcccgggaccccaaan

SEQ ID NO: 101 - DNA, 1006 bp, Rattus norvegicus
agcgcgaaatnaaccctcacgtaaagggaacaaaagctggagctccaccgccgtggcggncgctctagaacnagtggatcccccgggctgcaggcttaaatagaattcaattctaaaatcctaaaacccatctccgggttaccaaagaatgatcttacaggtcctgtcagtgttctctgtgtgatggttctgagaattaaaacaactatagtcctctgtgattcatggcttctctccatgctatcaagactatctgaagaggttatagaaatccttggccggtctgcacctcacctgccctccaggacacatgtgatgtgtgcatccagatgaccaaaaacagacccttagggcttcgataagggtttacaatagtttgaagtgctttcgctcacagctagggaacctttacaaaccaagctagcaatttaacnccttgctcttcatggggacacatcttgggcttgaagtcagcagtagagtcaggagaaagttcccaaagatgtttgagcaattcaatttcatcccngctcccattgatttagacttcaggtttcgaggggagcctactaaagtatacattaaggataatttgcttagggcatatcnaatngtggttgtccgagtagatgaacgcagagtctctcagaagtccacacccatcacagcaacaagtaagaagctcataacagaggaacatctactattngctttgtnttaaaaaaccccccagatggcnaaancatcctttctgtttccanncngntncgcctncaagcnctggngtggtgnggtgnntgtggtgngngngngatgagngagtgtngtggtggtgnggngnngngngtntgnnggggggggannggnaagcaggtccagggnagaatnccacaaagggggcaagaggggganggggtccagnanaanccccggaaangggggagannacncctggggaggggnnaaccaacaanttcccnaanggnnnttnnacccagnaaanana

SEQ ID NO: 102 - DNA, 968 bp, Rattus norvegicus
agcgcggaaattaaccctcacgtaaagggaacaaaagctggagctccaccgcggtggcggccgctctagaacnagtggatcccccgggctgcaggaattcggcacgaggtcccctggagctgagtccctcagagtggggtcttcagtagggtctcctacagtcagagaggggcctgaggatggaccagacagcacaataagtgaggctgccactttaccttggggaactgacccccatcccagcgccccacttccggatcccccgggctggcgagatattgggccagagctcctagagtcagaagcacctattaagtcggaggaaccactcaaagaggatgccaacctgctccctgagaagacagttagggnccttcgtgncccattgacctacagtggcatcgagcggnaagcctncaggaggagcgcattcaagcatcgtgagggccggaccaggcgagctcaggaatttcttgccagcccgactcagccaccctgagcccccagagcgcaatggggctgaggcagttgtgagaccccctggccggtcctgcgggggctgtggaagctgtggaggccgtgagcactgagagctgtagcctcggtggtggctgccctcatcttcttcccctgcctgctgtatggagcatacgccttcctgcctttcgatgntccaaggctgcccaccatgagctcccgcttggtctataccctccgctgtggggtctttgcnaacttccccatcgtgctggggctcctcgtgtangggctgaagctattgtgcttttnngccctttggccctttggaagagccgagaagggaagtagaagattccacgggcagtangtgggcccaagttctgtgcaggttnttnaannctctantttctttaaacctgggctgtggcttttcaacctaanntggncccaaggaanaanggtnaaaggntgganccccccttggnnaa

SEQ ID NO: 103 - DNA, 1033 bp, Rattus norvegicus
aagcgcgaaattaaccctcactaaagggaacaaaagctggagctccaccgcggtggcggccgctctagaacnagtggatcccccgggctgcagggctgggcttgccttctgtggtaatacatgacagaggcattagacttttttagctgccctaataagtatatgatgaataaataaatgccaatacccataacaatttttctagtggggacaaaggtcatttgattaatctaggttttcataccgacccgattattaaatgcacttaaaaaaaaacngcatttaaagcattggccttagcagaattaactgacttagttccttacnggtgagtgaattcagttccctaccaactaatgcgcagaatctaagaatcccatcctgcacacattggttgggagccctgagtggagtatcagcagcaccaactggataagaggcacaagagaagtggggaacatctggagtcctgttggacgtggacagtgtttcttggatatgtatgccacagtgccttggtggccagcttggagtcctacctactcagcctttgtccccacncctccngtgttagcttncatcttgcatattcnattttttncnactttgttaaccnactttatgncnttanctcaataaaaangcntaccaagggnaaaaaaaaanaaanaaaaaaaaaggggcccggnacccaatnncgccctanagtggaggcngnaatnancaantccacggggncggtcgttttaaccaacggtcgtnaaggngggaaaancnctggncggntanccaaanntaaanncggccttggcaagcancaaaacccncctatnacggacaagaangggcgngtaataagacgnaaaagggncccgacaaccgnattaagccccgtncnccaaaaaantttggggagccctggaaannnggcgaaangggacaaatnnntaananggntaaanaaanttggganaaaaagtcgcggtgnaaaaanatnnnggaaanaactggcaccgntttganaccaaaaagg

SEQ ID NO: 104 - DNA, 1011 bp, Rattus norvegicus
agcgcggaaattaaccctcactaaagggaacaaaagctggagctccaccgcggtggcggccgctctagaactagtggatcccccgggctgcaggtcagaagcaaagccactgtggcagagaggaagcccctctgcccctccgcccccctgcccctccatccctccgctggtgtttctggggattattcactctccttttcccttcacaagggccttctctgcaggagcgatagagaatgcatgtctgccccattgggccttttggtctgggatncctccaaccacatgacctataccccaagcccgcctctccatgcgcttcgccccctggatgcactaagagttgctctcgtttgttcctggtctggatggcaaaacaaggagatggttatttaaagagaattcctatttatttggacacaaaaagtccagttaatatattaatgtgaaataaaccctgtttggcaccttgaaaaaaaaaaaaaaaaaacncgagggggggcccggtacccaattcgccctatagtgagtcgtattacaattcactggccgtcgttttacaacgtcgtgactgggaaaaccctggcgttacccaacttaatcgccttgaagcanatccccctttcgccagctggcgtaaatagcgaanaggcccggnaccgattcgnaattcccaanagttgcgcanctggaatggcgaatgggcaaattggtaagcggtnaaanaatttgttaaaaaattcggggtttaaattttttggttaaatccagnnccatttttttaaaccaatangggcggaaaatcgggnaaaaatccccttaataaaatcaaaaaggaattangnnccggagnatnagggggttgagtggtngttcccantttgggaacaangagncccacctaattaaangnanggngggnnnnccaaaagggcaaaanggggggaaaaaccgggctnatnaaggggggnatgggcccaataanggnggaanccaatnaancccnnaatg

SEQ ID NO: 105 - DNA, 1013 bp, Rattus norvegicus
ctcaagcgntgaaatnaaccctcactaaagggaacaaaagctggagctccaccgcggtggcggccgctctagaacnagtggatcccccgggctgcaggcggcagcgaggaaaagcctggctgggcccaggtttcatgtggngggggagggggcaagccttgcgatcagctctgtaataagcgtagcaccggggaattcaagaggccctgagaagcctggagtcccaggacgtcttacttcttttcctacacatgtctctgagccatgtttttgcttaaattctctctcaagaaagacacacactaaaagtgaaatctaacagccgcaaaaggttacttttttttgtttagatggttttgtctgatttcattttgncctggaaagttctgaaataagaatcaaagtatttaaagtctcgcgacacgacagtgttnacaggactggctgtaaccgtgttatgtaatcagagcgctccaacaagggaaatctggtaggattccattggaacgggtattggagagctgaaccaggcaggtggtggtaaggggttgccctaagagtctgttacacaatgcggtgtcatgggaatttctcagagccatgggaactctcaggaaggcaaagagatattctaacctggagagcacagggtccccaggtccgggtctaaggctggacttgtgatgcacaggtggntatttccagaaccttagggaattaacagtttccacctggcaancagcntgcctgnacnaaatctatnggattnncnaagcngaattaatcccagtccaagggaagcaagcccaatcnggnnnncttnccgggggcagnaanctaatggggcggangaaaagaaggnngaaaancacaaaggccccaaaaaggaatgggncttggggtaaaggnnggaacccaggccaangaatcgnncantgaaaaatggggaaaaaangggnggngntggtgaacgaggatatggaaatncnnacatgaaagtttaaagancaatc

SEQ ID NO: 106 - DNA, 989 bp, Rattus norvegicus
aagcgcggaaattaaccctcacgtaaagggaacaaaagctggagctccaccgcggtggcggccgctctagaactagtggatcccccgggctgcaggccactgcaaggcgtcagacagaactagtatcttcacacttgacaaggagncacactgtcactaacagtgttgtgtggccgccctgtgggtcagtgctccggcacacagggctccctggaaagcgatggagatgaaggagctggagaacatctctgtcccggtgagcggctgggctgtcacctctgctgagcacggtgctttgctcacacggcttaccgagatgttacattcacacccagtgaacctgggctttatgagtatttttatgaatgctgattgcgattatgnatttttgttttgacctttatgtgtgtgtccctgtgagttgtgggcgcctgcaaggatcagaaaatggtatacgattcactggatagagttccgggtggtcgagggcctcctggaagtgggaaccaaactcaggcagaagaaccatcctcaagagcgctcagcctccgagccaactccccagcccccttggttgttatgtttccaacacacagctcaatggattagaatccaggggaaaataggaaacaatctctggactctgggggactgaaaagtctattaaggatcttgtgattcttttggaccatcagaaatcgtctttcctaaaatagacatggatgttctggttaaaataaaaacaacatatcagaaaaaaaaaaaaannnnnnngnganaaaaaaaancgcnngggggggccccggnacccaaattcgccctaaagnggaancgnattaacaattcantgggccgccggtttnanaacgncgtgacctggggaaaaanccctggggggtacccccaactttaaancgccntggaagnaaaattccccccttttcggccaagnntgggggnaaaaggcgaaaaaggccccgcaccggntc

SEQ ID NO: 107 - DNA, 1020 bp, Rattus norvegicus
ctcaaggcgaaataaaccctcacnaaagggaacaaaagctggagctccaccgcggtggcggacgctctagaacnagtggatcccccgggctgcaggggctgcggagaaggcagcgccggacccagcggtgcggtacggcaaccacggccgcacgggacctcgggccggaganccccggcggcggccgcccctcgcgccggggccgttagtcagcagaacgcgaacgcgcgtgaaggggccacacgaancgcgcccgggcaccgcagctctcattcgcgttctcgcctggccgggccgcgcgcggagtcggcgggcggcgccccgcggaggaacagcgcgttggacggccgcggcccccaggctgcagaagatgaataatctttcatttagtggagctatgttgcctcttctgctgtgccaccttgacccaaggaaaatcgttcaaaactaagcattctngncacctgatccaacttacacactgatgtgtgatgagagtggaagccgctgnacctttanacctgtcagaangancggantggnagtantngtncaganagaaagatggtattggagtgttntcatgnacgagaaancagtaanagnnaacagaattggcttgtangtttgttgcggtgntnaccaaatgntnataatatgaccttnacngntcttcagagaggaatgacngntgaatctngggttcagaatgannggagttttttaacatanggnactggggnggtcccgggaatgaaatntgtgaagtaaatatnncngcnatganngaatnnactnnngggnatgagggagggctaagttcgtgngtaaagnncanacnaagaganagtnaanntggnnagtgncaagancaagtagaaancgngacngngnggganagcgtnnanngtggacanaagnantacaggggaannnnggacnatngnaaaanggcgaaatngaanatatgaggacaatgaagnagngangggggancggnaacangggngntgngtnnnc

SEQ ID NO: 108 - DNA, 917 bp, Rattus norvegicus
gcaagctcgaaattaaccctcactaaagggaaaaaagctggagctccaccgcggtggcggccgctctagaactagtggatcccccgggctgcaggtcaaaactcagttctagaaggaccttgacaaacagacatacttacctcctggttagtggaactctgggacaatgtcctttgtgatggtgttttcaggagtccttaggttatgttgagaaaactcagtgtaggccaaaccaaaactgatggttctgtttttgtaattgtctctttcatgtcctattatactctgtattaaaaaacagggttaaaaggccatagtccatttcctaaaatgtaatgcgcaccttgtatctgtgaggtggtctgttggtgtttttatccgaataggttgtcaggtgaatatgaccccttcccgttacccagagggtatattctccaggtggtcagagtgcaggttatttcttacacagagaacctaccttccctccatatgattcttgacctctgccctcatctcccatttaataatttaaacaaatctgaggccaggtgttgtggcacagtggcatacacctgcaaatctgcacatgggaagttaaagcaggaggatcaggaatcggggatagccttggctacagagagagttcaaatccagtctggtatacatggagtcccagcctcaaaaccaaacacaggggcgtctggaaggaagaactctccggctggtgctgttcagggggcactggcagagtgtctgtgcccagtcaaaggtggttaacccagtgaacagataanctggaaatggacagtgttggatagggaaataagggtctggtgccaccatgggagtcaagtcanttgaccaattcctggacccaaagcgaaggcttgantaanaccaccggtggaggcctacgg

SEQ ID NO: 109 - DNA, 895 bp, Rattus norvegicus
gcaagcgcggaaattaaccctcactaaagggaacaaaagctggagctccaccgcggtggcggccgctctagaactagtggatcccccgggctgcaggaaggtgggccactctctgtccacattgcctttattttcttggattgaacgcataacgtggcccctcacattctccaggacctgaccagctgtgggccactgactgcttgcaaacccggactgtgctaagttactagcgtgtagcccttggggacccacctggcccatctggacacatctcaaggctccagcgaggatggatgtaaaaatatttccttgcttgcatccagattgctcatggatacggggctgaaggcagaagcagctgtctgggtacgacatggagggggagctgggtcctgctggccgggatagctcagctgtggactttggtctctggagtggatgtcctggtcatgttagcaaacattcactgccctttctcagtgccctcgctctctcgcctccacgttactcccgcgctactcttgccgtttctcgcccgcgtttctgagcacaccaggtcctgcctggagtcttggtgtcgcggatgactgactgaaggggcctttgagagctgatgggttctgccatggactcctcccggtgattagcaatgactggggccttacccacccacctaccctcgtaatgaagttctgtggagtggctggacaggtttgagggaaggtggaggtggtttaaactggtttggggagtgctagggctggggacccagaagcaagcccagggtgtccccaaccctttcccgcanggtcttgctaaatgttctgatctctgtaaaaccccntccctctttcagaaggancctggggtggggcccctctgaaattcccna

SEQ ID NO: 110 - DNA, 901 bp, Rattus norvegicus
aagcgcgaaattaaccctcactaaagggaacaaaagctggagctccaccgcggtggcggccgctctagaactagtggatcccccgggctgcaggtttatatttaaaaacaattgtcccttaattcatgggtacaccttgcagttgtaaattgttttttctctagtacacatcctaatgatggtagcatctgtatttctcacatataaagaaaatgtactaaaattcgtctaactccattactagagcataatagaggtaacttcaggttcctccttttctctgagctctttatcctgggcgtggctggctcctgcctcgtctgctctttgcatctctgtatatagagttacacaggcctggaatgtggtcctgctcactaacaggcaggtgttcactgtgtacctgcaccgcctgtctgcagagtattacgtaatcggcgtctgaccaggagctggatttgagtcactcagtctggttcagggacactgggaaacatggcaccagttcttcagcccatccagatataccccgaaacaagtattctttttgttctacctacagccagcaagcttttctgtaaacggccaggtagtgttttaaggtctgncaagtcatgtagctctcttaatactcctttctgctgttacagctataaataatatgcaaatgtacgagtggagctgtgatgcaataaacctttactaaaagacagacggacggaggcctgcatttgtcccttacctcacagctgctggccccaggccctaattcaatttaagacctctaacctttaanctgcatctttccccctttttaattgcataattcacttnaaagcagaaatgtgtgtggtacacncggngggggaatncccggggncnggaancacctaaaaggctg

SEQ ID NO: 111 - DNA, 924 bp, Rattus norvegicus
tnncgcaagcgcggaaattaaccctcactaaagggaacaaaagctggagctccaccgcggtggcggccgctctagaactagtggatcccccgggctgcaggtttggcattaggccaaaggccatagcaacagaaggttaggatatagacactcaaactatgatctttaaagtgggttgtatcacttaaaggacttggttatatccataaactgggaatggtggctgtgtgatacttcatcaagtaacagatgtgggtgtatcctacttggttactctcctgggatgttaacttccttttctaggtgacagatttcttcacataataggaataataatgtatgtgagaacttactctgtgccaggcagtgtgtaggcacatacacagaccactgcatccccttagcagccctgtgagaacacatcggtctgcataaagcattgnaaacaggaatatgcatctcgtgatgaggaatgatcgattatacctatcaattaagataaatcaataaatcatataccatagtttatatgcctttgaaaagctacatataatcactaagggttttttcttaataagttaaaatgttagttacagcctgccagtgtctaacatttggaggctgcagctttcagaacacttggagtaactactgaatgggccattgagctttcttttgctgcctataggaaaacagtaccttantaagttttacccttcctacagtcacttgttaaatggcagttactgacaaaactttaacaagtacttgctgctgcctgtcnangctctccctgccnaaagggcctgtnccatgcacnaaatccgncattaaacctagggtaggcctcaggaggtggggttncggtnggacctnaaactngcccattccaaggaaatgggggaacnacnattaccggtgntc

SEQ ID NO: 112 - DNA, 893 bp, Rattus norvegicus
aagcgcgaaattaaccctcactaaagggaacaaaagctggagctccaccgcggtggcggccgctctagaactagtggatcccccgggctgcaggttccctccctctccttactatggaattttcttgttgtttaatcacaaaaacaaacaggttctgtttctctcctcagggtcggttttcagtttttgggttgggtttgggttttgggttgggtttggggttggggttgggtttggggtttggtttggttgtaagtttggggtttggtttggttttttgttttccttttatgctctagagaacaaggatagaggtggcagctgagatctttggaaccaaagcagagcatgctgagtccatgatctagggcactggccaccaaaggcagggccctcacatatgncccataagccagagcccccatgctgtggccacatcaccctgtcttctccgtactgcatcctcttctacgtgctttattagctagtccaaagggggcagcagtagtaatgagagctgtgtgagcacatggaactccatgcactgaaacccacataccactcatttagaaaaagacctcgtaggcactgcacaaccagccagctctgctgcctgtgtctgcccggctccctcctccttagattgcttgcctgctaaacccatggctagaactcactgtctggcagaaggatacccaagcctgttctgggattttgtctgtcttcagccagatgcttcctgcccttccttccttcctcctcatattgctgtttctcnctggtgtgcaagttctgngccatttccatagcactgatcttaacacccagataaagcnaagggcttgggccaaagatcanctttcnncnccttacaganggngaaaccccaacct

SEQ ID NO: 113 - DNA, 1012 bp, Rattus norvegicus
ngcgnaagcgnngaaattaaccctcactaaagggaacaaaagctggagctccaccgcggtggcggccgctctagaactagtggatcccccgggctgcaggaattcggcacgaggccagttccagattcagtcttctctgttagaaagaattttctacttggtcaactagacatacctgccagccatatgctgacagcgtttaaattcagggcttactccatatccttataagacctagcatatgtgggcttcgaatattcaattacttgttgttgccagttttactaagcttagtcaaataactcactctttttcaataggattttatttcattcaagggttctttgaccaatgatcttaaagggtggtggtgttggggatggagctgttaggagcactggacatctcttccagaggtcctgagttcaattcccagcaacacatggtggctcctctgtaatgggatctgatgacctcttctggatctgagacagctacagtgtacttatatacataaaanaaatctttaaaaganaaaaagggagtattatatttgcttgctaataagaaaaatcttattttgttgttgtttctaaaaacgtaagatcagtttcttagtttggcctacattttaaaaaccgtagtgattccgagtgtgggtggaatctcccatggaagcgtactgttgagaagaggctgagttttggtgtaggcacatcccagccagcaccgcatggaccanttggtcacgcttcatgtgcctgccccaccntccananatgggnttccnggtngccntggnntccgggtcngggaaantngaaanccccagaccccctggggtttggcgagaaaaaanantggcantaactggagccggnggaancccccaagncccctggggnagaacctncctnggaggnagaganggccaatttcggaanagccnaacnaattacaattggaaggtnntaggnnccngggggngnacnaagaangnngaaaagggggc

SEQ ID NO: 114 - DNA, 993 bp, Rattus norvegicus
cgttaancgnngaaattaaccctcacgtaaagggaacaaaagctggagctccaccgcggtggcggccgctctagaactagtggatcccccgggctgcaggttagtgaattcagggctagccttggctatatgagaccatctctcattcaaacattaaagatttttcatcctttagtatgtgactgtcctgttttctctagactactttaaatatctctactctgatgaaattaagggaattgtaagtttcaagttccaaattacagcattgagtctagaaatgtagccaaactccatattttcatagtccctgtttttatttcctacataaacngataaattttccctgtacttaagggctttggcagtaattctgtctatatttaatctatnggcaccctctaaattgtgttttctttcaacacagtcatccnggccaaatcctttaaaatgtttttgcttcagtaaagaggttgaagagctcctatttaaaagggcagttgaatgtcaccagcaacagatgcaaagcctaggttcaagggttgtaaagaggccagcactgtgtggtgcagatttcaaggcaccctgttgatcacnggggagagactggctgtcacacagtcactgggacagatggacttgggactcataccaaaggcttctaaatcagaaataaatagtataagggcatctgtntccccgaaccttcctaggtccatgaaaggtctccagctcactgttactgttttccanttcancngacgcttagatgagtagagatagaagttggncattgggaagctaagtagagccttagtcngggctcnctagaaggggcaaaaggccagccccaanggaattaanctaggaggctcaacctnancngtccccattngggacnaantccataagggaattgggcacccagggcttttccnttngaaaagcnnccagccttggntgggggggganaagaaggttgaatncccntnc

SEQ ID NO: 115 - DNA, 997 bp, Rattus norvegicus
aagcgcngaaattaaccctcactaaagggaacaaaagctggagctccaccgcggtggcggccgctctagaacnagtggatcccccgggctgcaggctcgagagtcatcatggatcttatctttgtaacacacttgtcaacctacagtgacagagcatgtgtgtcattcaaacagcaggacttccaaaaacatcatcatctacattgtcaagttctccatcatggagatatgaggccctgaaaagtatagatcaggggacctgtggagacttgaggttccagagtgaggagagcactggctgcctggtctcatgcctgtctcatgggaccctaggaaggacgatggaacaacactgttctacttgagggctatgncataagctttctcaaaacactaccacacactggacatcccctcactctggcagtcgtttacactactggcccaaattcctcagctgtatcaagaggctcgacnccaaggcctttatgacagaggggaagactccaggtgaaggactcagaaggagctgctcagtgctggggactcatgtgtagcctcaaatctaattgtgggtgacctgctgctctctggggtcagtcacagagcagctgcttccagacaagcaggaagaaggaacagactctgtgcactttagcacaaaggactggagccaagtgacagagacatgggacagtcccaagtgtttccccagataacccancctcttcctttacctttcagaaacanttttacnctgcaaaggaggtcttggttaatgtctgaaactggtggcanagagggattagnntgtttaatttccaggagaagcataagtttggtnaancaccttttangctggaaatggaaatttaggggaggttaattannaacntcaacaaaactcccaanccntttttananccttttnggttttnanagagnnnncaccataaaccccgggnnggaccngganaannggaaatggaaaccnagg

SEQ ID NO: 116 - DNA, 979 bp, Rattus norvegicus
aagcgcgaaattaaccctcactaaagggaacaaaagctggagctccaccgcggtggcggccgctctagaactagtggatcccccgggctgcaggcaggagtactcctcagcctagcttgggtgcccctgatctgctttgatccttcccaccaagcaaatgaattgtttagcaaatgaattttgcctgggagctgcctcccttccccctaggagtggttggttcattgagtctgctaacattcttcaaaaaattactataaccaagaaagaggaggctacctgtctttgaggatgccaaccatggcccccatcagcacaggtcccatggcatggtgagcaatagcagaggggtttaacaagggaggaaaagacagggtctgccctgcaggatcctttctggctggggaggtgttggtcaagggagaagtggtactcctggagtgacacccatggcacccaggagtatggtgaccagcctgttagggccccagcaggcaccctttgctcaaccctccaaggagttgcctaggttccagaattctgctgtggttggcaggttcaatctggtctccttcacttccatattagaagtgtcttttcctcattgcggaactgtgcctcctcgggggacagatgcctttagatgcaactgcttcaaggaccccaaaggccattcccacaatataagcaatgacttctttttcctttttagtcatgctaagtagttagacaatccttgaacttgggatctctggttctggtgtaagagccaattatgttttaactactcattgggaagagctgagggtttccagtggtgtgtcctaaagagaaggctgaaggcttgccaatggtgttccataaagagaaccccagttggnaaaattgggggatgccaagccggtttttggnaaaggttccaaaaccaagggggantngncaattgggaaanacccagccaccaggntnaaatgggggn

SEQ ID NO: 117 - DNA, 979 bp, Rattus norvegicus
aagcgcgaaattaaccctcactaaagggaacaaaagctggagctccaccgcggtggcggccgctctagaactagtggatcccccgggctgcagggtccaggccggggacgtgatcaccatcgacaaggccactggcaagatttccaagctgggccgctctttcacacgtgcccgagactatgatgccatgggctcccagaccaagtttgtgcagtgcccagacggagagctgcaaaaacgcaaggaggtggtgcacaccgtgtccctccatgagattgacgtcatcaactcccgcactcagggcttcttggctctcttctcaggagacacaggggagatcaagtcagaagtccgagaacagatcaatgccaaggtggcagagtggagggaggagggcaaagcggagatcatccctggggtgctgttcatcgatgaggtccacatgctggacattgagagtttctctttcctaaaccgggccctggagagtgacatggcgcctgttcttatcatggccaccaaccgaggcatcacccggatccgaggcaccagctaccaaagtccccacggcatccccattgacctgctagaccggctgctcattgtgttcaacatcgccctacagtgagaaggacaccaaacagatcctacgtatccgctgtgaggaggaagatgtggagatgagtgagacgcctacacagtgctgacccggcattgggctcgagggggggccggtacccaattcgncctatagtgagtcgtattacaatttcaatggccgtcgttttacaaagtcgtgactgggaaaancctggcggttacccaaacttaatcgccttgnagcaanatcccccttttcggcaagctggggtaaataagcgaagaangcccggaaccggattnggccnttcccaaaagnttgcggaaccttgaaaatgggcgaaattggcaaaatttgtaaggcggtaaatanttttng

SEQ ID NO: 118 - DNA, 989 bp, Rattus norvegicus
aagcgcgaaattaaccctcactaaagggaacaaaagctggagctccaccgcggtggcggccgctctagaactagtggatcccccgggctgcaggctggccacttgctcataggccagggctctcaaatgagcacctttttaagggtccttcacccactctgtgctctccacagaggcttccacgttgctacataatggacacactccgatatagccaatgggcaggaaatccggggcacttgtgcagggccggggatgggactggggataagggaaagattagaggacaagggtaagatttttatttttgggtgggttgggtaagacaacgtatttcagtaataaaatacagaatggaaaaaaaaaaaaaaaaactcgagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaaatagcgaagaggcccgcaccgatcgcccttcccaacagttgcgcanctgaatggcgaatggcaaattgtaagcgttaatattttgttaaaattcgcgttaaatttttgttaaatcagctcatttttaaccaataggccgaaatcggcaaaatcccttataaatcaaaagaataggaccgagatagggttgagtgttgttccagtttgggaacaagagtccactattaaagaacgtgggactccaacgtccaaaggggcgaaaaaccgtctatcangggggatgggcccctangtgaaaccaatcaccccaatcaagnttttttgggggcccaaggtgnccgnaaaaggacctaaatcgggaacccctaaaaggggagccccccggntttaagaggcttgancgggggaaaacccggggnaangtgggcganaaaagggaagggganaaaaaccgaaaggggccgg

SEQ ID NO: 119 - DNA, 978 bp, Rattus norvegicus
aagcgcngaaattaaccctcactaaagggaacaaaagctggagctccaccgcggtggcggccgctctagaactagtggatcccccgggctgcaggcagcataactccttgaggacagagagttttgttactgcttcttccataggacttgggccagtgctgagcgtgtgaaaagcactctttgactgggcagacaggagggcccacatgggccatgcaccattggtggcattgggaccaagcgtgctgcggccagaacttagtctgagggtatattcctctccgccacaggaacagctctcactttcttacggttattcttagtttgttacacatgactcctctgtggagctctctgacaggctgaggtcctatgaagtagggtggaagagaatagctacagaattgggcctcagcgttcctatcgcttgagcatccagtcagggcaattccggcaggctgcatcatccttgattgttacaaacactaatgaagaaaggcagcattcctgtgattttaaaggaaacacagaattttagcttcaagtatgggcattcctttgtgaaacttctcaggaaaatgttgtttctaagtaagtttatctgagaatatagggctgttacagaatgggatgctgttctgcagaaagtcttttcattccataagaaggaatagtgatattatacaaagaccgggaagggttccctgttaaagtatcttttatnctcctgttgtaatgtagtcttagaggttcactgcctctgtctcctaacctagggtcctggaagcttctgggcctctgtacagtctaatctagggcctagaaaggtttccaggctcggagaattcaatggcngaaataagctcaacccttcccaagtccnttccngaaangaaacnccttggcccaggactcccncctccaagggctggacnnggancnaaanccnggggntccccccnccnnccnccnggaaag

SEQ ID NO: 120 - DNA, 992 bp, Rattus norvegicus
aagcgcgaaattaaccctcactaaagggaacaaaagctggagctccaccgcggtggcggccgctctagaacnagtggatcccccgggctgcaggaatcattgacatagaccacaatgtagcttcacgatgtgagaatnccttctctaccataaacacacatgactagggatttgccttctgaagtctgggactttgggaaaaggaatcccacactgtggtttcatttttgcattcactatacacagtaagtaaactaagaacatgttgtaaccatgtatttactctgccaagtgcctatatgccaataaaacattcatcgctgaagggctgtccagatacctttatttagaatggggctggcttcatctttaagcaaatgtcacactggagctgcaggaacagcccttagaaatgaaaggggcaagtcactacctggctggactctgggaaatttcacgncctncttgttgttggggagaaatccactggggcacgacttgatgtccaatgaaattctgctttgataacacacatggcttatttttcaagggaatggatcaattccacaaaccagcaatggggcaattactcttgatataattgaacggctgttcaaacttaatanattttcagcgggcgagactggagtaaaacgttccntccanctgtaattataaatgagacagtgtttacttactaaaaaaaagaaaaaaaaaaactcgagggggggcccggtacccaattcgccctaaagtgagtcgaaattacaattcangggncggncgttttaaaangtcgngacngggnaaancccnggcgttanccaaccttaatcggccntgngggnanaatcncccctttncgccancngggngnaaatancgaaagaggccngnnaccgaatnggnccataccgananggtggnncancnatggaatggngaaaggggaaaattggaaanggggtaaaaaatttggnnnaaaaaancgg

SEQ ID NO: 121 - DNA, 983 bp, Rattus norvegicus
aagcgcgaaattaaccctcacgtaaagggaacaaaagctggagctccaccgcggtggcggccgctctagaactagtggatcccccgggctgcaggatgaagatgtcatttaattatttgccaaaactagggttttaatgaagtggcttccccagtcccctttttatagttattttctggtgtagacccttgaggtggctcagggtataaaggtgcctctcccaagcctgacaacctacgcttgatccctggggttcacactgtagaaggagaggactaccctggtacattgttctcagacttctgtatgtgtgcgcgctcgcgcttcacacacacacacacacacacacacacgcgcgcgcgnnncaaagngcaaattaaaaggtatataaaagttaaatttctgttttataaaacggtgtttaataagaaaatatttataaaatttaagtacaaaattatctattaaaaattcctattcctgccagtcaatggtggagtttgcctttaatccaagctctctggaagcaaggcaggttctctgaattggaggccaacctggcctgtggagtgagttccaagacagctggggcgacacagagaaactgtctcaaaaacaaaaccaaatcaaaacaaaaacngctattccagttgatactgataaccatgtaaaagagagcagaaatatcatcaatacaatatcgtccctttgggantccctgggggggaagcagattgtatttgtgtcattggtatctgccctgtttctttataaataanactctagaattcttgccttggggtttgcagagttgttgagaaggaaactggtcgccgtgttttttgggaaggtggaagtggtacctaaggattaattaatgaagganaacgggngnanctccnaaggnaaagnggcggtggaagggaaancgcctagngggnnttccccggctttntnnnnnnnggggntnggnannggcccnc

SEQ ID NO: 122 - DNA, 973 bp, Rattus norvegicus
ncaagcncngaaattaaccctcactaaagggaacaaaagctggagctccaccgcggtggcggccgctctagaactagtggatcccccgggctgcaggcgcagtggcggccgactcctttcggggcctaaagcagctggatatgttggatctgtccaataactctctgtccagcactcccccgggtctgtgggcgttcctggggagaccgacccgcgatatgcaggatggtttcgatgtctcccacaacccctgggtctgtgacaaggacctcgtggacctgtgccgctggctggccgccaaccgacataagatgttctcgcagaacgacacactctgtgcggggcccgaggccgtgaggggacagcgactgctggacgtggcagagctggggaccttgtgaggatggcaactggggtgcgagccaagggtaccccgcttgccactgaagcaatttggtcccatgtcagaatgcagattcccagcatctgccattccccattccctcagccaggaatgctattccctgactctccctcaggctcctctccatttgccccaactcttccacctctcactgttcctgtgctggcccccaggctaccatgtgtttatctagctttgcctcatatgtttcagggtcaccaaagcagttaataaaacagctcccggctggctgagccgctcaaaaaaaaaaaaaaanaaaaaaaaannannannnnnnnnnnnnnnnnnnnnnnnnnnnnnnnanaaaaaaaactcnngggggggcccggnacccaattcgccctaaaggggggngtattaaaattnannggnngnngttttaaaaggnngggnantggggaaaaacccngggggttacccaantttaannggctttgnngnaaantccccctttttggcaannnnggggnaanaanggaaaanggcccgnnannggntnggccntttccaaaaannttggggggnttg

SEQ ID NO: 123 - DNA, 976 bp, Rattus norvegicus
aagcgcgaaattaaccctcactaaagggaacaaaagctggagctccaccgcggtggcggccgctctagaactagtggatcccccgggcngcaggtttctgggtagaaacatgcattttgcttactgagtagagaacaaagtttacattggacttgtggggcaggacgacaggggaagcttgggaagccagcaacctgcaggaagagtaagccggtctaccaacaggttttgacctttggtttcaaaaataaactcaaggtgctagattcagtatcaccaaggttttaccatgtgaccttgggcaagtcagttcctctccttgggctcagttcaccctgacctggggaaatcagacaccaggattaggtcatctttcatgaccccttccagccctggacattccatagggaccattctaatcagttggcatgtgccaggcacccagcaggaagctgcctgtggcatttccatttcactgaattctccttgtaaccctttggctttctaaggtgggcaccaagcatggttgaatgactcgcccaaggccacatggctaataaggaacagagccggtctgcgagactctaagtgactgaagaacttcttatgtgtggtttctgttcttggcagggagtcttagtgggctccagggcagggggaaaagagagagctacatgagaatgggcatgcccgtcttccaacagccatctccactgtccaaccgttgctaggcaacatgaagcccatgactgcagctcttcaggaggccgaggcagggagatcacaagttcaagcctacctggggctgcaaagtgagttcaaagctggggcagcttagtgagaccttgtntcagagtggtaaagtaaaaggatgggtaaggatgtaanagcncagtggaaagggactcggccaanggggtgaagnactaagggcacgagnattcaanccccaagnaactgcanaagaagnggggng

SEQ ID NO: 124 - DNA, 987 bp, Rattus norvegicus
gcaagcgcgaaattaaccctcactaaagggaacaaaagctggagctccaccgcggtggcggccgctctagaactagtggatcccccgggctgcaggaattcggcacgaggcgtgcggggcgggggcgcccgacggcgtccgagggcgcggcggacgaggcctgagggaggggacgcgatgctggagaccctgcgcgagcggctgctgagcgtgcagcaggatttcacctccgggctaaagacgttaagcgacaagtcaaaagaagcaaaagtaaaaagcagacccaggactgctccctacttaccaaagtactctgctgggctggacttacttagcaggtatgaggatacgtgggctgcacttcacagaagagccaaggaatgtgcagacgctggcgagctggtggacagcgaggtggtcatgctgtctgcccactggggagaagaagaggaccagcctggccgagctgcaggagcagctgcagcagctgccagctctcctccaggacgtggagtccttgatggcaagcctggctcatttagagacgagttttgaagaggtagaaaaccacctgttgcatctggaggacttgtgtgggcagtgtgagttagaaagacataaacaggcccatgcccgacacctggaggattacaagaaaagtaagaggaaggagcntgaagccttcaaagctgaactcgatacagaacacgcacagaagatcctggaaatggagcacacccagcagctgaagctgaaaggagcggcagaagtttctttcgaggangctttccagcaggacatggagcaatacctgtccacangtcacctgcagattgcaganaaggcgagagccccattggggcagcatgtccttccatgggaagtgaatgttggacgtncttggagcagaatggacctngattggaccctctttngacccaagaanggggctcggatggccttcctttaaacnnctggggggngang

SEQ ID NO: 125 - DNA, 998 bp, Rattus norvegicus
caagcgcngaaattaaccctcacgtaaagggaacaaaagctggagctccaccgcggtggcggccgctctagaactagtggatcccccgggctgcaggtggaggctgagattgtacagcaacaggcacccccttcctatggacagcttattgcccagggtgctatcccacctgtagaagacttccccacggagaaccccaatgacaactctgtgctggggaacctacgttctctgcttcagatcttgcgccaggatatgactccaggaggtacttccgggggccggcgtcgccagcgtggccgctcagttcgtcggctggttcgcaggctccgtcgttggggcctgcttcctcgaacaaatactccggctcgggctcctgagaccagatcccaggccacaccttctgttccctctgaggccctggatgacagcacaggtcaagcctgtgagggtggggcagtaggagggcaggatggggagcaggctcctccactacccatcaagtcccccataccaaccccaagcacacttccagcccttgctactgcctctgaacctccagggccactaccctcagtgcctgtagaatcatcactgttgtctggagttgtccaggttcctaaggaggccgcctcctacccagcctgtggcccccaggncccaattggaccccaatggaactcacacagcagtcctgccttctagaggatgaggatgatgtatgttgatgccattggctgagccaagaagtctgggtggtttgaggcagaggatgaancaatggctttgcctgagagaactctagacacaatgtaatgaatctggtggnaagtgggttcagaaaagttaagtgtcccctcgaagggggggggcccgggtaaccccaaattgggccctaanaagtggagtncggaattaaaaattcaantgggccngtnnggttttaaaaaaggnnggtgaantnggggaaaaacccttgggggttaaccaaactttta

SEQ ID NO: 126 - DNA, 978 bp, Rattus norvegicus
aagcgcgaaatnaaccctcactaaagggaacaaaagctggagctccaccgcggtggcggccgctctagaacnagtggatcccccgggctgcaggctaaactttattgacaaacttgctgcagacattttgacataggttaagctacctatttaggcagacttacacatgtagcatttaatcttccaaaaacaaaatgggaggacggtacaaatccattaggactttaacttatgtacaaagtggactttgattctcttctcattcagctgcagtgtccctttttatgtcatgctagtgttgagacatacttaactaccgtggcaacagtgcgaaactgacaatggtcaacttaatgaacagacgtcacttttcggnccccagtgtccaagtgnagtttttcatggagtgcagaatctcagatggacaaaatacncttggacattttaaatactgaaaatttggattatncagtactattattgaaaagactgtggctaaaaagaactgtcagacnccattaggcggccagcttnccnccccagcaacctattcaacccccccccccactaagtatctctcaacacngtatgtctggggctagatttcaaaacccacagaatgaaaaaggcattttacaaacctaaattttgttgttgttttaagncaatttaacgntnaaaaatngcatccaacnatttaantcatgagatctttcntatnaaanattnaaaccntaagnattcaacccggccangnggcttttaaaaggggaaatgntttttagnagacaatccngnggcncccctttttacnaagggggggcaccnaaaggggccggtnaanaantggtgaattnttacaggcntaaagccagncccnggaaattngaaanggaggccccagtttnggggaatccnggcaaccnanggncntnnancggggggggcccacaaaancnaaaggccnaaagnnaaaan

SEQ ID NO: 127 - DNA, 936 bp, Rattus norvegicus
agcgcgaaattaaccctcactaaagggaacaaaagctggagctccaccgcggtggcggccgctctagaacnagtggatcccccgggctgcagggtgaacctcagttctccatcaaacatgcttcctgtccggccacaaactaaccctctgatggggggacccatgcctatgaacatgcccggtgtaatgacgggcaccatgggaatggcccctctggggaacaccgcaggatgagccagggcatagtgggcatgaacatgaacatggggatgtcggcctcggggatgggcttgacaggcaccatgggaatggggatgcccagcatggccatgccgttctggaactgtgcagcccaagcaagatgacctttgcaaactttggccaactttagtaaataaaaggttgtaacggagcgagtggaagaagcctctgtagctgcaataggtgatgttgggctggaagatgctaagcagttcccttttctttcatcagttaattaaataaccacataaagaaccaaaaaggctgctgtttcagaagcgatgcaagagcacttcagacgaggcagtcaggatcggtttccccagtgaagatacatacgctcctaaatggggcgagggggcacgagagcctctctgtcagagagcatgtgtcccagcgtagtctgtgggaggactggcatggatgggggctgagtaagtgtgcttcactctctaactttatactttctctccctgaggaanttgattttctgtccctcagncgccttgtcatgantgggnctgttcctttantaccaatctccaagtccaaggtaatgaaancattaaaagttngggngnatcagntttttttatnaaaaatataaaanggnggggccaaaaaaaaaagggatanccaanggggaattatgggcngagtccaaagggaaccnng

SEQ ID NO: 128 - DNA, 931 bp, Rattus norvegicus
gcaagcgcgaaattaaccctcactaaagggaacaaaagctggagctccaccgcggtggcggccgctctagaactagtggatcccccgggctgcaggaattcggcacgagattaggagtaccagcctgctctaacggtttcagggaagattggctgtgggtttccgcagagtgtgggggagttcctgcttatccaactggctcgccatggcttccctgtgggcaagggcttggcaagagccctggacaaaaaacgggacatcattgagaagacacctgctttgtgcgaggtgttctgccggcaagggagggggggcccggtacccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcacctgnaatggcgaatggcaaattgtaagcgttaatattttgttaaaattcgcgttaaatttttgttaaatcagctcattttttaaccaataggccgaaatcggcaaaatcccttataaatcaaaagaatagaccggagatagggttgagtgttgttccagtttggaacaagagtccactattaaagaacgtggactccaacgtcaaagggcgaaaaaccgtctatcaggggcgatggcccactacgtgaaccatcaccctaatcaagttttttggggtcgaggtgccgtaaagcactaaatcggaaccctaaanggagccccccggatttagagcttgangggggaaagccgggcgaacgtgggcgagaaaagggaagggaagaaaagcggaaaggagcggggcgctaagggccctgggcaaatggtaaccgggt

SEQ ID NO: 129 - DNA, 936 bp, Rattus norvegicus
gcaagcgnggaaattaaccctcactaaagggaacaaaagctggagctccaccgcggtggcggccgctctagaactagtggatcccccgggctgcagggattgacggttctgagggtgaagggaagctgggtctgtaacctagtatctctgacaagacagtgtttacatgagctaatcctcttcaagactcagagctgagacaaagtcatttttctttcagtttttgttgtcacacctttttttattttattgtattaaaacctagccataatgaagatagaatttctgtttacattttgggcatgatgtggctgcatgcagaggcttcatgcttttgaaccctgtatttgattgtgctgcatgggaggcttttattcttggacagcttagtacagaagcaggagaaggtttgagttcttggggatgcaaaggacggatggcctatattctaaagacagtgtcatcgcccttcctgtgtgctgacccagggctgtgtgtgtgctaggcgagtgctgttgaaaggtagacacgctggtggagagaaacaagctgctctcatcaccacacacttctgcagagcctttgtgctgcagctccccgtgaggctgtcctccagttctctccagccagaattgctgtggcaacaatatttttataaagcagtgggcttcatcttaggaccagtcaattaataagcgtggccgtagctgagaagagccactttccaagagcgcaccagacacagtgagtggtgatcagcccccttctggcctgcctgtatgattgagaatcccaaaaactctggtaaatccataagtgggggaacagaaggcaccggatccttccaataagccagagaagggaantnggggctttaaggaccatttggtgccaaaaagggttttttgggggggnttgggggggaggtcccgtttt

SEQ ID NO: 130 - DNA, 955 bp, Rattus norvegicus
caagcgcgaaattaaccctcactaaagggaacaaaagctggagctccaccgcggtggcggccgctctagaactagtggatcccccgggctgcaggccagcctctgtctctgaggagagctgttagagacttgactgaggaaagccagcacggagtcccagaatttaaaccgtgactatactttaaacatttactagcactccctagccagccctgtcagaggataacagcagcactagctgacattactgaacagtgctggtcgagactgctttgtgtaagtgtcttgaccaatcttcaagagaactctgtgaggttactatgattatccaaatgctactgaggaaaacagaagattagagatactggacttattcagattctggcaactattaaatgggcaggacgtaatcgttactgtggtcagaaaatcccccttttagatgagattccagcccctctcctaatgcctcaggttcacaaggaaggcaagagagggcagaacccagagggatgtggtcatgagtgtgggtagggaaaagtgcaggaagctgagaataggattgctactggagtattgatgggattgcagagcggtcccaggtaatgcccctaagtatggcaccattcccatgaaaaaacactcagggcaagcagggtgaactctcaactccaaatatttacgtgctaaaattcctagaaagtgacactggactctacctggtcgtgaagttccctatttgggtctctaacaattatcttctgttcacacangggatccctgtatctcaagtctcccatggagattccaggcctttcaanggggctggggggagttgaaagggggagccactggggccttgaaagggggggccactngggcccttggtttngtcccngngggctaaaagggcacggggggtaaatccaaggttccccctgggnnaacaagggaa

SEQ ID NO: 131 - DNA, 929 bp, Rattus norvegicus
aagcgcgaaattaaccctcactaaagggaacaaaagctggagctccaccgcggtggcggccgctctagaactagtggatcccccgggctgcagggctgaggcagccatcttgctcttgccgcgtgctggtgttagaggaccctccccgctgcagatttaccaacagcatgaatcaagaaaagttagccaagcttcaagctcaggtccggatagggggcaaggggacagctcgcaggaagaagaaggtggtgcataggacagctactgctgatgacaaaaagcttcagagttcactaaagaaactggctgtgaacaatatagctggtattgaagaggtgaacatgattaaagacgatggaacagttattcatttcaacaatcccaaagtccaggcttccctctccgctaacacctttggcaattactggtcatgcagaagccaaaccaatcacagaaatgcttcctggaatactaagtcagcttggtgctgacagcttaacgagccttagaaagttagctgaacagttcccacgacaagtattggatagtaaagcacccaaaccagaagacattgatgaagaggatgatgacgttccagatcttgtagaaaattttgatgaagcatcaaaaaatgaagctaactaaaatcttctgggttttggaagctggcatggactagatttaacaatcagctctgttgttccaaagttttacagacatggagaacatcacctgttattagttccgtaatataaatgtnnngtatattaatgatgctgttttatcagcatttcctggtcattgggattttgcattttgcacttcttcccagggatcggattcctttgggncaaaatatggaggaattgggtaccagggtgaaggggtggttttggnttttttggggggggnccttttgggnggtggaa

SEQ ID NO: 132 - DNA, 730 bp, Rattus norvegicus
gaggagctgtgccggcagatccagcaggaggaggacgaaaagcagcggctacagaatgaggtgaggcagctcacggagaaattggcccgcgtcaacgagaacctggcgcgcaagatcgcctctcgcaacgagtttgaccggaccattgcggagacagaggccgcctacctcaagatcctggagagctctcagactctgcttagtgtaatcaaaagagaagctgggaacctgactaaagccacggcttcagaccagaagagtagtggaggcaaagacagctgacaagccctgtctcagccgtggcctatggctgctccccaacatgtctgtcctaaagcatctttgttctccatggcctcagatgtcttttatctctggtgccctgagtgtcaatttctgacctccacctgccttgagtgacaagacaagccccaggacagtccaatggaagatgtgttcccagctccgcactcacatgtgttattggaaccttgagctcctggtcagctccatgaggagcctctttctgaagctcctgtatctccttgggctgccaggaatgtctcggtcctagtccaggtactgtgggagcccctcactttgtctcctcagccactagggccccaggccaggcatattgaagaaagaatcttggctccagaaccaaaaccttagggcccatccccatgacctctggtgtttcaataaaggtgtttgcaaaaaaaaaaaa

SEQ ID NO: 133 - DNA, 709 bp, Rattus norvegicus
ttttttttttttttttttaggactcatgcctctctttattcagatttccccacccacttacagcaaattgtgaacccagcctcaggtctggggtcaggggccctgaacctccctaggattccccagctctgtccagcaacttttctttccagttcacagacagagagatggagacccagtgggcacagccagtctggcaatctttaataagaagagaggccttggccagggctctgtatgtcctcgctgggctgtggttgtctctggctggggttcagggagctgttgatctggtgtgtgagggtttccagtcttggggtctggtttgggatcccagatcctcctcttagcacttgtggcatgtttgagggccctgcaggcaggggtggtgcagagggtaccacttcctctcccatcagcatctctgccaggactcgtgtctgtgtccagtcactatggttcctctcttgtgggggcttggcagagcctgggcttgttggaggtgcccagggtgggggccgcacagatggagatatgtggcctggggcctcaggctccagcacctgctgtggtgcatggcattctctccatgggcaggcccagtgggtcaaggacactcggcacagaggacagaggtgatagcagtagcagaggggtggcagtacatggcagaggcagctggagtcatcctgcagggcacacggtggca

SEQ ID NO: 134 - DNA, 376 bp, Rattus norvegicus
gattctatggcttcagtgtgaagaactgtggagcccagcctgccctgcacacctggaccctgtccctgcactgcctgtgttcccttccacagccaaccttgctggtccagcctgggggtagggggtggggacatctgcatcctgtcactccttgctgccctgagcttcagcccctcactccacactgagaataagaatctgagtgtgaacttgattgttcacatccttgacacaagtgtggatggctttttaaattactggatggaatacttagaggtttttgttttggggttttgctttgttttgttttgtttttgaaaaaaataaatggcagatgaagaagcttccaaaaaaaaaaaaaaaaaaaaaaaaaaaa

SEQ ID NO: 135 - DNA, 723 bp, Rattus norvegicus
ttttttttttttttttttttagctttcaaattgctttaatacttgcacttacaagcaacaggtaaacaacgaacagttattactgggcagtgtcacaggggtgggggctggagaggggagtagaggatcaagcaagcttcacacaaaggcgtttatgtgaaggacaggaaacacgggcatgctaaattctgcagagaatacaacacatacacactacaggggctggagacgtggatgacattaagaaacatgtctgaaatatgaagcttggtatacagtttatatgtggagtgtccccggtgatggccattcttggttgtcaacttgactccatcttgaatgaactaaaatctaatgacagagggcacacctgggagggagttttgcttaatttgaagtcagtagatcttctttaatcctgatcttgaggtgggaagacacacctctagtctgggccacgccttctgctagaagtctacttgaggacaggacgaagggagcttttgctctttgcctgcttggcctcaccttgttggcaagtccattccttcactggcgttagagcctacgtcattgggattccagagtctactgaagaccagccgagacacccagcctcatggcccgaactgctggatttttggactttctgctcactgttggattagctggacagaagcctgtaagtcattcttcttctgggaattccttctgtaagtgctg

SEQ ID NO: 136 - DNA, 594 bp, Rattus norvegicus
ctccccttcatgaacttcctgcctatcacttattgcctttcctaattaatgttgagaattttacaagccgtattgattgacccacctgttcatccatggcagaatgacgcgtctttgtctactgtcttctttgtttactatatgcccaaggttattccagacatgggaaatacagacacacatgtttctgttcctctctgtgatgggcacacaatccaaggagcacatcctgcacttttcaaaactgaacataactagatcagctggtgactttgtcacttggctgaaatccccagaataagccaattccatcctaacctttgcatatccagtacgactacccaacattgaagaacaatgattgttcttagagtctaatgaattttcaagtatttcttacccgaaaaataccaagaactatgcaaaaatggaaaacaaaggtttagttacttacaagctttaaacttatggatgtaaggaatgtgtacctgatttaagatggtgatgagcccacagtaggcttatttacttgaaaacattttagtcatcatgtatttccggttgctaaatgtgcttgtgtttataaaattcaaa

SEQ ID NO: 137 - DNA, 433 bp, Rattus norvegicus
gagtgtggggctggagagatggctcagtggttaagagcacccgactgctcttctagaggtcatgaattcaattcccagcaaccgcatggtggctcacaaccatctataaagagatccgatgccctcttctggtgtatctgaggaaagctacagtgtacttatatataataagtatagaaatctttaaaaaaaaaaaaaaagaatctgagtgtgaacttgactgttcacatccttgacacaagtgtggatggctttttaaattactggattgagaatacttagaggttgttttttggttttgttttgttttcttttgttttaaataaatggcaggtggagaagctcccaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 138 - DNA, 619 bp, Rattus norvegicus
tgaatttagtgctgaacatgaagagtaaactatttaccaaaaagaagttcctggagtttggagaagtaacgaatgtatccatctgtacatgatttacatgttgtggatgctttgtaaacatttccccatgttttaattgtgtttcagcaggttgtaattgcctctgtgtgtagctgaacatgagtcattatctggtcctgtatgaaatggaatgtatggtattttctgtatcattttcctgaggctgtgtttggggagccacacattcgaatacagttttcctgatcacttgatttcttgtgcacctgatttttgctctcaaaggaattactgccacaatatattttatttattctttagattttagccttgtcagttgaagtgcttcacatgatggtgttaaaaactacttgtccctttactgggggtttgggggttgttaaaagatggggaggaagaatgcaaatgggtcattgttaacctgtccccactgatcccacctgtactcatagtcccttccaggatggtattctgatgtttcctacaatacggtgaccataggcaacttgttacctgaataaaggatcgattttaaacagccaaaaaaaaaaaaaaaaaa

SEQ ID NO: 139 - DNA, 1018 bp, Rattus norvegicus
actngaaattaaccctcactaaagggaacaaaagctggagctccaccgcggtggcggccgctctagaactagtggatcccccgggctgcaggattgaccaggcccataggcagaatgtctcctctctttttctgccagtgattgagtctgtgaatccttgcttaattctggttgttcgcagagaaaatattgtaggagatgcaatggaagtcctcaggaaaaccaagaatatagattataaaaagccactcaaagttatatttgttggagaagatgctgttgatgctggaggtgtacgcaaagaatttttcttgctcatcatgagggaattattggatcctaaatatggcatgtttcgatattatgaagattccagggctatttggttttcagataagacatttgaagacagtgatttgttccacttaattggtgttatctgtggattagcaatttataattttactatgtggacctccacttccctttggctttatataagaagctactgaaaaggaagccatccctggatgatctgaaagagctgatgccagatgtaggggagaagcatgcaacaattgctggactacccggaggacgatatagagggaaacatttgtctaaactttacgatcacagttgaaaatttggnggcaacaggaagtgaaagagcggntctgaaagggtggcagacancgctggttaacaaacagnaatcgggcagggagtntgtgaagccnaaggngggntaccanatncganaaaatncagngggnnnctttaaaaaaggggctttcccaaggccgggntttccataagggtncngngggngggaaaaaagtncttccgggnnncntccnagnccccaaanggaaatttannaaggggnanggggnnaatnggggaannaccgaanntaangnnntgggggaaggnaacntgggnnngangaaaganncngggnngnncnnnangnaaagggnannngggggggggaaaaacccccnnggngnnaaag

SEQ ID NO: 140 - DNA, 371 bp, Rattus norvegicus
tcctagcacaggggctccaatgccctgggcagcagaggattatggtctaagccgtttgagtaatcatcggcttcttcccagcacattggtgaggaaacaggccacgacttgtcactcagcactaacccccagttgttgaacagccttctccagccctgctttaggatgacaaatgaataacacctaggcatagaaaccagtctctctggtttgtttgtattatgttcttcaacattaaagatttaaacaacaaaggatatactacagtcttgaatctaaagtaatttgctaactattttgattcttcagagaactactaataaaaatctaaaaggtaaaaaaaaaaaaaaaaaaaaaaaaaaaaaaaaaaa

SEQ ID NO: 141 - DNA, 1024 bp, Rattus norvegicus
aagcgngaaattaaccctcactaaagggaacaaaagcaggagctccaccgcggtggcggccgctctagaactagtggatcccccgggctgcaggctttttcgcacatcccgacctgttcgtgagcattagtgatcagaaggaccccagggatcggatggttcaggttgtgaaatggtacctctcggccttccatgcaggaaggagaggatcagtggccaaaaagccgtacaatcctattttgggtgagatctttcagtgccactggacgttgccgaatgatactgaagagaatgcagagctcgtttcagaagggccggttccctgggtgtctaagaacagtgtaacatttgtggctgagcaagtttcccatcatccgcccatttcagccttttatgctgagtgttttaacaagaagatacaattcaatgctcaattgaaggggaatggaatggcatcatgtatgcaaaatatgcaaccggggaaaaaactgtctttgtagacaccaagaagttgcctataatcaagaaaaaagtgaggaagttggaagatcagaacgagtatgagtcccgaagcctttggaagatgtcactttcaatttaaaaatcagagacattgatgcagcaacgggaagcaaagcacagacttgaaggagagacaaagagcagaagcgcgagaacgggaaaggagaagggaaattccagtggggagacgagggctctttccacgaaganggaagaatgccggggtttacgnatggaancctttantggaaagcggncttggggtacctggggaagccattaaagccngaaanccggggttccaccgggggtgnacccagggggcantnnggcgnaacnnaaggnnaacaaatcngnttcttccagggggnaancttgnccacttncccttncnttaaaaggggggggggtncccaaancnccnggggganancgggganttngannncnggnnggaccnaattttaaaagggggaaaggnggnttnccccntttttaaang

SEQ ID NO: 142 - DNA, 790 bp, Rattus norvegicus
gcggctgtgaccgagagcacagagaatgaaccagccttgcaggagaaaacactacaaattagctgacagctggtagagaagatgctctatttctgtgctcagtgcctgtgggatctgtgatggaaatgatggccggctgtggtgaaattgatcactcactaaatatgcttcctaccaataagaaggcgagtgagacctgttctaacactgcaccttctctaacagttcccgagtgtgccatttgtctacaaacatgtgttcatccagtcagtctgccctgtaagcatgttttctgttatctgtgtgtaaagggcgcttcatggctcgggaagcgatgtgctctttgtcggcaagagattcctgaggattttcttgacaagccaaccttgttgtcaccagaagaacttaaggctgcaagcagaggaaatggtgaatatgtgtggtattatgaaggaagaaatggatggtggcagtatgatgagcgcaccagtcgggagctagaagatgctttttccaaaggtaaaaagaacacggaaatgttaattgctggatttctgtacgttgctgatcttgaaaacatggttcaatataggagaaatgaacatggacgtcgcagaaagattaaaagagatataatagatataccaaagaagggagtggctggacttcggctggactgtgacagcaacactgtaaatctagccagagagagttctgccgatggtgcggacagtgggtcagcacacactggagcttctgtgcagcttccagtgccatcttctacaggcct

SEQ ID NO: 143 - DNA, 19 bp, Artificial Sequence
  forward primer for amplification of TRDH-344 DNA
agggtagaagtggagtctg

SEQ ID NO: 144 - DNA, 20 bp, Artificial Sequence
  reverse primer for amplification of TRDH-344 DNA
caaaggcacattgtgaggga

SEQ ID NO: 145 - DNA, 20 bp, Artificial Sequence
  forward primer for amplification of TRDH-271 DNA
tgaagtaggaatgctggtct

SEQ ID NO: 146 - DNA, 20 bp, Artificial Sequence
  reverse primer for amplification of TRDH-271 DNA
acgatgtactccaccagctt

SEQ ID NO: 147 - DNA, 19 bp, Artificial Sequence
  forward primer for amplification of TRDH-284 DNA
agaacatgccactggtcgt

SEQ ID NO: 148 - DNA, 19 bp, Artificial Sequence
  reverse primer for amplification of TRDH-284 DNA
acagtgcagaccgatctca

SEQ ID NO: 149 - DNA, 21 bp, Artificial Sequence
  forward primer for amplification of TRDH-363 DNA
gggtatgggatgacctgaaca

SEQ ID NO: 150 - DNA, 21 bp, Artificial Sequence
  reverse primer for amplification of TRDH-363 DNA
agcacaggtactgcagggatg

SEQ ID NO: 151 - DNA, 18 bp, Artificial Sequence
  forward primer for amplification of TRDH-292 DNA
ggcgtccgacgatgccaa

SEQ ID NO: 152 - DNA, 20 bp, Artificial Sequence
  reverse primer for amplification of TRDH-292 DNA
gccctacagagtcttacaca

SEQ ID NO: 153 - DNA, 20 bp, Artificial Sequence
  forward primer for amplification of TRDH-122 DNA
gagcaaggtccttccatagt

SEQ ID NO: 154 - DNA, 20 bp, Artificial Sequence
  reverse primer for amplification of TRDH-122 DNA
atgtcagcaggagtgggtta

SEQ ID NO: 155 - DNA, 20 bp, Artificial Sequence
  reverse primer for amplification of TRDH-110 DNA
agattgtcccaacagagagg

SEQ ID NO: 156 - DNA, 20 bp, Artificial Sequence
  reverse primer for amplification of TRDH-110 DNA
gacaggaaatggtgatgcta

SEQ ID NO: 157 - DNA, 883 bp, Rattus norvegicus
gccggcagggggcactgcgcgccgggcatggagtgcgtgaagagccgcaagaggcggaagggtaaagccggggcagcagccggcggtcccgcgaccctcgccgtgtgcgtgtgcaagagccgctacccggtgtgcggcagcgacggcgtcacctaccccagcggctgccagctgcgcgccgccagcctgcgcgctgagagccgcggagagaaggccatcacccaggtcagcaaaggcacctgcgagcaaggtccttccatagtgacgccccccaaggacatctggaacatcactggcgccaaggtgtacttgagctgcgaagtcatcggaatcccaacccccgtcctcatctggaacaaggtaaaaagggatcactctggagttcaaaggacagaactcttgcctggtgaccgggaaaacctggccattcagacccggggtggtccagaaaagcatgaagtaactggctgggtgctggtatctcctctaagtaaggaagacactggagaatacgagtgccacgcgtccaattcccaaggacaggcttcagcgtcggccaaaattacagtggttgatgccatacacgaaataccagtgaaaaaaggtgaaggtgctcagctataaacctgcgaatacattagcctctgtagctgacgcgctctcagacagctgacagctgtaaccccactcctgcctgacatattcctttgaacctaacacactaacactttattacagccagctgattttacagagaaatcaaagataacacataagactatctacaaaagtttattgtttatttacagaaaaagcatgcagagctttaaacaaaacaaataaaattcttattacaacaggaaaaaaaaaaaaaaaaaaaaaa

SEQ ID NO: 158 - protein, 207 aa, Rattus norvegicus
AGRGHCAPGMECVKSRKRRKGKAGAAAGGPATLAVCVCKSRYPVCGSDGVTYPSGCQLRAASLRAESRGEKAITQVSKGTCEQGPSIVTPPKDIWNITGAKVYLSCEVIGIPTPVLIWNKVKRDHSGVQRTELLPGDRENLAIQTRGGPEKHEVTGWVLVSPLSKEDTGEYECHASNSQGQASASAKITVVDAIHEIPVKKGEGAQL

SEQ ID NO: 159 - DNA, 1120 bp, Homo sapiens
  CDS (211)..(1086)
ctgggccagctggtggtgcccagcaaagccaaggcagagaaacccccactgtcggcctcctcaccccagcagcgccccccagagcctgagaccggtgagagtgcgggcacatcccgggctgccacgcccctgccctctctgagggtggaagcggaggctgggggctcaggggccaggacccctccactgtcccggaggaaagctgtagacatgcggctgcggatggagttgggtgctccagaagagatggggcaggtgcccccacttgactctcgccccagctccccagccctctacttcacccacgatgccagcctggttcacaaatctccagaccccttcggagcagtagcagctcagaagttcagcctggcccactccatgttggccatcagtggtcacctagacagcgacgatgatagtggctccggaagcctggttggcattgacaacaaaatcgagcaagccatggacttggtgaagtcccacctcatgtttgcggtccgggaggaggtggaggtgctgaaggagcagatccgggaactggcggagcggaacgctgcgctggagcaggagaatgggctgctgcgcgccctggccagcccggagcagctggctcagctggccctcctcgggggtcccacggcttgggccccctgcgcccaatgggccctccgtctgagcctcccttcccttacaatgtgcctttggggctgcccggccttgcgtcagccgcctgccccctcttcctatgcagctttaatgtccccgtgtccccggggtgggagttcaaggctcagtaatggcctggtcccccggcccctgccccatctcctcatcatccccagccttgatggaggagggagggcttcaggacggggcgtcagagggagccccctctgggagggaaccaacccccaccctccctcctgggaccccccagcagtagacggcttgggggagtcggaggctccccggcagacaccccacccccatcttgttcccttgaggtgcctcctctcctctgcccaggggagggagtgtggacagtatctggaagttctgggattcaggttgttattaaaataataataataattaaaaactctgaagaaacttg

SEQ ID NO: 160 - protein, 292 aa, Homo sapiens
MRLRMELGAPEEMGQVPPLDSRPSSPALYFTHDASLVHKSPDPFGAVAAQKFSLAHSMLAISGHLDSDDDSGSGSLVGIDNKIEQAMDLVKSHLMFAVREEVEVLKEQIRELAERNAALEQENGLLRALASPEQLAQLALLGGPTAWAPCAQWALRLSLPSLTMCLWGCPALRQPPAPSSYAALMSPCPRGGSSRLSNGLVPRPLPHLLIIPSLDGGGRASGRGVRGSPLWEGTNPHPPSWDPPAVDGLGESEAPRQTPHPHLVPLRCLLSSAQGRECGQYLEVLGFRLLLK

SEQ ID NO: 161 - DNA, 66 bp, Artificial Sequence
  primer with T7 promoter and poly thymidylate sequence
aaacgacggccagtgaattgtaatacgactcactatagggcgtttttttttttttttttttttttt

SEQ ID NO: 162 - DNA, 19 bp, Artificial Sequence
  forward primer for amplification of G3PDH DNA
atcaccatcttccaggagc

SEQ ID NO: 163 - DNA, 21 bp, Artificial Sequence
  reverse primer for amplification of G3PDH DNA
caccttcttgatgtcatcata